(19) United States
(12) Patent Application Publication
(10) Pub. No.: US 2011/0311484 A1
(43) Pub. Date: Dec. 22, 2011

(54) STRAINS OF XENOTROPIC MURINE LEUKEMIA-RELATED VIRUS AND METHODS FOR DETECTION THEREOF

(75) Inventors: Judy A. Mikovits, Reno, NV (US); Vincent C. Lombardi, Reno, NV (US)

(73) Assignee: Whittemore Peterson Institute for Neuro-Immune Disease, Sparks, NV (US)

(21) Appl. No.: 13/081,289

(22) Filed: Apr. 6, 2011

Related U.S. Application Data
(60) Provisional application No. 61/321,147, filed on Apr. 6, 2010.
(60) Provisional application No. 61/358,734, filed on Jun. 25, 2010.

(51) Int. Cl. A61K 38/21; A61P 31/14; C12Q 1/70; C07K 19/00; C07H 21/04; C12N 9/12

(52) U.S. Cl. 424/85.6; 536/23.72; 435/194; 435/5; 536/23.2; 530/350

(57) ABSTRACT

Provided are novel strains of Xenotropic Murine Leukemia Virus-Related Virus (XMRV), or polynucleotides or polypeptides thereof. Identified herein are nucleic acid changes or amino acid changes identified in XMRV strains isolated from subjects. Also provided are methods of detecting such XMRV strains based at least in part on the identified nucleic acid changes or amino acid changes.

### CROSS-REFERENCE TO RELATED APPLICATIONS

This application claims the benefit of, and priority to, U.S. Provisional Application Ser. No. 61/321,147, filed Apr. 6, 2010; and U.S. Provisional Application Ser. No. 61/358,734, filed Jun. 25, 2010; each of which is incorporated herein in its entirety.

### STATEMENT REGARDING FEDERALLY SPONSORED RESEARCH OR DEVELOPMENT

This invention was made with Government support under Grant RO1A1078234 awarded by the National Institutes of Health. The Government has certain rights in the invention.

### MATERIAL INCORPORATED-BY-REFERENCE

The Sequence Listing, which is a part of the present disclosure, includes a computer readable form comprising nucleotide or amino acid sequences of the present invention. The subject matter of the Sequence Listing is incorporated herein by reference in its entirety.

### FIELD OF THE INVENTION

The present invention generally relates to the isolation of variants of xenotropic murine leukemia-related virus (XMRV).

### BACKGROUND

Multiple Sclerosis (MS), Amyotrophic Lateral Sclerosis (ALS), Niemann- Pick Type C Disease, fibromyalgia, autism, chronic lyme disease, and Chronic Fatigue Syndrome (CFS) are examples of neurological diseases believed to involve malfunctions in the immune system.

Patient selection poses a challenge to any study of neuroimmune diseases, because of the variability of patient symptoms. For example, chronic fatigue syndrome (CFS) is a debilitating disease that affects more than one million people in the US alone. CFS is a disease characterized by severe and debilitating fatigue, sleep abnormalities, impaired memory and concentration, and musculoskeletal pain. In the Western world, the population prevalence is estimated to be of the order of 0.5%-2% (Papanicolaou et al. 2004. Neuroimmunomodulation 11(2):65-74; White. 2007. Popul Health Metr 5(1):6). CFS subjects are known to have a shortened life-span and are at risk for developing lymphoma. Currently, there is no diagnostic test and no treatment, except for the specific treatment of microbial infections in those cases in which microbial agents can be identified (Devanur and Kerr. 2006. J Clin Virol 37(3):139-150). Although the precise pathogenesis of CFS is unknown, a range of factors have been shown to contribute (Komaroff and Buchwald. 1998. Annu Rev Med 49:1-13; Devanur and Kerr. 2006. supra). Furthermore, a single patient with a bona fide CFS diagnosis can present with variable symptoms over the duration of the illness.

Several retroviruses such as the MuLVs, primate retroviruses, HIV, HTLV-1 and XMRV are associated with neurological diseases (C. Power, Trends in Neurosci. 24, 162, 2001; Miller and Meucii 1999 TINS 22(10), 471-479; Power et al. 1994 Journal of Virology 68(7) 4463-4649). Investigation of the molecular mechanism of retroviral induced neurodegeneration in rodent models revealed vascular and inflammatory changes mediated by cytokines and chemokines and these changes were observed prior to any neurological pathology (X. Li, C., Hanson. J. Cmarik, S. Ruscetti J. Virol. 83, 4912, March, 2009, K.E. Peterson., B Chesebro. Curr. Opin. Microbiol. Immunol. 303, 67 2006). Neurological maladies and upregulation of inflammatory cytokines and chemokines are some of the most commonly reported observations associated with CFS. Retroviral involvement has long been suspected not only for CFS but also for other neurological diseases such as Multiple Sclerosis (MS) and Amyotropic Lateral Sclerosis (ALS) (E. DeFreitas et al., Proc Natl Acad Sci USA 88, 2922 (Apr. 1, 1991); A. Rolland et al., J Neuroimmunol 160, 195 (March 2005); A. J. Steele et al., Neurology 64, 454 (Feb. 8, 2005)).

Retroviruses have also been associated with various cancers. For example, the gammaretrovirus XMRV has recently been implicated in prostate cancers (Dong, B., et al., Proc. Nat'l. Acad. Sci. USA 104, 1865-1660, 2007; PCT patent application PCT/US2006/013167, published as PCT publication number WO2006110589 of Silverman et al.), mantle-cell lymphoma, and chronic lymphocytic leukemia lymphoma. HIV-positive patients are known to have increased incidence of Kaposi's sarcoma and lymphomas. Subjects with HTLV-1 exhibit increased rates of leukemia and lymphoma, including T-cell leukemia/lymphoma and B-cell chronic lymphocytic leukemia.

Phylogenic analysis of published XMRV sequences indicate that this virus is closely related to but distinct from endogenous retroviruses found in the mouse genome. Endogenous murine leukemia viruses (MLVs) can be classified as polytropic, modified polytropic, and xenotropic MLVs (Stoye and Coffin 1987 J Virol 61(9), 2659-2669). Among these, XMRV genomic sequences are most closely related to MLVs (i.e., X-MLVs), although the nucleotide sequence of XMRV differs by at least 5% from any X-MLV found to date.

The XMRV genome encodes, in 5′-to-3′ order, the 3′ long terminal repeat (LTR); a short, apparently non-coding sequence comprising a splice site acceptor (“SA”); the Gag gene; the Pro-Pol gene, comprising a splice donor site (“SD”), the extreme 3′-end of which overlaps with the 5′-end of the Env gene; the Env gene; another short non-coding sequence; the 3′-end LTR; and a poly-A tail (see e.g., FIG. 1).

XMRV sequences published to date show little sequence diversity. The full-length sequences of XMRV genomes isolated from infected individuals available in GenBank have 99.4% nucleotide identity (see Knouf et al. 2009 J Virol 84(14), 7353-7356; Lombardi et al. 2009 Science 326(5952), 585-589; Urisman et al. 2006 PLoS Pathog 2(3), e25).

### SUMMARY OF THE INVENTION

Among the various aspects of the present invention is the provision of a novel XMRV polypeptide and polynucleotide sequences as well as method for detecting such.

One aspect provides an isolated XMRV polynucleotide. In some embodiments, the XMRV polynucleotide has a nucleic acid sequence according to SEQ ID NO: 1 and one or more nucleotide sequence changes selected from the group consisting of C80T, G90A, A96G, A97G, G111A, A137-157 deletion, T173C, G180A, G183A, C197T, C247T, C257T, C308T, C308G, C319T, C320T, T326C, A329G, C715T, T791G, A804G, T816Del, A856G, A665Del, T691G, G790A, T791G, T796C, G807Del, A840G, A873G, A875G, C903T, T963G, C5810Del, A6101T, G6154T, G7421A, A7459C, and an insertion at nucleotide position 7322 having a sequence of SEQ ID NO: 179. In some embodiments, the XMRV polynucleotide is a detectable fragment thereof (e.g., at least about 10 or more contiguous nucleic acids containing at least one of the above nucleotide sequence changes). In some embodiments, the XMRV polynucleotide has a nucleic acid sequence having at least about 95% sequence identity to a sequence described above. In some embodiments, the XMRV polynucleotide has a nucleic acid sequence having at least about 95% sequence identity to a sequence described above and having an XMRV associated function or activity. In some embodiments, the XMRV polynucleotide is a functional fragment of a sequence described above having an XMRV associated function or activity.

In some embodiments, the XMRV associated function or activity is encoding of an RNA active gammaretrovirus core encapsidation signal. In some embodiments, the XMRV associated function or activity is formation of XMRV virion particles. In some embodiments, the XMRV associated function or activity is stimulation of a cytokine or chemokine signature indicative of an immune response in a subject in vivo. In some embodiments, the XMRV associated function or activity is formation of anti-XMRV antibodies according to an in vivo humoral immune response in a subject. In some embodiments, the XMRV associated function or activity is similar, same, or greater ex vivo fitness compared to an XMRV control or strain according to a growth competition assay. In some embodiments, the XMRV associated function or activity is ability to infect a cell in a modified Derse assay. In some embodiments, the XMRV associated function or activity is reverse transcriptase activity. In some embodiments, the XMRV associated function or activity is an ability to immortalize or modify a phenotype of a primary cell or cell culture. In some embodiments, the XMRV associated function or activity is an ability to induce cell syncytia or cell death on exposure or infection of cultured primary cells or co-cultured indicator cells. In some embodiments, the XMRV associated function or activity is an ability to form plaques in cell culture on exposure or infection. In some embodiments, the XMRV associated function or activity is similar, same, or lower tissue culture infective dose (TCID₅ₒ) compared to an XMRV control or strain. In various embodiments, the XMRV associated function or activity can be a combination of any of the above.

Another aspect proves an isolated XMRV polypeptide.

In some embodiments, the isolated XMRV polypeptide is an Envelope polypeptide having an amino acid sequence according to SEQ ID NO: 160 and one or more amino acid sequence changes selected from the group consisting of H116L, G134Stop, an insertion between amino acid positions 517-518 having an amino acid sequence of SEQ ID NO: 180, E535K, D549A, and R568G. In some embodiments, the isolated XMRV envelope polypeptide is a detectable fragment (e.g., at least about 4 or more contiguous amino acids containing at least one of the above amino acid sequence changes) of a sequence described above. In some embodiments, the isolated XMRV envelope polypeptide is an amino acid sequence having at least about 95% sequence identity to a sequence described above. In some embodiments, the isolated XMRV envelope polypeptide is an amino acid sequence having at least about 95% sequence identity to a sequence described above and having an XMRV associated function or activity. In some embodiments, the isolated XMRV envelope polypeptide is a functional fragment of a sequence described above having an XMRV associated function or activity.

In some embodiments, the XMRV associated function or activity is an extracellular topological domain at amino acid positions 34-585. In some embodiments, the XMRV associated function or activity is a helical transmembrane region at amino acid positions 586-606. In some embodiments, the XMRV associated function or activity is a cytoplasmic topological domain at amino acid positions 607-640. In some embodiments, the XMRV associated function or activity is a receptor-binding domain at amino acid positions 32-237. In some embodiments, the XMRV associated function or activity is a fusion peptide region at amino acid positions 447-467. In some embodiments, the XMRV associated function or activity is an immunosuppression region at amino acid positions 513-529. In some embodiments, the XMRV associated function or activity is a coiled coil region at amino acid positions 490-510. In some embodiments, the XMRV associated function or activity is a CXXC motif at amino acid positions 311-314. In some embodiments, the XMRV associated function or activity is a CX6CC motif at amino acid positions 530-538. In some embodiments, the XMRV associated function or activity is a YXXL motif containing an endocytosis signal at amino acid positions 630-633. In some embodiments, the XMRV associated function or activity is a Pro-rich region at amino acid positions 234-283. In some embodiments, the XMRV associated function or activity is a cleavage site at amino acid position 444-445. In some embodiments, the XMRV associated function or activity is a cleavage site at amino acid position 624-625. In some embodiments, the XMRV associated function or activity is an ability for the Envelope polypeptide to be cleaved to a surface protein (SU), a transmembrane protein (TM), and an R-protein. In some embodiments, the XMRV associated function or activity is SU activity, TM activity, or R-peptide activity. In some embodiments, the XMRV associated function or activity is an association of a trimer of SU-TM heterodimers attached by a labile interchain disulfide bond. In some embodiments, the XMRV associated function or activity is stimulation of a cytokine or chemokine signature indicative of an immune response in a subject in vivo. In some embodiments, the XMRV associated function or activity is formation of anti-XMRV antibodies according to an in vivo humoral immune response in a subject. In various embodiments, the XMRV associated function or activity can be a combination of any of the above.

In some embodiments, the isolated XMRV polypeptide is a Gag-Pol polypeptide having an amino acid sequence according to SEQ ID NO: 161 and one or more amino acid sequence changes selected from the group consisting of K31G, K31R, V36I, a 7 amino acid deletion from aa126-146, a 7 amino acid deletion from aa132-152, G59S, V60I, P105L, S27P, K31R, S62P; K65N, K65N and a downstream reading frame change according to SEQ ID NO: 105, and H76R. In some embodiments, the isolated XMRV Gag-Pol polypeptide is a detectable fragment (e.g., at least about 4 or more contiguous amino acids containing at least one of the above amino acid sequence changes) of a sequence described above. In some embodiments, the isolated XMRV Gag-Pol polypeptide has at least about 95% sequence identity to a sequence described above. In some embodiments, the isolated XMRV Gag-Pol polypeptide has at least about 95% sequence identity to a sequence described above having an XMRV associated function or activity. In some embodiments, the isolated XMRV Gag-Pol polypeptide is a functional fragment of a sequence described above having an XMRV associated function or activity.

In some embodiments, the XMRV associated function or activity is a peptidase A2 domain at amino acid position 559-629. In some embodiments, the XMRV associated function or activity is a reverse transcriptase domain at amino acid position 739-930. In some embodiments, the XMRV associated function or activity is an RNase H domain at amino acid position 1172-1318. In some embodiments, the XMRV associated function or activity is an integrase catalytic domain at amino acid position 1442-1600. In some embodiments, the XMRV associated function or activity is a CCHC-type domain at amino acid position 500-517. In some embodiments, the XMRV associated function or activity is a coiled coil at amino acid position 436-476. In some embodiments, the XMRV associated function or activity is a PTAP/PSAP motif at amino acid position 109-112. In some embodiments, the XMRV associated function or activity is a LYPX(n)L motif at amino acid position 128-132. In some embodiments, the XMRV associated function or activity is a PPXY motif at amino acid position 161-164. In some embodiments, the XMRV associated function or activity is a Pro-rich region at amino acid position 71-191. In some embodiments, the XMRV associated function or activity is or Pro-rich region at amino acid position 71-168. In some embodiments, the XMRV associated function or activity is a protease active site at amino acid position 564. In some embodiments, the XMRV associated function or activity is a magnesium metal binding catalytic site for reverse transcriptase activity at amino acid positions 807, 881, or 882. In some embodiments, the XMRV associated function or activity is a magnesium metal binding site for RNase H activity at amino acid positions 1181, 1219, 1240, or 1310. In some embodiments, the XMRV associated function or activity is a magnesium metal binding catalytic site for integrase activity at amino acid positions 1453 or 1512. In some embodiments, the XMRV associated function or activity is a cleavage site by viral protease p14 at amino acid positions 129-130, 213-214, 476-477, 532-533, 657-658, or 1328-1329. In some embodiments, the XMRV associated function or activity is an ability for the Gag-Pol polypeptide to be cleaved to a matrix protein p15, a RNA-binding phosphoprotein p12, a capsid protein p30, a nucleocapsid protein p10, a protease p14, a reverse transcriptase/ribonuclease H, and an integrase p46. In some embodiments, the XMRV associated function or activity is matrix protein p15 activity. In some embodiments, the XMRV associated function or activity is RNA-binding phosphoprotein p12 activity. In some embodiments, the XMRV associated function or activity is capsid protein p30 activity. In some embodiments, the XMRV associated function or activity is nucleocapsid protein p10 activity. In some embodiments, the XMRV associated function or activity is protease p14 activity. In some embodiments, the XMRV associated function or activity is reverse transcriptase/ribonuclease H activity. In some embodiments, the XMRV associated function or activity is integrase p46 activity. In some embodiments, the XMRV associated function or activity is stimulation of a cytokine or chemokine signature indicative of an immune response in a subject in vivo. In some embodiments, the XMRV associated function or activity is formation of anti-XMRV antibodies according to an in vivo humoral immune response in a subject. In various embodiments, the XMRV associated function or activity can be a combination of any of the above.

Another aspect provides a method of detecting a strain of XMRV in a sample, In some embodiments, the method includes detecting presence, absence, or quantity of an XMRV polynucleotide or polypeptide described above, or an immune response of a subject (e.g., production of an anti-XMRV antibody) thereto, in the sample.

In some embodiments, the sample is selected from a blood sample, a serum sample, a plasma sample, a cerebrospinal fluid sample, or a solid tissue sample. In some embodiments, the sample includes fibroblasts, endothelial cells, peripheral blood mononuclear cells, or haematopoietic cells, or a combination thereof.

In some embodiments, detecting presence, absence, or quantity of an XMRV strain in a sample includes contacting the sample and at least one probe that binds to at least one XMRV strain polypeptide, or detectable fragment thereof, under conditions sufficient for formation of a complex comprising the at least one probe and the least one polypeptide or fragment if present in the sample; and detecting presence, absence or quantity of the complex comprising the at least one probe and the at least one polypeptide or fragment. In some embodiments of probe-based detection, the at least one probe is a polyclonal antibody, a monoclonal antibody, an Fab fragment an antibody, an antigen-binding fragment of an antibody, an aptamer, or an avimer. In some embodiments of probe-based detection, the at least one probe is an anti gp 55 Env antibody, monoclonal antibody MAb 7C10, a monclonal antibody against p30 gag, or a polyclonal antibody against mouse xenotropic virus.

In some embodiments, probe-based detection includes at least one of an immunoprecipitation assay, an ELISA, a radioimmunoassay, a Western blot assay or a flow cytometry assay. In some embodiments, probe-based detection includes contacting the sample and the at least one probe comprises contacting the sample with a solid surface that binds the at least one XMRV polypeptide and subsequently contacting the surface with the at least one probe. In some embodiments, probe-based detection includes contacting the sample with a solid surface that binds the at least one XMRV polypeptide, subsequently contacting the surface with the at least one probe, and quantifying the at least one probe bound to the surface, wherein the solid surface is selected from the group consisting of a plate, a bead, a dip stick, a test strip, membrane and a microarray. In some embodiments of probe-based detection, the at least one probe includes a label; detecting presence, absence or quantity of a complex comprises quantifying the label; and the label is selected from the group consisting of a radioisotope, a chromogen, a chromophore, a fluorophore, a fluorogen, an enzyme, a quantum dot and a resonance light scattering particle. In some embodiments, probe-based detection includes contacting the complex and at least one secondary probe and detecting presence, absence or quantity of the at least one secondary probe, wherein at least one secondary probe binds the at least one probe or the at least one XMRV polypeptide.

In some embodiments, detecting presence, absence, or quantity of an XMRV strain in a sample includes a serocoversion assay. In some embodiments, serocoversion-based detection includes contacting the sample and at least one XMRV antigen under conditions sufficient for formation of a complex between the at least one XMRV antigen and an immunopeptide specific for an XMRV strain if the immunopeptide is present in the sample; and detecting presence, absence or quantity of the complex comprising the XMRV antigen and the anti-XMRV immunopeptide; wherein the XMRV antigen comprises the XMRV polynucleotide or polypeptide, or a fragment thereof.

In some embodiments, serocoversion-based detection includes contacting the complex comprising the XMRV antigen and the anti-XMRV immunopeptide of the sample with at least one probe directed against a serum retroviral immunopeptide or the XMRV antigen under conditions sufficient for formation of an complex comprising the at least one probe and the XMRV immunopeptide or the XMRV antigen; and detecting presence, absence or quantity of the probe. In some embodiments, serocoversion-based detection includes contacting the sample and at least one XMRV antigen comprises contacting the sample with a solid surface comprising a bound at least one XMRV antigen and detecting presence, absence or quantity of the complex comprising the XMRV antigen and the anti-XMRV immunopeptide. In some embodiments, serocoversion-based detection includes contacting the sample with a solid surface comprising a bound at least one XMRV antigen, contacting the surface with at least one probe directed against a serum retroviral immunopeptide under conditions sufficient for formation of an complex comprising the at least one probe and the XMRV immunopeptide, and detecting presence, absence or quantity of the probe, wherein the solid surface is selected from the group consisting of a plate, a bead, a dip stick, a test strip, membrane and a microarray. In some embodiments of serocoversion-based detection, the at least one XMRV antigen comprises a contiguous sequence of at least about 4 amino acids of the XMRV polypeptide comprising at least one of the amino acid sequence changes discussed above.

In some embodiments, detecting presence, absence, or quantity of an XMRV strain in a sample includes a nucleic acid-based assay. In some embodiments, nucleic acid-based detection includes contacting the sample and at least one nucleobase polymer under conditions sufficient for hybridization to occur between the at least one nucleobase polymer and a polynucleotide of a XMRV strain, or complement thereof, if present in the sample; and detecting presence, absence or quantity of a hybridization complex comprising the nucleobase polymer and the XMRV polynucleotide, or complement thereof wherein the at least one nucleobase polymer comprises a sequence that hybridizes to a nucleic acid sequence comprising at least about 10 contiguous nucleotides of a polynucleotide of an XMRV strain, or complement thereof.

In some embodiments of nucleic acid-based detection, the at least one nucleobase polymer comprises a sequence that hybridizes to a nucleic acid sequence comprising at least about 10 contiguous nucleotides of an XMRV polynucleotide comprising at least one of the nucleic acid sequence changes, or complement thereof. In some embodiments of nucleic acid-based detection, the conditions sufficient for hybridization to occur consists of high stringency hybridization conditions. In some embodiments of nucleic acid-based detection, the nucleobase polymer comprises DNA, RNA, or a nucleic acid analogue. In some embodiments of nucleic acid-based detection, the nucleobase polymer further comprises a label selected from the group consisting of a radioisotope, a chromogen, a chromophore, a fluorophore, a fluorogen, an enzyme, a quantum dot and a resonance light scattering particle, and detecting presence, absence or quantity of the hybridization complex comprises detecting presence, absence or quantity of the label. In some embodiments, nucleic acid-based detection includes a hybridization assay selected from the group consisting of a Southern hybridization assay, a Northern hybridization assay, a dot-blot hybridization assay, a slot-blot hybridization assay, a Polymerase Chain Reaction (PCR) assay and a flow cytometry assay. In some embodiments, nucleic acid-based detection includes a quantitative real time polymerase chain reaction assay.

In some embodiments, methods include correlating the presence, absence, or quantity of the XMRV strain with an XMRV-related disease or condition; wherein the sample is a sample of a subject. In some embodiments, the subject has, is suspected of having, or is at risk for developing an XMRV-related disease or condition. In some embodiments, the subject exhibits signs or symptoms of an XMRV-related disease or condition. In some embodiments, the XMRV-related disease or condition is selected from the group consisting of prostate cancer, Chronic Fatigue Syndrome, autism, autism spectrum disorders, Gulf War Syndrome, Multiple Sclerosis, Amyotrophic Lateral Sclerosis (ALS), Parkinson's disease, Niemann-Pick Type C Disease, fibromyalgia, chronic Lyme disease, non-epileptic seizures, thymoma, myelodysplasia, Immune Thrombocytopenic Purpura, Mantle Cell Lymphoma, and Chronic Lymphocytic Leukemia lymphoma.

In some embodiments, methods include selecting or modifying a treatment on the basis of detection of the presence, absence, or quantity of an XMRV strain in a sample of the subject. In some embodiments, methods include administering to the subject a therapeutically effective amount of an anti-viral compound if an XMRV strain is detected.

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 is a cartoon diagram of the XMRV genome, the HTLV genome, and the HIV1 genome, showing coding regions and non-coding regions including LTRs.

FIG. 2 is a sequence alignment showing similarities and differences between matrix protein sequences of XMRV and related viruses.

FIG. 3 is a series of line plots showing intracellular staining for XMRV Env using the SFFV env moAb (darker line, i.e., line shifted right at no AZT day 3) or isotype control (lighter line) for separated PBMC unactivated at time 0 or PHA/IL-2 activated in the presence and absence of 50 nM AZT for three days.

FIG. 4 is an alignment between the N-terminal regions of the Env protein of Spleen Focus-Forming Virus (SFFV, the top lines of the text), and XMRV (bottom lines of text). Bold font indicates differences between the two sequences.

FIG. 5 is a phylogenetic tree showing the relatedness of the three XMRV subgroups.

FIG. 6 is a phylogenetic tree showing the relatedness of the three XMRV subgroups.

FIG. 7 shows nucleotide variation in the sequences encoding matrix (“MA”) protein of XMRV sequences from the P subgroup. Nine XMRV clinical isolates (indicated by PBMC) are aligned relative to the reference sequence VP62. Nucleotide differences are indicated by boxes or shading.

FIG. 8 shows nucleotide variation in the sequences encoding matrix (“MA”) protein of XMRV compared to MLV sequences.

FIG. 9 is a phylogenetic tree showing the relationship between XMRV isolates and other gammaretroviruses.

FIG. 10 shows the results of a chromatogram from sequencing data from XMRV isolated from one infected subject. The chromatogram often shows two bases present at a single position, indicating that more than one distinct XMRV sequence is present within the clinical sample.

FIG. 11A is a cartoon showing sequence variation in the surface (“SU”) region of the Env protein between a clinical XMRV isolate (WPI-1104), the XMRV reference strain VP62, Pm-MLV, P-MLV and X-MLV. FIG. 11B is a cartoon showing sequence variation in the Env protein in two sequences isolated from the same XMR-infected subject.

FIG. 12 is a phylogenetic tree showing the relatedness of XMRV sequences. It shows that some clinical isolates of XMRV are more similar to xenotropic MLVs; whereas other clinical isolates of XMRV are more similar to polytropic or modified polytropic (Pm) MLVs.

FIG. 12 is a figure showing that APOBEC3G (A3G) activity may cause modification in nucleotide sequences of XMRV during the course of infection. Two clinical isolates of XMRV, 1186-B and 1125-B are shown.

FIG. 13 is a Western blot showing that the XMRV isolates from FIG. 12 are able to produce a translatable SU protein.

FIG. 14 is a sequence alignment of five polynucleotide sequences isolated from XMRV-infected subjects, and the VP62 reference sequence. The sequenced region corresponds to bases 5792-6281 in ENV, as counted with reference to VP62 (SEQ ID NO:1).

FIG. 15 is a sequence alignment of eight polynucleotide sequences isolated from XMRV-infected subjects, and the VP62 reference sequence. The sequenced region corresponds to bases 7183-7504 in ENV, as counted with reference to VP62 (SEQ ID NO:1).

FIG. 16 is a sequence alignment of forty polynucleotide sequences isolated from XMRV-infected subjects, and the VP62 reference sequence. The sequenced region corresponds to bases 665-1018 in GAG, as counted with reference to VP62 (SEQ ID NO:1).

FIG. 17 is a sequence alignment of five polypeptide sequences isolated from XMRV-infected subjects, and the VP62 reference sequence along with sequences for VP42 (SEQ ID NO: 164) and VP35 (SEQ ID NO: 163). The sequenced region corresponds to bases 5792-6281 in ENV, as counted with reference to VP62 (SEQ ID NO:1).

FIG. 18 is a sequence alignment of eight polypeptide sequences isolated from XMRV-infected subjects, and the VP62 reference sequence. The sequenced region corresponds to bases 7183-7504 in ENV, as counted with reference to VP62 (SEQ ID NO:1).

FIG. 19 is a sequence alignment of forty polypeptide sequences isolated from XMRV-infected subjects, and the VP62 reference sequence. The sequenced region corresponds to bases 665-1018 in GAG, as counted with reference to VP62 (SEQ ID NO:1).

FIG. 20 phylogenetic tree showing the relationships between XMRV sequences and murine xenotropic retroviruses.

FIG. 21 is a cartoon diagram of sequences showing that SU sequences of viruses transmitted from the plasma of UK ME/CFS patients to LNCaP cells share homology with XMRV and not with polytropic MLV.

FIG. 22 is a cartoon diagram of sequences showing that clones from one subject have sequences that are more similar to polytropic MLV sequences than to VP62 sequences.

### DETAILED DESCRIPTION OF THE INVENTION

The present disclosure is based, at least in part, on the observation that Xenotropic Murine Leukemia Virus-Related Virus (XMRV) exhibits significant sequence heterogeneity between clinical isolates; and that subjects infected with XMRV exhibit varying clinical symptoms.

XMRV Strains

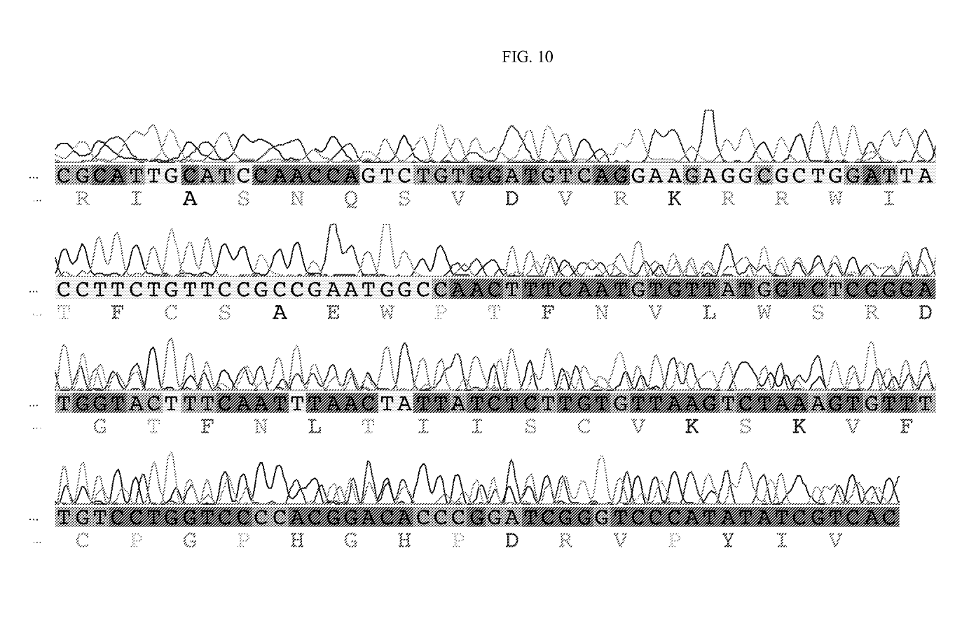

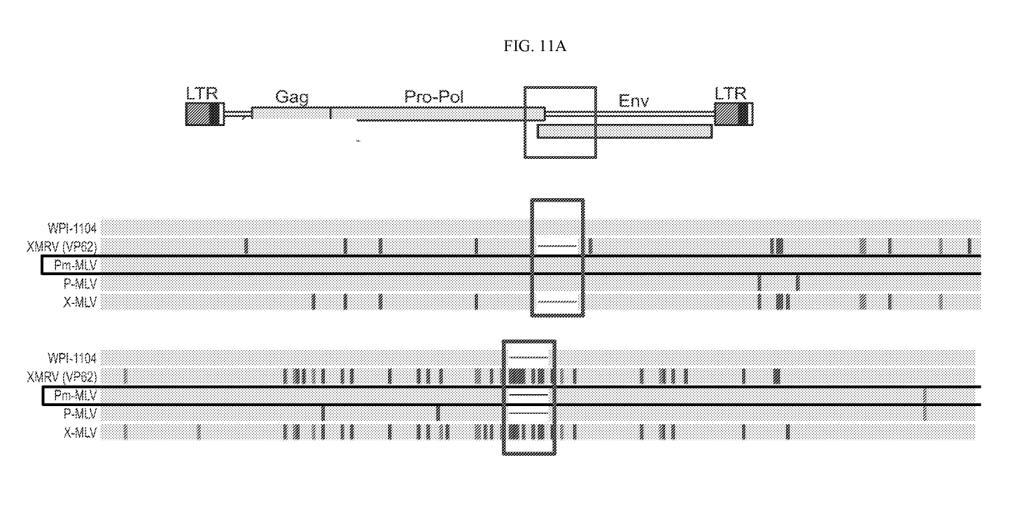

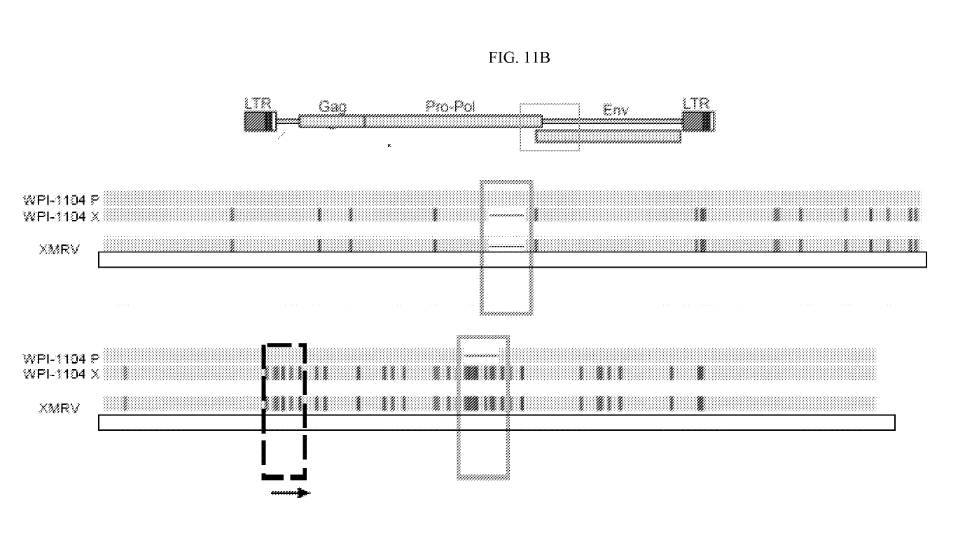

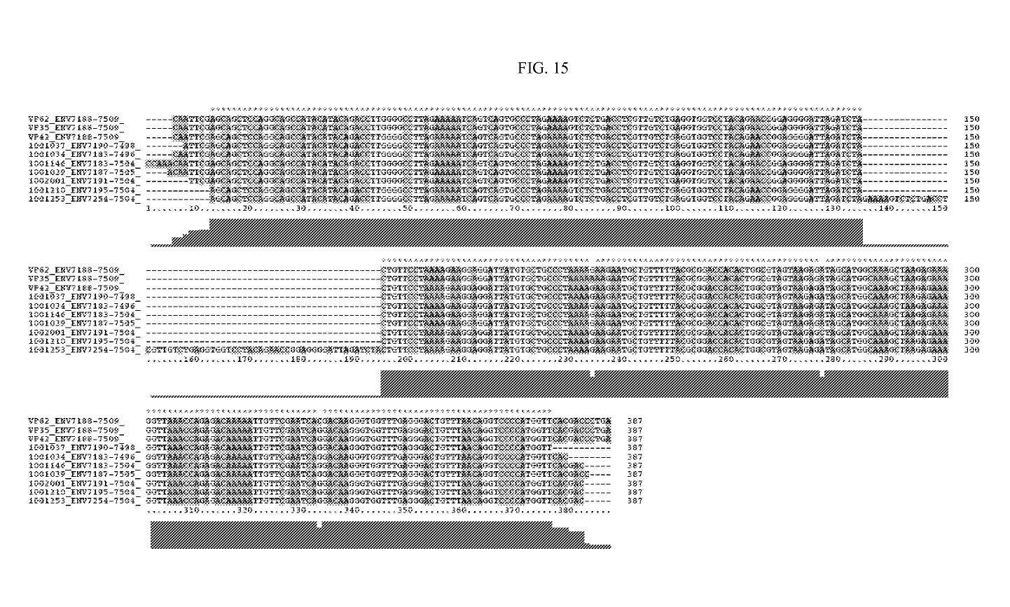

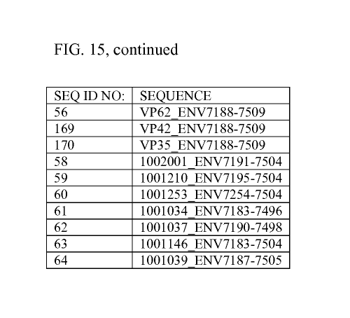

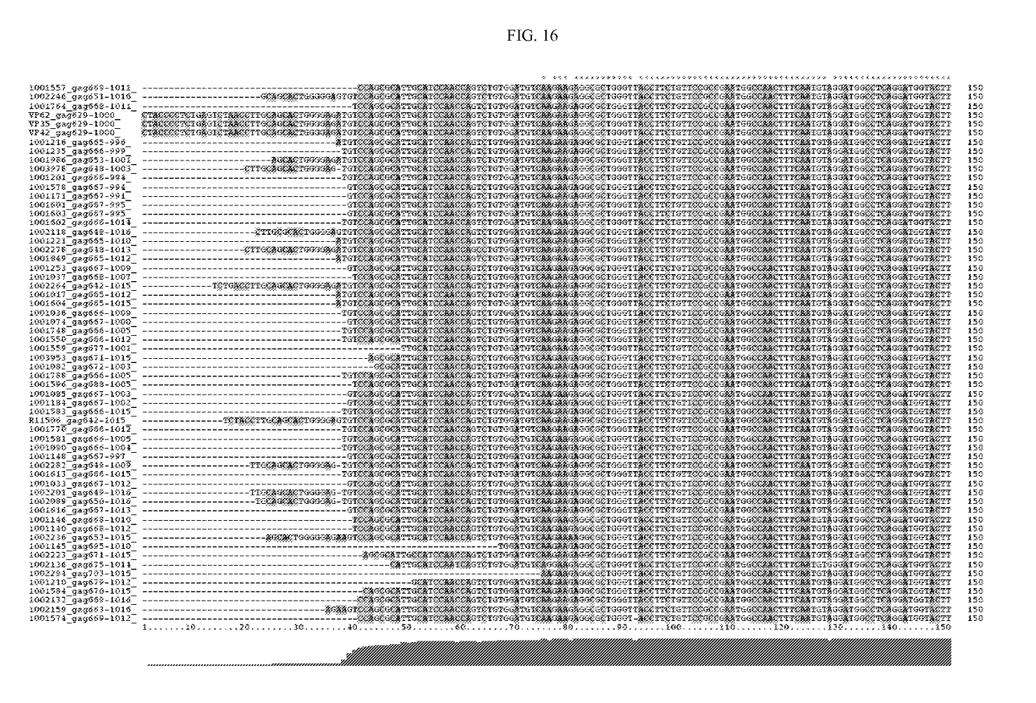

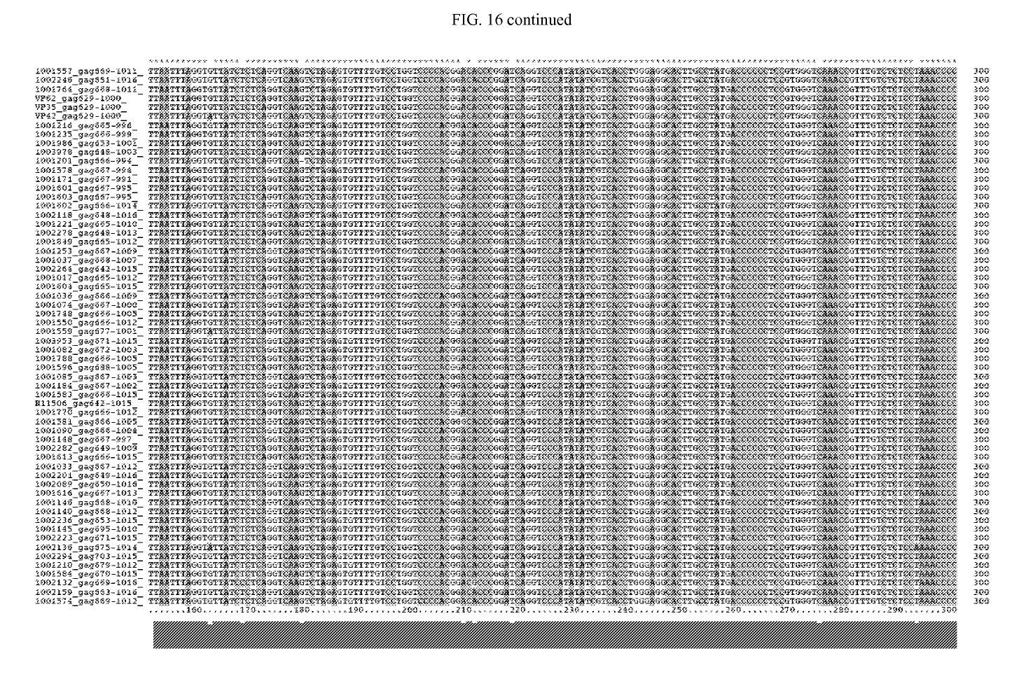

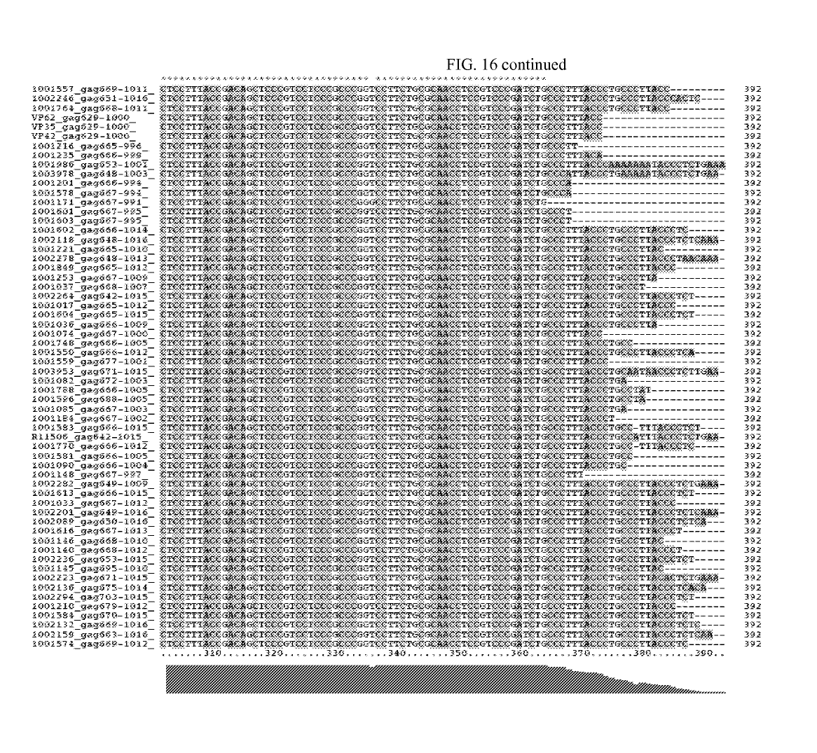

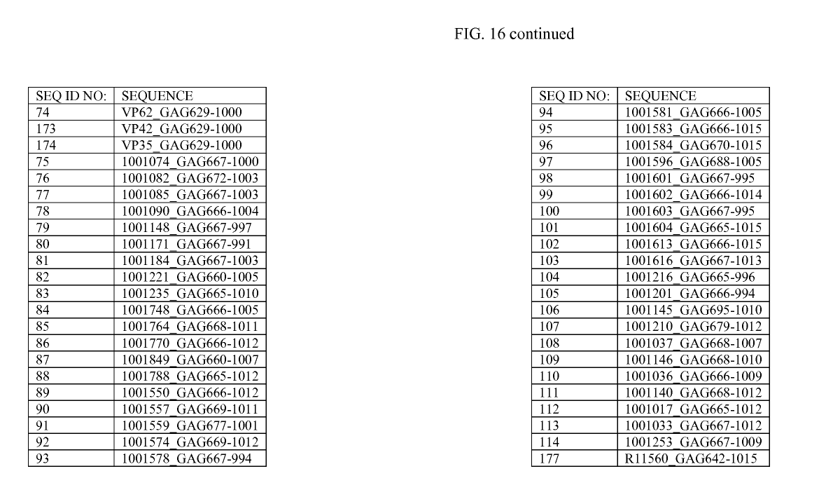

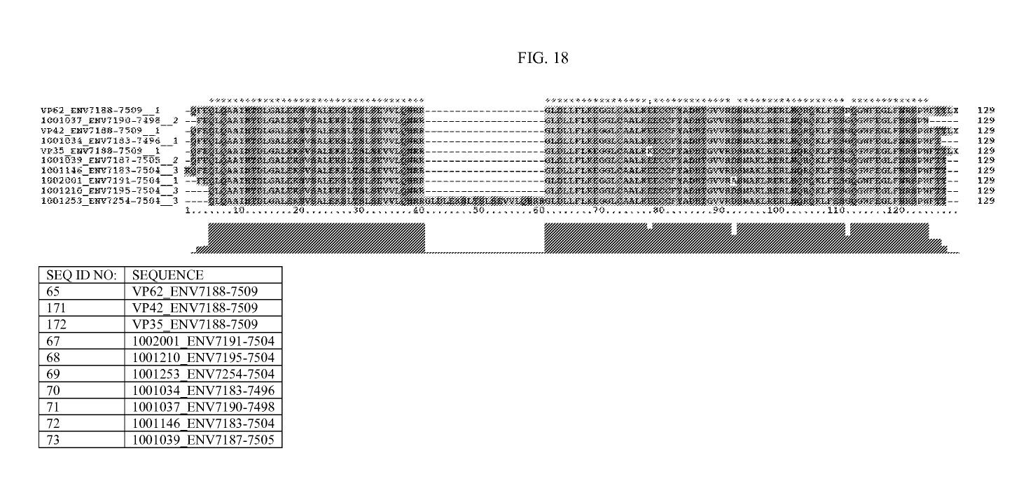

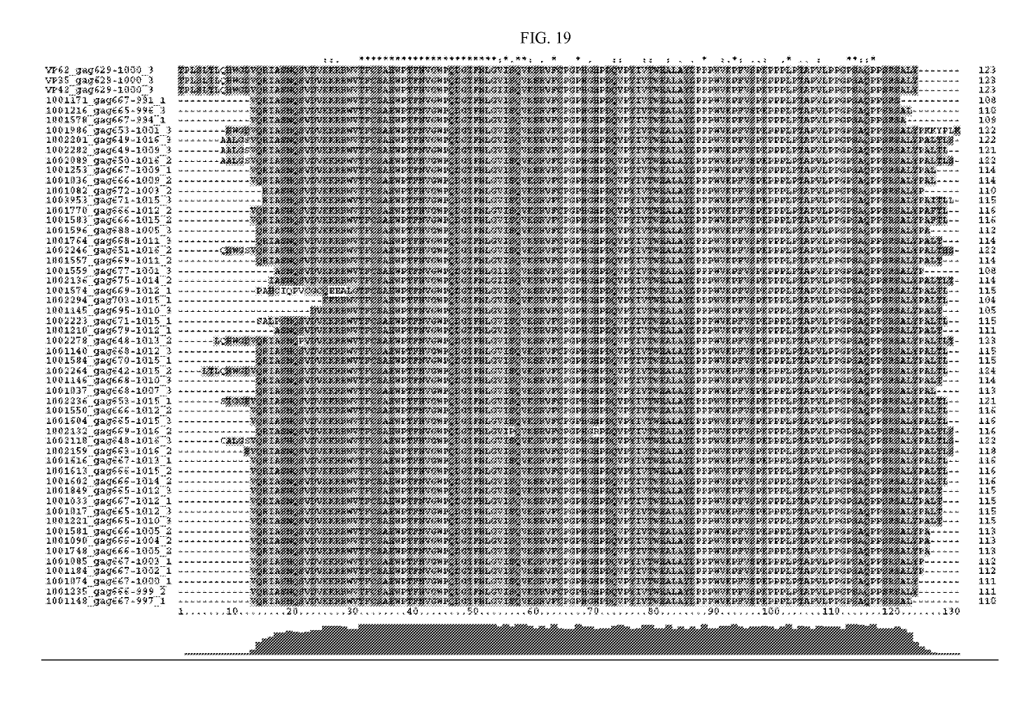

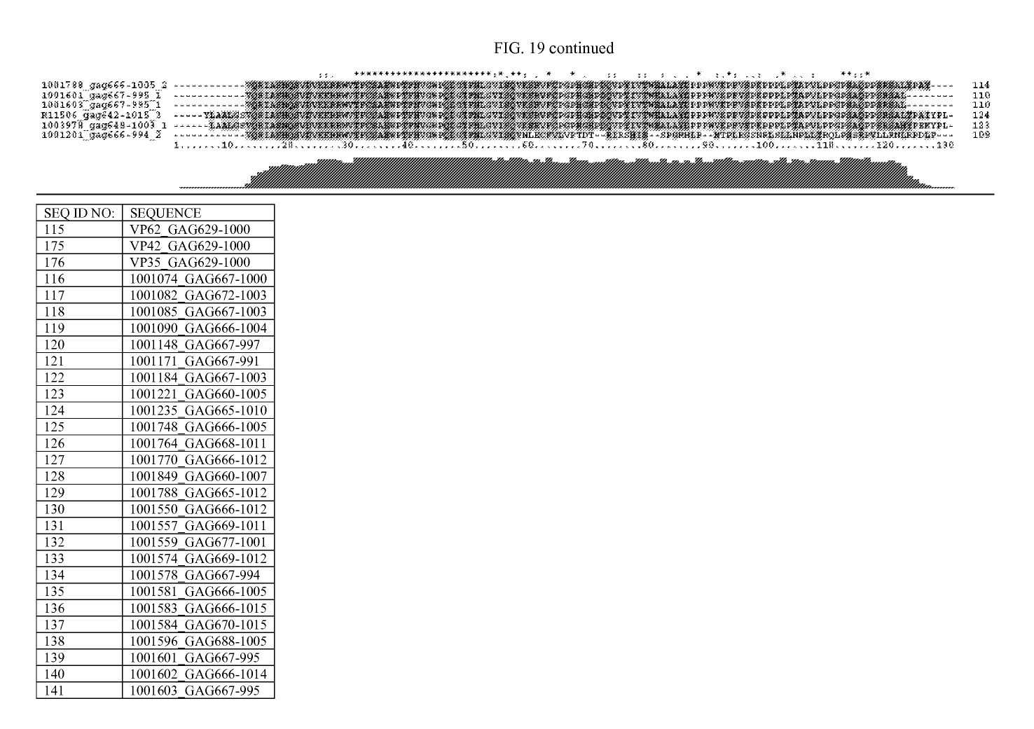

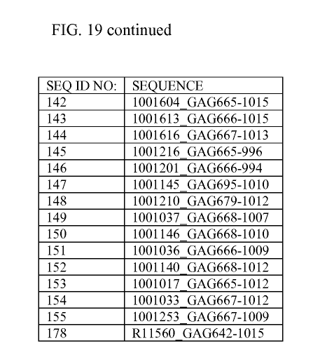

One aspect of the present disclosure provides isolated XMRV nucleic acid or polypeptide sequences. The present inventors have discovered multiple strains of XMRV isolates existing in nature, in the same or different subjects. The present inventors have also discovered that various XMRV strains can be categorized into distinctive subgroups. The present disclosure describes at least two distinct groups, identified herein as X-XMRV and P-XMRV. The P-XMRV group can include a modified P-XMRV, referred to herein as mP-XMRV. Various groups can be distinguished or defined by characteristic differences in their polynucleotide or polypeptide sequences (see e.g., TABLES 1-4 and FIGS. 5-11, 14-19). It has also been discovered that infection by multiple XMRV groups can occur in a single subject. For example, it is reported herein that a single individual can be infected with both P- and X-XMRV at the same time (see e.g., FIGS. 10-11).

The XMRV consensus sequence has been described previously (Urisman et al., PLOS Pathogens 2006 2(3):e25), Accession number DQ399707.1, and is referred to herein as VP62, or SEQ ID NO: 1. VP62 was identified from a clone reconstructed from nucleic acids isolated from prostate tumors. Accession number EF185282.1 (SEQ ID NO: 162) is an 8165 nucleotide sequence of VP62, while Accession number DQ399707.1 (SEQ ID NO: 1) is an 8185 nucleotide sequence of VP62. The reference sequence of SEQ ID NO: 1 corresponds to Accession number DQ399707.1. One of ordinary skill can determine corresponding positions of variations described herein with respect the other Accession sequence entry.

One aspect of the present disclosure provides sequences of XMRV that vary from the sequence of a “reference” VP62 sequence (see e.g., SEQ ID NO: 1). The variation can be detected and assessed by any methods known to ordinarily skilled artisans, including one or more of isolating viral polynucleotides, amplifying viral polynucleotides and sequencing viral polynucleotides. The variation can be detected by translating a polynucleotide sequence into a polypeptide sequence, and then comparing the translated polypeptide sequence to one or more other polypeptide sequences.

Polynucleotide Sequences of an XMRV Strain.

A polynucleotide of an XMRV strain can have a nucleic acid sequence according to reference VP62 (SEQ ID NO: 1) and one or more of the following nucleotide sequence changes: C80T, G90A, A96G, A97G, G111A, A137-157 deletion, T173C, G180A, G183A, C197T, C247T, C257T, C308T, C308G, C319T, C320T, T326C, A329G, C715T, T791G, A804G, T816Del, A856G, A665Del, T691G, G790A (potential hypermethylation site), T791G, T796C, G807Del, A840G, A873G, A875G, C903T, T963G, C5810Del, A6101T, G6154T, G7421A, A7459C, and an insertion at nucleotide position 7322 having a sequence of GAAAAGTCTCTGACCTCGTTGTCTGAGGTGGTCCTACAGAACCGGAGGGGAT TAGTCTA (SEQ ID NO: 179); or a functional fragment thereof.

A polynucleotide of an XMRV strain can have an XMRV associated function or activity and at least about 80% sequence identity to a sequence according to SEQ ID NO: 1 and having one or more nucleotide changes selected from C80T, G90A, A96G, A97G, G111A, A137-157 deletion, T173C, G180A, G183A, C197T, C247T, C257T, C308T, C308G, C319T, C320T, T326C, A329G, C715T, T791G, A804G, T816Del, A856G, A665Del, T691G, G790A (potential hypermethylation site), T791G, T796C, G807Del, A840G, A873G, A875G, C903T, T963G, C5810Del, A6101T, G6154T, G7421A, A7459C, and an insertion at nucleotide position 7322 having a sequence of GAAAAGTCTCTGACCTCGTTGTCTGAGGTGGTCCTACAGAACCGGAGGGGAT TAGTCTA (SEQ ID NO: 179); or a functional fragment thereof. For example, an XMRV strain can have at least two, at least three, at least four, at least five, at least sic, at least seven, at least eight, at least nine, or at least ten, or more, of nucleotide changes described herein.

For example, a polynucleotide of an XMRV strain can have an XMRV associated function or activity and at least about 85%, at least about 90%, at least about 91%, at least about 92%, at least about 93%, at least about 94%, at least about 95%, at least about 96%, at least about 97%, at least about 98%, or at least about 99% sequence identity to a sequence according to SEQ ID NO: 1 and having one or more (e.g., at least two, at least three, at least four, at least five, at least six, at least seven, at least eight, at least nine, or at least ten, or more) nucleotide changes selected from C80T, G90A, A96G, A97G, G111A, A137-157 deletion, T173C, G180A, G183A, C197T, C247T, C257T, C308T, C308G, C319T, C320T, T326C, A329G, C715T, T791G, A804G, T816Del, A856G, A665Del, T691G, G790A (potential hypermethylation site), T791G, T796C, G807Del, A840G, A873G, A875G, C903T, T963G, C5810Del, A6101T, G6154T, G7421A, A7459C, and an insertion at nucleotide position 7322 having a sequence of GAAAAGTCTCTGACCTCGTTGTCTGAGGTGGTCCTACAGAACCGGAGGGGAT TAGTCTA (SEQ ID NO: 179); or a functional fragment thereof.

As a further example, a polynucleotide of an XMRV strain can have an XMRV associated function or activity and at least about 95%, at least about 96%, at least about 97%, at least about 98%, or at least about 99% sequence identity to a sequence according to SEQ ID NO: 1 and having one or more (e.g., at least two, at least three, at least four, at least five, at least six, at least seven, at least eight, at least nine, or at least ten, or more) nucleotide changes selected from C80T, G90A, A96G, A97G, G111A, A137-157 deletion, T173C, G180A, G183A, C197T, C247T, C257T, C308T, C308G, C319T, C320T, T326C, A329G, C715T, T791G, A804G, T816Del, A856G, A665Del, T691G, G790A (potential hypermethylation site), T791G, T796C, G807Del, A840G, A873G, A875G, C903T, T963G, C5810Del, A6101T, G6154T, G7421A, A7459C, and an insertion at nucleotide position 7322 having a sequence of GAAAAGTCTCTGACCTCGTTGTCTGAGGTGGTCCTACAGAACCGGAGGGGAT TAGTCTA (SEQ ID NO: 179); or a functional fragment thereof.

A polynucleotide of an XMRV strain can be a functional fragment of a polynucleotide sequence disclosed herein. A functional fragment of an XMRV polynucleotide sequence can be an upstream or downstream truncated XMRV sequence, where the polynucleotide retains an XMRV associated function or activity, as described further herein, or the polynucleotide encodes a polypeptide having an XMRV associated function or activity, as described further herein. Polynucleotide or polypeptide function or activity of an XMRV strain can be as discussed further herein.

A detectable polynucleotide fragment of an XMRV strain disclosed herein can comprise at least about 10 contiguous nucleotides of a polynucleotide sequence described herein. For example, detectable polynucleotide fragment of an XMRV strain disclosed herein can comprise at least about 15, at least about 20, at least about 25, at least about 50, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, or at least about 1000, or more, contiguous nucleotides of a polynucleotide sequence described herein. A detectable polynucleotide fragment can have at least one (e.g., at least two, at least three, at least four, at least five, at least six, at least seven, at least eight, at least nine, or at least ten, or more) nucleic acid change described herein.

Polypeptide Sequences of an XMRV Strain.

Envelope.

An XMRV strain can have a polypeptide sequence according to reference VP62 Envelope polypeptide (SEQ ID NO: 160) and one or more of the following amino acid sequence changes: H116L, G134Stop, an insertion between amino acid positions 517-518 having a sequence of GLDLEKSLTSLSHVVLQNRR (SEQ ID NO: 180), E535K, D549A, and R568G, or a functional fragment thereof. For example, an Envelope polypeptide of an XMRV strain can have at least two, at least three, at least four, at least five, or at least six, or more, of amino acid changes described herein.

A polypeptide of an XMRV strain can have an XMRV associated function or activity and at least about 80% sequence identity to a polypeptide sequence according to reference VP62 Envelope polypeptide SEQ ID NO: 160 and one or more (e.g., at least two, at least three, at least four, at least five, or at least six, or more) of the following amino acid sequence changes: H116L, G134Stop, an insertion between amino acid positions 517-518 having a sequence of GLDLEKSLTSLSHVVLQNRR (SEQ ID NO: 180), E535K, D549A, and R568G, or a functional fragment thereof.

For example, a polypeptide of an XMRV strain can have an XMRV associated function or activity and at least about 85%, at least about 90%, at least about 91%, at least about 92%, at least about 93%, at least about 94%, at least about 95%, at least about 96%, at least about 97%, at least about 98%, or at least about 99% sequence identity to an amino acid sequence according to reference VP62 Envelope polypeptide SEQ ID NO: 160 and one or more (e.g., at least two, at least three, at least four, at least five, or at least six, or more) of the following amino acid sequence changes: H116L, G134Stop, an insertion between amino acid positions 517-518 having a sequence of GLDLEKSLTSLSHVVLQNRR (SEQ ID NO: 180), E535K, D549A, and R568G, or a functional fragment thereof.

As a further example, a polypeptide of an XMRV strain can have an XMRV associated function or activity and at least about 95%, at least about 96%, at least about 97%, at least about 98%, or at least about 99% sequence identity to an amino acid sequence according to reference VP62 Envelope polypeptide SEQ ID NO: 160 and one or more (e.g., at least two, at least three, at least four, at least five, or at least six, or more) of the following amino acid sequence changes: H116L, G134Stop, an insertion between amino acid positions 517-518 having a sequence of GLDLEKSLTSLSHVVLQNRR (SEQ ID NO: 180), E535K, D549A, and R568G, or a functional fragment thereof.

Gag-Pol.

A polypeptide of an XMRV strain can have a polypeptide sequence according to reference VP62 Gag-Pol polypeptide (SEQ ID NO: 161) and one or more of the following amino acid sequence changes: K31G, K31R, V36I, 7 amino acid deletion from aa126-146, 7 amino acid deletion from aa132-152, G59S, V60I, P105L, S27P, K31R, S62P; K65N, K65N and a downstream reading frame change according to SEQ ID NO: 105, and H76R; or a functional fragment thereof. For example, a Gag-Pol polypeptide of an XMRV strain can have at least two, at least three, at least four, at least five, or at least six, or more, of amino acid changes described herein.

For example, a polypeptide of an XMRV strain can have an XMRV associated function or activity and at least about 85%, at least about 90%, at least about 91%, at least about 92%, at least about 93%, at least about 94%, at least about 95%, at least about 96%, at least about 97%, at least about 98%, or at least about 99% sequence identity to an amino acid sequence according to reference VP62 Gag-Pol polypeptide (SEQ ID NO: 161) and one or more (e.g., at least two, at least three, at least four, at least five, at least six, at least seven, at least eight, at least nine, or at least ten, or more) of the following amino acid sequence changes: K31G, K31R, V36I, 7 amino acid deletion from aa126-146, 7 amino acid deletion from aa132-152, G59S, V60I, P105L, S27P, K31R, S62P; K65N, K65N and a downstream reading frame change according to SEQ ID NO: 105, and H76R; or a functional fragment thereof.

As a further example, a polypeptide of an XMRV strain can have an XMRV associated function or activity and at least about 95%, at least about 96%, at least about 97%, at least about 98%, or at least about 99% sequence identity to an amino acid sequence according to reference VP62 Gag-Pol polypeptide (SEQ ID NO: 161) and one or more (e.g., at least two, at least three, at least four, at least five, at least six, at least seven, at least eight, at least nine, or at least ten, or more) of the following amino acid sequence changes: K31G, K31R, V36I, 7 amino acid deletion from aa126-146, 7 amino acid deletion from aa132-152, G59S, V60I, P105L, S27P, K31R, S62P; K65N, K65N and a downstream reading frame change according to SEQ ID NO: 105, and H76R; or a functional fragment thereof.

A polypeptide of an XMRV strain can be a functional fragment of a polypeptide sequence disclosed herein. A functional fragment of an XMRV polypeptide sequence can be an upstream or downstream truncated XMRV polypeptide sequence, where the polypeptide retains an XMRV associated function or activity, as described further herein. Polypeptide function or activity of an XMRV strain can be as discussed further herein.

A detectable polypeptide fragment of an XMRV strain disclosed herein can comprise at least about 4 contiguous amino acids of a polypeptide sequence described herein. For example, detectable polypeptide fragment of an XMRV strain disclosed herein can comprise at least about 6, at least about 8, at least about 10, at least about 15, at least about 20, at least about 30, at least about 40, at least about 50, at least about 60, at least about 70, at least about 80, at least about 90, at least about 100, at least about 150, at least about 200, at least about 250, at least about 300, at least about 350, at least about 400, at least about 450, at least about 500, at least about 600, at least about 700, at least about 800, at least about 900, or at least about 100, or more, contiguous amino acids of a polypeptide sequence described herein. A detectable polypeptide fragment can have at least one (e.g., at least two, at least three, at least four, at least five, at least six, at least seven, at least eight, at least nine, or at least ten, or more) amino acid change described herein.

The present inventors have discovered that there is variation in the XMRV viral RNA that is expressed in peripheral blood mononuclear cells (PBMCs). Findings described herein show more sequence diversity between XMRV viral polynucleic acids than has been previously reported. Described herein are at least two subgroups of XMRV: subgroup X and subgroup P. The X subgroup of XMRV (X-XMRV) is shown herein to be closely related to known XMRV sequences and X-MLVs, but does have some nucleotide substitutions relative to known reference sequences, such as VP62 (SEQ ID NO: 1). The P subgroup of XMRV (P-XMRV) is shown herein to be closely related to P-MLVs and Pm-MLVs and has been discovered to have several specific differences. For example, in MA sequences, P-XMRV differs from known XMRV sequences at a number of nucleotides, although it is highly conserved with other XMRV sequences at the amino acid level. As another example, in SU sequences, P-XMRV cannot be detected by PCR primers based on X-XMRV-type sequences, further suggesting that P-XMRV SU sequences are different from X-XMRV sequences.

XMRV has a 24-nt deletion in the glycoGag region of its genome, relative to any other known exogenous MuLV. This 24-nt deletion encompasses a stop codon that is 53 amino acids downstream from the alternative translational start site. While no other MuLV is known to share the same 24-nt deletion as XMRV, a shorter deletion of nine nucleotides internal to the 24-nt deletion is present in the genomes of several non-ecotropic MuLV proviruses. In cultured cells, the glycoGag region is not essential for viral replication, and lesions in this same region have been associated with variations in pathogenic properties in vivo. For example, an alteration in ten nucleotides affecting five residues in the N-terminal peptide of glycoGag was found to be responsible for a 100-fold difference in the frequency of neuroinvasion observed between CasFrKP and CasFrKP41 MuLV strains.

Table 1 identifies variation in XMRV sequences, and shows which amino acid residue/positions characterize both X- and P-XMRV groups (see e.g., Examples 4-8).

TABLE 1
Nucleotide changes identified in clinical isolates
of XMRV, with reference to sequence numbering of VP62,
Accession number DQ399707.1 (SEQ ID NO: 1) and Accession
number EF185282.1 (SEQ ID NO: 162).

| Location in | Location in | | |
|---|---|---|---|
| SEQ ID NO: 1 | SEQ ID NO: 162 | Groups | AA change |
| C80T | C75T | mP | |
| G90A | G85A | mP | |
| A96G | A91G | mP | K31G |
| A97G | A92G | X, mP, P | K31R |
| G111A | G106A | P | V36I |
| A137-157 deletion | A132-152 deletion | mP | 7 amino acid |
| | | | deletion* |
| T173C | T168C | P | |
| G180A | G175A | P | G59S |
| #G183A | #G178A | X, mP, P | V60I |
| C197T | C192T | P | |
| C247T | C242T | P | |
| C257T | C252T | P | |
| C308T | C303T | P | |
| C308G | C303G | mP | |
| C319T | C314T | mP | P105L |
| C320T | C315T | mP, P | |
| T326C | T321C | mP, P | |
| A329G | A324G | X, mP, P | |

Amino acid changes determined with respect to alignment SEQ ID NO: 162.
*Due to direct repeat in this region (ATGGCC), deletion could be from 126-146 or from 132-152.
#place where VP42, EK1 and EK2 have same substitutions relative to the other published.

Lys31Arg is present in VP35 (SEQ ID NO: 163) while VP42 (SEQ ID NO: 164) has Lysine at position 31. At position 60, VP62, VP35 are both Valine, while X, mP, and P are Isoleucine. The 21 base pair deletion at A132-152, resulting in a deletion of seven amino acid residues, is predicted (based on similarity with crystal structure of the MA in Mp-MLV) to be located in a short 3₁₀ helix located between helices 2 and 3 (see Riffel 2002 Structure 10(12), 1627-1636).

Table 2 identifies sequence variation in strains of XMRV sequences from clinical samples (see Example 9).

TABLE 2
Nucleotide and Amino Acid Variation in XMRV Strains
compared to VP62 (SEQ ID NO: 1).

| Nucleotide Change, | | |
|---|---|---|
| written as (VP62 nt) | | |
| (sequence position) | Resulting amino | |
| (differing nucleotide | acid change, | Subject Number |
| in clinical isolate) | if any (protein) | (for AA change) |
| C715T | | |
| T791G | | |
| A804G | | |
| T816Del | | |
| A856G | | |
| A665Del | | |
| T691G | S27P (Gag) | 1002278 |
| A704G | K31R (Gag) | 1002136 |
| G790A (potential | | |
| hypermethylation site) | | |
| T791G | S62P (Gag) | 1002132 |
| T796C | | |
| G807Del | K65N followed by | 1001201 |
| | complete reading frame | |
| | change but not including | |
| | a stop codon (Gag) | |
| A840G | H76R (Gag) | 1002132 |
| A873G | | |
| A875G | | |
| C903T | | |
| T963G | | |
| C5810Del | | |
| A6101T | H116L (Env) | 1000873 |
| G6154T | G134Stop (Env) | 1000888 |
| insertion from nt | additional amino acids | 1001253 |
| 7322 to 7381: | (517 of VP62 Env): | |
| GAAAAGTCTCTGACCTCGT | GLDLEKSLTSLSHVV | |
| TGTCTGAGGTGGTCCTACA | LQNRR (518 of VP62 Env) | |
| GAACCGGAGGGGATTAGTC | (SEQ ID NO: 180) | |
| TA (SEQ ID NO: 179) | | |
| G7421A | E535K (Env) | VP35 |
| A7459C | D549A (Env) | 1002001 |
| C7515G | R568G (Env) | 1000889, VP35, |
| | | 1001039, 1001034, |
| | | 10011146, VP42, |
| | | 1001037, 1002001, |
| | | 1001210, 1001253 |

Tables 3-5 provides variation found in XMRV polynucleotide and polypeptide sequences (see e.g., Example 9). Subject number 1001253 was identified as having a P-type XMRV (SEQ ID NOS: 60, 69, 114, 155).

TABLE 3
Variation in XMRV sequences.

| | | Position of sequence from |
|---|---|---|
| | Source of sequence | subject relative to VP62 |
| SEQ ID NO: | (subject number) | reference sequence |
| 44 | VP62 reference | ENV5797-6286 |
| 165 | VP42 reference | ENV5797-6286 |
| 166 | VP35 reference | ENV5797-6286 |
| 45 | 1000875 | ENV5811-6201 |
| 46 | 1000871 | ENV5797-6286 |
| 47 | 1000888 | ENV5815-6105 |
| 48 | 1000867 | ENV5803-6173 |
| 49 | 1000873 | ENV5803-6110 |
| 50 | VP62 reference (peptide) | ENV5792-6281 |
| 167 | VP42 reference (peptide) | ENV5797-6286 |
| 168 | VP35 reference (peptide) | ENV5797-6286 |
| 51 | 1000875 (peptide) | ENV5811-6201 |
| 52 | 1000871 (peptide) | ENV5792-6286 |
| 53 | 1000888 (peptide) | ENV5815-6105 |
| 54 | 1000867 (peptide) | ENV5803-6173 |
| 55 | 1000873 (peptide) | ENV5803-6110 |
| 56 | VP62 reference | ENV7188-7509 |
| 169 | VP42 reference | ENV7188-7509 |
| 170 | VP35 reference | ENV7188-7509 |
| 57 | 1000889 | ENV7191-7333 |
| 58 | 1002001 | ENV7191-7504 |
| 59 | 1001210 | ENV7195-7504 |
| 60 | 1001253 | ENV7254-7504 |
| 61 | 1001034 | ENV7183-7496 |
| 62 | 1001037 | ENV7190-7498 |
| 63 | 1001146 | ENV7183-7504 |
| 64 | 1001039 | ENV7187-7505 |
| 65 | VP62 reference (peptide) | ENV7188-7509 |
| 171 | VP42 reference (peptide) | ENV7188-7509 |
| 172 | VP35 reference (peptide) | ENV7188-7509 |
| 66 | 1000889 (peptide) | ENV7191-7333 |
| 67 | 1002001 (peptide) | ENV7191-7504 |
| 68 | 1001210 (peptide) | ENV7195-7504 |
| 69 | 1001253 (peptide) | ENV7254-7504 |
| 70 | 1001034 (peptide) | ENV7183-7496 |
| 71 | 1001037 (peptide) | ENV7190-7498 |
| 72 | 1001146 (peptide) | ENV7183-7504 |
| 73 | 1001039 (peptide) | ENV7187-7505 |
| 74 | VP62 reference | GAG629-1000 |
| 173 | VP42 reference | GAG629-1000 |
| 174 | VP35 reference | GAG629-1000 |
| 75 | 1001074 | GAG667-1000 |
| 76 | 1001082 | GAG672-1003 |
| 77 | 1001085 | GAG667-1003 |
| 78 | 1001090 | GAG666-1004 |
| 79 | 1001148 | GAG667-997 |
| 80 | 1001171 | GAG667-991 |
| 81 | 1001184 | GAG667-1003 |
| 82 | 1001221 | GAG660-1005 |
| 83 | 1001235 | GAG665-1010 |
| 84 | 1001748 | GAG666-1005 |
| 85 | 1001764 | GAG668-1011 |
| 86 | 1001770 | GAG666-1012 |
| 87 | 1001849 | GAG660-1007 |
| 88 | 1001788 | GAG665-1012 |
| 89 | 1001550 | GAG666-1012 |
| 90 | 1001557 | GAG669-1011 |
| 91 | 1001559 | GAG677-1001 |
| 92 | 1001574 | GAG669-1012 |
| 93 | 1001578 | GAG667-994 |
| 94 | 1001581 | GAG666-1005 |
| 95 | 1001583 | GAG666-1015 |
| 96 | 1001584 | GAG670-1015 |
| 97 | 1001596 | GAG688-1005 |
| 98 | 1001601 | GAG667-995 |
| 99 | 1001602 | GAG666-1014 |
| 100 | 1001603 | GAG667-995 |
| 101 | 1001604 | GAG665-1015 |
| 102 | 1001613 | GAG666-1015 |
| 103 | 1001616 | GAG667-1013 |
| 104 | 1001216 | GAG665-996 |
| 105 | 1001201 | GAG666-994 |
| 106 | 1001145 | GAG695-1010 |
| 107 | 1001210 | GAG679-1012 |
| 108 | 1001037 | GAG668-1007 |
| 109 | 1001146 | GAG668-1010 |
| 110 | 1001036 | GAG666-1009 |
| 111 | 1001140 | GAG668-1012 |
| 112 | 1001017 | GAG665-1012 |
| 113 | 1001033 | GAG667-1012 |
| 114 | 1001253 | GAG667-1009 |
| 177 | R11560 | GAG642-1015 |
| 115 | VP62 reference (peptide) | GAG629-1000 |
| 175 | VP42 reference (peptide) | GAG629-1000 |
| 176 | VP35 reference (peptide) | GAG629-1000 |
| 116 | 1001074 (peptide) | GAG667-1000 |
| 117 | 1001082 (peptide) | GAG672-1003 |
| 118 | 1001085 (peptide) | GAG667-1003 |
| 119 | 1001090 (peptide) | GAG666-1004 |
| 120 | 1001148 (peptide) | GAG667-997 |
| 121 | 1001171 (peptide) | GAG667-991 |
| 122 | 1001184 (peptide) | GAG667-1002 |
| 123 | 1001221 (peptide) | GAG665-1010 |
| 124 | 1001235 (peptide) | GAG666-999 |
| 125 | 1001748 (peptide) | GAG666-1005 |
| 126 | 1001764 (peptide) | GAG668-1011 |
| 127 | 1001770 (peptide) | GAG661-1007 |
| 128 | 1001849 (peptide) | GAG666-1012 |
| 129 | 1001788 (peptide) | GAG666-1005 |
| 130 | 1001550 (peptide) | GAG666-1012 |
| 131 | 1001557 (peptide) | GAG669-1011 |
| 132 | 1001559 (peptide) | GAG677-1001 |
| 133 | 1001574 (peptide) | GAG669-1012 |
| 134 | 1001578 (peptide) | GAG667-994 |
| 135 | 1001581 (peptide) | GAG666-1005 |
| 136 | 1001583 (peptide) | GAG666-1015 |
| 137 | 1001584 (peptide) | GAG665-1007 |
| 138 | 1001596 (peptide) | GAG670-1015 |
| 139 | 1001601 (peptide) | GAG667-995 |
| 140 | 1001602 (peptide) | GAG666-1014 |
| 141 | 1001603 (peptide) | GAG667-995 |
| 142 | 1001604 (peptide) | GAG665-1015 |
| 143 | 1001613 (peptide) | GAG666-1015 |
| 144 | 1001616 (peptide) | GAG667-1013 |
| 145 | 1001216 (peptide) | GAG665-996 |
| 146 | 1001201 (peptide) | GAG666-994 |
| 147 | 1001145 (peptide) | GAG695-1010 |
| 148 | 1001210 (peptide) | GAG679-1012 |
| 149 | 1001037 (peptide) | GAG668-1007 |
| 150 | 1001146 (peptide) | GAG668-1010 |
| 151 | 1001036 (peptide) | GAG666-1009 |
| 152 | 1001140 (peptide) | GAG668-1012 |
| 153 | 1001017 (peptide) | GAG665-1012 |
| 154 | 1001033 (peptide) | GAG667-1012 |
| 155 | 1001253 (peptide) | GAG666-999 |
| 178 | R11560 | GAG642-1015 |

Numbering for all sequences refers to corresponding positions on the reference VP62 sequence (SEQ ID NO: 1). Peptide sequences were determined by in silico translation of the nucleotide sequence isolated from the same subject: nucleotide SEQ ID NOs: 44-49 correspond to peptide SEQ ID NOs: 50-55 respectively; nucleotide SEQ ID NOs: 56-64 correspond to peptide SEQ ID NOs: 65-73, respectively; and nucleotide SEQ ID NOs: 74-114 correspond to peptide SEQ ID NOs: 115-155, respectively.

TABLE 4
Additional XMRV Sequences

| | | Position of sequence from |
|---|---|---|
| | Source of sequence | subject relative to VP62 |
| SEQ ID NO: | (subject number) | reference sequence |
| 23 | VP62 reference | GAG |
| 24 | 11 | GAG |
| 25 | 10 | ENV5798-6105 |
| 26 | VP62 reference | |
| 27 | 17 | 5724-5940 |
| 28 | VP62 reference | ENV |
| 29 | 18 | ENV5814-5897 |
| 30 | | GAG |
| 31 | | GAG667-1015 |
| 32 | 4 | 5798-6168 |
| 33 | VP62 reference | ENV |
| 34 | 8 | ENV7185-7324 |
| 35 | | GAG |
| 36 | | GAG628-964 |
| 37 | 1 | ENV5806-6197 |
| 38 | WPI-1106 | |
| 39 | 1-23 | |
| 40 | WPI1138 | |
| 41 | 2-1 | |

TABLE 5
Variation in XMRV sequences.

| | | | | Chronic Fatigue Syndrome Cases | | |
|---|---|---|---|---|---|---|
| | | | | | | WPI-1104 |
| | Prostate Cancer Cells | | | | | (36- |
| | VP 62 | VP 42 | VP 35 | WPI-1106 | WPI-1178C | 1152; 5923- |
| nt (number) | (4-8174 nt) | (1-8186 nt) | (1-8186 nt) | (36-8144 nt) | (36-8144 nt) | 8147 nt) |
| 375 | | | A | | | |
| 450 | | | C | | | |
| 790 | | A | | | | |
| 1013 | | | T | | | |
| 1477 | | | G | | | |
| 1565 | | G | | | | |
| 1824 | | G | G | | | |
| 2413 | | | | A/G | | |
| 2416 | | | | | | |
| 2559 | | | A | | | |
| 2602 | | | A | | | |
| 2622 | | G | | | | |
| 4159 | | G | | | | |
| 4229 | | C deletion | | | | |
| 4236 | | G insertion | | | | |
| 4883 | | | | T | | |
| 4985 | | | | A | | |
| 5083 | | | T | | | |
| 5087 | | | A | | | |
| 5313 | | | | | | G |
| 5823 | | | | | C | |
| 5830 | | | | G | | |
| 6373 | | | | | G | |
| 6651 | | A | | | | |
| 7064 | | | G | | | |
| 7357 | | | A | | | |
| 7437 | | | | G | | |
| 7451 | | | | G | G | G |
| 7456 | | G | G | | | |
| 7692 | | | | | T insertion | |
| 7782 | | G insertion | G insertion | | G insertion | |

TABLE 5 identifies amino acid positions in the XMRV MA (gag) protein that are conserved in closely related gammaretroviruses.

TABLE 5
Amino acid substitutions of XMRV MA
found in other gammaretroviruses.

| aa change | aa identical to substitution |
|---|---|
| Lys (31)-Arg/Gly | FeLV, Fr-MLV, KoRV (Arg); none (Gly) |
| Val (36)-Ile | FeLV |
| Gly (59)-Ser | GaLV, KoRV, |
| Val (60)-Ile | AKV-MLV, Ampho-MLV, Cas-BrE, Fr-MLV, |
| | Mo-MLV, X-MLV |
| Pro (105)-Leu | AKV-MLV, X-MLV |

Accession numbers: AKV MLV (MLOCG), Amphotropic MLV (AF411814), Cas-BrE (X57540), FeLV (AF052723) Friend MLV (Fr-MLV) (NC 001362), GaLV (NC 001885), KoRV (QT9TTC2), Moloney-MLV (NC 001501), and xenotropic MLV (X-MLV)(EU035300).

XMRV Function

Described herein are polynucleotides or polypeptides of XMRV strains having a specified percentage sequence identity to a sequence described herein where such polynucleotides or polypeptides have an XMRV associated function or activity. Also described herein are functional fragments of polynucleotides or polypeptides of XMRV strains, where such fragments have an XMRV associated function or activity. An XMRV associated function or activity can be one or more of the functions or activities discussed below.

Assays for determining XMRV, or fragments thereof, functionality can be according to general methods known in the art (see e.g., Kurth 2010 Retroviruses: Molecular Biology, Genomics and Pathogenesis, Caister Academic Press, ISBN-10: 1904455557; Zhu 2010 Human Retrovirus Protocols: Virology and Molecular Biology (Methods in Molecular Biology), 1st Edition, Humana Press, ISBN-10: 1617375993).

Envelope Polypeptide Activity Assay

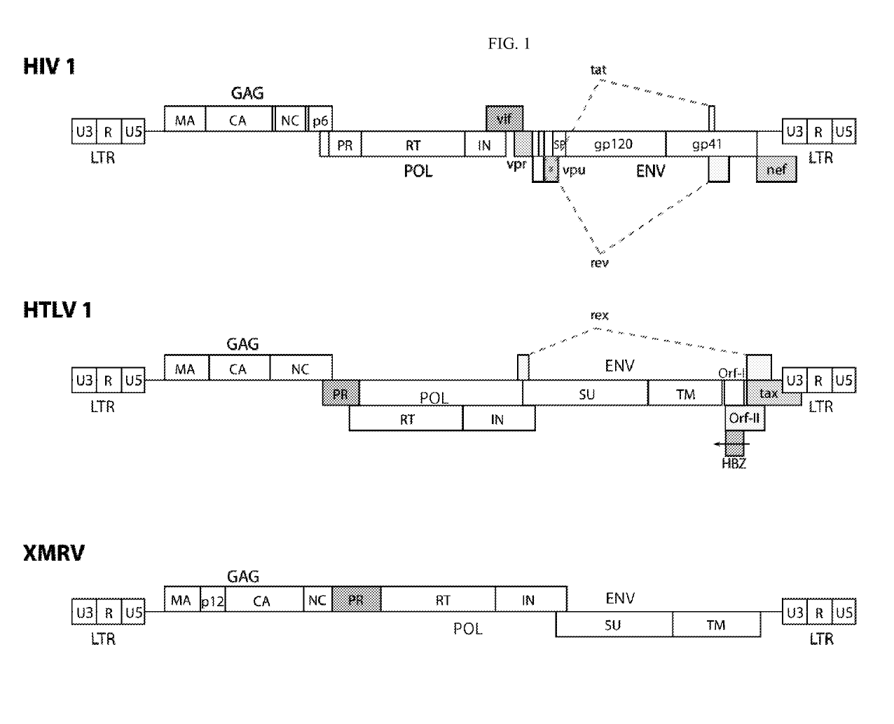

Envelope polypeptide is a transcribed polypeptide corresponding to the Env region of the XMRV genome (see FIG. 1). Envelope polypeptide of VP62 has a UniProt Accession number of Q27ID8 (SEQ ID NO: 160) and can be 645 amino acids in length Amino acid positions discussed below are according to UniProt Accession number of Q27ID8; one of ordinary skill can determine corresponding amino acid positions in an XMRV variant described herein.

A functional XMRV envelope polypeptide, a functional fragment thereof, or a functional component thereof (e.g., SU, TM, R-peptide), can have one or more of the following structural features or functions: an extracellular topological domain at amino acid positions 34-585; a helical transmembrane region at amino acid positions 586-606; a cytoplasmic topological domain at amino acid positions 607-640; a receptor-binding domain (RBD) at amino acid positions 32-237; a fusion peptide region at amino acid positions 447-467; an immunosuppression region at amino acid positions 513-529; a coiled coil region at amino acid positions 490-510; a CXXC motif at amino acid positions 311-314; a CX6CC motif at amino acid positions 530-538; a YXXL motif containing an endocytosis signal at amino acid positions 630-633; and a Pro-rich region at amino acid positions 234-283. A functional XMRV envelope polypeptide, a functional fragment thereof, or a functional component thereof (e.g., SU, TM, R-peptide), can have one or more of the following structural features or functions: a cleavage (by host) site at amino acid position 444-445; or a cleavage (by viral protease p14) site at amino acid position 624-625. Positions listed above can be relative positions where functionality is preserved, depending on the XMRV variant. A YXXL motif of the XMRV envelope protein is involved in determining the site of viral release at the surface of infected mononuclear cells and promotes endocytosis. The immunosuppressive region (e.g., a relatively conserved 17 amino acid region) can inhibit immune function.

[ 0 0 9 0 ] A functional XMRV gp70 envelope protein, a functional fragment thereof, or a functional component thereof (e.g., SU, TM, R-peptide), can have one or more of the structural features or functions discussed herein. The XMRV envelope glycoprotein is cleaved into three chains as follows: surface protein (SU) at amino acid position 34-444; transmembrane protein (TM) at amino acid position 445-645; and R-protein at amino acid positions 625-645. Specific enzymatic cleavages (e.g., in vivo) can yield mature XMRV proteins. Envelope glycoproteins are synthesized as an inactive precursor that is N-glycosylated and processed (e.g., by host cell furin or by a furin-like protease in the Golgi) to yield the mature SU and TM proteins. The cleavage site between SU and TM can require the minimal sequence [KR]-X-[KR]-R. The R-peptide is released from the C-terminus of the cytoplasmic tail of the TM protein upon particle formation as a result of proteolytic cleavage by the viral protease. Cleavage of the R-peptide can be required for TM to become fusogenic. The TM protein and the R-peptide is palmitoylated. The R-peptide is membrane-associated through its palmitate.

The mature envelope protein (Env) consists of a trimer of SU-TM heterodimers attached by a labile interchain disulfide bond. The activated Env consists of SU monomers and TM trimers. The SU protein is not anchored to the XMRV viral envelope, but associates with the XMRV virion surface through its binding to TM. Both SU and TM proteins may be concentrated at the site of budding and incorporated into an XMRV virion by contacts between the cytoplasmic tail of Env and the N-terminus of Gag. The surface protein (SU) attaches the XMRV virus to the host cell by binding to its receptor. This interaction activates a thiol in a CXXC motif of the C-terminal domain, where the other Cys residue participates in the formation of the intersubunit disulfide.

The CXXC motif is highly conserved across a broad range of retroviral envelope proteins, including XMRV envelope protein. The CXXC motif may participate in the formation of a labile disulfide bond (e.g., with the CX6CC motif present in the transmembrane protein). Isomerization of the intersubunit disulfide bond to an SU intrachain disulfide bond may occur upon receptor recognition in order to allow membrane fusion. The activated thiol can attack the disulfide and cause its isomerization into a disulfide isomer within the motif This can lead to SU displacement and TM refolding, and may activate its fusogenic potential by unmasking its fusion peptide. Fusion can occur at the host cell plasma membrane. The transmembrane protein (TM) can act as a class I viral fusion protein. The TM protein can have at least 3 conformational states: pre-fusion native state, pre-hairpin intermediate state, and post-fusion hairpin state. During XMRV viral and target cell membrane fusion, the coiled coil regions (heptad repeats) assume a trimer-of-hairpins structure, positioning the fusion peptide in close proximity to the C-terminal region of the ectodomain. The formation of this structure may drive apposition and subsequent fusion of viral and target cell membranes. Membranes fusion leads to delivery of the nucleocapsid into the cytoplasm.

The CC amino acid sequence comprised by AALKEECCFYADHT (SEQ ID NO: 6), amino acids 420-433 of the XMRV ENV polypeptide, is thought to interact with host kinases.

Gag-Pol Polypeptide Activity Assay

Gag-Pol polypeptide is a transcribed polypeptide corresponding to the Gag-Pol region of the XMRV genome (see FIG. 1). Gag-Pol polypeptide of VP62 has a UniProt Accession number of AlZ651 (SEQ ID NO: 161) and can be 1733 amino acids in length Amino acid positions discussed below are according to UniProt Accession number of AlZ651; one of ordinary skill can determine corresponding amino acid positions in an XMRV variant described herein.

A functional XMRV Gag-Pol polypeptide, a functional fragment thereof, or a functional component thereof (e.g., matrix protein p15; RNA-binding phosphoprotein p12; capsid protein p30; nucleocapsid protein p10; protease p14; reverse transcriptase/ribonuclease H; integrase p46) can have one or more of the following structural features or functions: a peptidase A2 domain at amino acid position 559-629; a reverse transcriptase domain at amino acid position 739-930; and RNase H domain at amino acid position 1172-1318; an integrase catalytic domain at amino acid position 1442-1600; a CCHC-type domain at amino acid position 500-517; a coiled coil at amino acid position 436-476; a PTAP/PSAP motif at amino acid position 109-112; a LYPX(n)L motif at amino acid position 128-132; a PPXY motif at amino acid position 161-164; a Pro-rich region at amino acid position 71-191; and Pro-rich region at amino acid position 71-168. A functional XMRV Gag-Pol polypeptide, a functional fragment thereof, or a functional component thereof (e.g., matrix protein p15; RNA-binding phosphoprotein p12; capsid protein p30; nucleocapsid protein p10; protease p14; reverse transcriptase/ribonuclease H; integrase p46) can have one or more of the following structural features or functions: a protease active site at amino acid position 564; a magnesium metal binding catalytic site for reverse transcriptase activity at amino acid positions 807, 881, or 882; a magnesium metal binding site for RNase H activity at amino acid positions 1181, 1219, 1240, or 1310; a magnesium metal binding catalytic site for integrase activity at amino acid positions 1453 or 1512; and a cleavage site by viral protease p14 at amino acid positions 129-130, 213-214, 476-477, 532-533, 657-658, or 1328-1329. Positions listed above can be relative positions where functionality is preserved, depending on the XMRV variant.

A functional XMRV Gag-Pol polypeptide, a functional fragment thereof, or a functional component thereof (e.g., matrix protein p15; RNA-binding phosphoprotein p12; capsid protein p30; nucleocapsid protein p10; protease p14; reverse transcriptase/ribonuclease H; integrase p46) can have one or more of the structural features or functions discussed herein. The Gag-Pol polyprotein can be translated as a gag-pol fusion protein by episodic readthrough of the gag protein termination codon. The Gag-Pol polyprotein can be cleaved into seven polypeptide chains, each described below. Gag-Pol polyprotein can play a role in budding and can be processed by the viral protease during virion maturation outside the cell. During budding, Gag-Pol polyprotein can recruit, in a PPXY-dependent or independent manner, Nedd4-like ubiquitin ligases that can conjugate ubiquitin molecules to Gag, or to Gag binding host factors. Interaction with HECT ubiquitin ligases may link the XMRV viral protein to the host ESCRT pathway and facilitate release. Specific enzymatic cleavages by the viral protease can yield mature proteins. The protease can be released by autocatalytic cleavage. The polyprotein can be cleaved during and after budding in process is termed maturation.

A functional p15 matrix protein (Ma/E), or a functional fragment or component thereof, can have one or more of the structural features or functions discussed herein. Matrix protein p15 can target Gag and gag-pol polyproteins to the plasma membrane via a multipartite membrane binding signal, that includes its myristoylated N-terminus Matrix protein p15 can also mediates nuclear localization of the preintegration complex. A p15 matrix protein can be located at amino acid position 2-129 of the Gag-Pol polypeptide. Such position can be relative where functionality is preserved, depending on the XMRV variant.

A functional p12 RNA-binding phosphoprotein, or a functional fragment or component thereof, can have one or more of the structural features or functions discussed herein. p12 RNA-binding phosphoprotein corresponds to nucleotide positions. RNA-binding phosphoprotein p12 is post-translationally phosphorylated on serine residues. A p12 RNA-binding phosphoprotein can be located at amino acid position 130-213 of the Gag-Pol polypeptide. Such position can be relative where functionality is preserved, depending on the XMRV variant.

A functional p30 capsid protein, or a functional fragment or component thereof, can have one or more of the structural features or functions discussed herein. Capsid protein p30 can form a spherical core of the XMRV virion that encapsulates the genomic RNA-nucleocapsid complex. Capsid protein p30 is a homohexamer, that further associates as homomultimer. The XMRV virus core is composed of a lattice formed from hexagonal rings, each containing six capsid monomers. Capsid protein p30 is post-translational sumoylated, which can be required for virus replication. A p30 capsid protein can be located at amino acid position 214-476 of the Gag-Pol polypeptide. Such position can be relative where functionality is preserved, depending on the XMRV variant.

A functional p10 nucleocapsid protein, or a functional fragment or component thereof, can have one or more of the structural features or functions discussed herein. Nucleocapsid protein p10 is involved in the packaging and encapsidation of two copies of the genome. Nucleocapsid protein p10 can bind with high affinity to conserved UCUG elements within the packaging signal, located near the 5′-end of the XMRV genome, where such binding can be dependent on genome dimerization. The nucleocapsid protein p10 released from Pol polyprotein (NC-pol) can be a few amino acids shorter than the nucleocapsid protein p10 released from Gag polyprotein (NC-gag). A p10 nucleocapsid protein can be located at amino acid position 477-532 of the Gag-Pol polypeptide. Such position can be relative where functionality is preserved, depending on the XMRV variant.

A functional p14 protease, or a functional fragment or component thereof, can have one or more of the structural features or functions discussed herein. Aspartyl protease (EC=3.4.23.-) can mediate proteolytic cleavages of Gag and Gag-Pol polyproteins during or shortly after the release of the virion from the plasma membrane. Cleavages can take place as an ordered, step-wise cascade to yield mature proteins, a process called maturation. Aspartyl protease can display maximal activity during the budding process just prior to particle release from the cell. The protease is a homodimer, whose active site consists of two apposed aspartic acid residues. A p14 protease can be located at amino acid position 533-657 of the Gag-Pol polypeptide. Such position can be relative where functionality is preserved, depending on the XMRV variant.

A functional p80 Reverse transcriptase/ribonuclease H, or a functional fragment or component thereof, can have one or more of the structural features or functions discussed herein. Reverse transcriptase/ribonuclease H (EC=2.7.7.49; EC=2.7.7.7; EC=3.1.26.4) (RT) is a multifunctional enzyme that can convert the viral dimeric XMRV RNA genome into dsDNA in the cytoplasm, shortly after virus entry into the cell. The reverse transcriptase is a monomer. Reverse transcriptase/ribonuclease H can display a DNA polymerase activity that can copy either DNA or RNA templates, and a ribonuclease H (RNase H) activity that can cleave the RNA strand of RNA-DNA heteroduplexes in a partially processive 3′ to 5′ endonucleasic mode. Conversion of viral genomic RNA into dsDNA can requires multiple steps, as follows. A tRNA can bind to the primer-binding site (PBS) situated at the 5′ end of the viral RNA. RT can use the 3′ end of the tRNA primer to perform a short round of RNA-dependent minus-strand DNA synthesis. The reading can proceed through the U5 region and can end after the repeated (R) region which is present at both ends of viral RNA. The portion of the RNA-DNA heteroduplex can be digested by the RNase H, resulting in a ssDNA product attached to the tRNA primer. This ssDNA/tRNA can hybridize with the identical R region situated at the 3′ end of viral RNA. This template exchange, known as minus-strand DNA strong stop transfer, can be either intra- or intermolecular. RT can use the 3′ end of this newly synthesized short ssDNA to perfom the RNA-dependent minus-strand DNA synthesis of the whole template. RNase H can digest the RNA template except for a polypurine tract (PPT) situated at the 5′ end of the XMRV genome. RNase H can proceed both in a polymerase-dependent (RNA cut into small fragments by the same RT performing DNA synthesis) and a polymerase-independent mode (cleavage of remaining RNA fragments by free RTs). Secondly, RT can perform DNA-directed plus-strand DNA synthesis using the PPT that has not been removed by RNase H as primers. PPT and tRNA primers can then removed by RNase H. The 3′ and 5′ ssDNA PBS regions can hybridize to form a circular dsDNA intermediate. Strand displacement synthesis by RT to the PBS and PPT ends can produce a blunt ended, linear dsDNA copy of the XMRV viral genome that includes long terminal repeats (LTRs) at both ends. The reverse transcriptase is an error-prone enzyme that lacks a proof-reading function. High mutations rate can be a direct consequence of this characteristic. RT can also display frequent template switching leading to high recombination rate. Recombination mostly occurs between homologous regions of the two copackaged RNA genomes. If these two RNA molecules derive from different viral strains (e.g., different XMRV strains), reverse transcription can give rise to highly recombinated proviral DNAs. A p80 Reverse transcriptase/ribonuclease H can be located at amino acid position 658-1328 of the Gag-Pol polypeptide. Such position can be relative where functionality is preserved, depending on the XMRV variant.

A functional p46 integrase, or a functional fragment or component thereof, can have one or more of the structural features or functions discussed herein. Integrase can catalyze viral DNA integration into a host chromosome, by performing a series of DNA cutting and joining reactions. Integrase activity can take place after XMRV virion entry into a cell and reverse transcription of the XMRV RNA genome in dsDNA. The first step in the integration process can be 3′ processing. This step can require a complex comprising the XMRV viral genome, matrix protein and integrase (i.e., a pre-integration complex (PIC)). The integrase protein can remove 2 nucleotides from each 3′ end of the XMRV viral DNA, leaving recessed CA OH's at the 3′ ends. In the second step that can require cell division, the PIC enters cell nucleus. In the third step, termed strand transfer, the integrase protein can join the previously processed 3′ ends to the 5′ ends of strands of target cellular DNA at the site of integration. The fourth step can be XMRV viral DNA integration into a host chromosome. A p46 integrase can be located at amino acid position 1329-1733 of the Gag-Pol polypeptide. Such position can be relative where functionality is preserved, depending on the XMRV variant.

Gammaretrovirus Core Encapsidation Signal

A functional XMRV, or a functional fragment or component thereof, can have a structurally or functionally active gammaretrovirus core encapsidation signal. Gammaretrovirus core encapsidation signal is an RNA element known to be essential for stable dimerization and efficient genome packaging during virus assembly. Dimerisation of the viral RNA genomes can act as an RNA conformational switch that exposes conserved UCUG elements and enables efficient genome encapsidation. A functional RNA gammaretrovirus core encapsidation signal has a structure composed of three stem-loops, two of which, SL-C and SL-D, form a single co-axial extend helix. A substitution of an XMRV nucleic acid sequence may have an effect on the functionality of the gammaretrovirus core encapsidation signal.

XMRV Virion Assay

Function of XMRV, or a functional fragment or component thereof, can be according to an assay that determines the number of XMRV virion particles produced in a subject or sample. Analysis of the number of XMRV virion particles as a means of assessing XMRV function can be according to electron micrographic analysis. XMRV virion particles can be from direct isolation from a subject, from cultured primary cells, or from co-cultured indicator cells (e.g., LNCaP cells).

Immune Response to XMRV

Function of XMRV, or a functional fragment or component thereof, can be according to an immune response generated in a subject in vivo (see e.g., Lombardi et al. 2011 In Vivo 25(2)). For example, a functional XMRV, or functional fragment or component thereof, can effect a cytokine or chemokine signature in a subject as described in Lombardi et al. 2011.

Function of XMRV, or a functional fragment or component thereof, can be according to a humoral response in a subject that produces anti-XMRV antibodies. Detection of anti-XMRV antibodies can be according to discussion herein.

Ex Vivo Fitness

Function of XMRV, or a functional fragment or component thereof, can be according to a measure of ex vivo fitness through a growth competition assay. For example, two or more XMRV strains (or an XMRV and a control) can be compared with respect to ex vivo fitness by exposing a cell culture to both XMRV and subsequently assessing which strain exhibits a higher growth rate or viral titer. As another example, two or more XMRV strains (or an XMRV and a control) can be compared with respect to ex vivo fitness by exposing a first cell culture to a first XMRV strain and a second cell culture to a second XMRV strain or a control and subsequently assessing which strain (or control) exhibits a higher growth rate or viral titer. It is understood that more than two XMRV strains can be assessed simultaneously or concurrently.

Viral Infectiousness Assay

Function of XMRV, or a functional fragment or component thereof, can be according to an assay that determines the ability of an XMRV to infect a cell (e.g., in vitro tissue culture) or a subject (e.g., an animal model for viral infectivity). For example, a functional XMRV, or a functional fragment or component thereof, can be an XMRV that can infect a cell in culture according to a modified Derse assay, which measures infectious viral particles (see e.g., KyeongEun , 18th Conference of Retrovirus and Opportunistic Infections, Session 43, Paper #215, Development of a GFP-indicator Cell Line for the Detection of XMRV).

Reverse Transcriptase Activity

Function of XMRV, or a functional fragment or component thereof, can be according to a reverse transcriptase activity assay. For example, reverse transcriptase activity can be detected in a viral suspension prepared from a cell culture exposed to an XMRV. Assaying reverse transcriptase activity can be according to methods know in the art (e.g., Colorimetric Reverse Transcriptase Immunoassay, Roche Applied Science; Chemiluminescence Reverse Transcriptase Assay, Promega).

Transformation Ability Assay

Function of XMRV, or a functional fragment or component thereof, can be according to an assay that determines the ability of XMRV infection to immortalize or modify a phenotype of primary cell or cell culture. For example, a change in cluster of differentiation (CD) or cell receptors on a cell surface can be monitored or determined so as to characterize transformation ability of an XMRV.

Cell Death

Function of XMRV, or a functional fragment or component thereof, can be according to an assay that determines susceptibility of cells (e.g., cells of a subject, sample, or a cell line) to cell syncytia or cell death. Analysis of the response of cells to exposure or infection to XMRV, including cell syncytia or cell death, as a means of assessing XMRV function can be according to electron micrographic analysis. Analysis of cell syncytia can be from direct isolation from a subject, from cultured primary cells, or from co-cultured indicator cells (e.g., LNCaP cells).

Plaque Assays

Function of XMRV, or a functional fragment or component thereof, can be according to an assay that determines plaque assays formed in cell culture (e.g., agar suspended cell culture; adherent cell culture) as a result of XMRV infection.

TCID₅₀

Function of XMRV, or a functional fragment or component thereof, can be according to an assay that determines tissue culture infective dose (TCID₅₀). Tissue culture infective dose is the quantity of cytopathic agent (e.g., XMRV titer) that will produce cell death in fifty percent of cell cultures inoculated.

Molecular Engineering

Design, generation, and testing of the variant nucleotides, and their encoded polypeptides, having the above required percent identities and retaining a required function or activity is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide or polypeptide variants having, for example, at least 95-99% identity to the reference sequence described herein and screen such for desired phenotypes according to methods routine in the art. Generally, conservative substitutions can be made at any position so long as the required activity is retained.

Nucleotide or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tm=81.5° C.+16.6(log 10[Na+])+0.41(fraction G/C content)−0.63(% formamide)−(600/1). Furthermore, the Tm of a DNA:DNA hybrid is decreased by 1-1.5° C. for every 1% decrease in nucleotide identity (see e.g., Sambrook and Russel, 2006).

Nucleic acids can be inserted into host cells for a variety of reasons. Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides, protein aptamers, nucelotide aptamers, and RNA interference (RNAi) (e.g., small interfering RNAs (siRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, Tex.; Sigma Aldrich, Mo.; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinofrmatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Methods of Detecting XMRV Strains

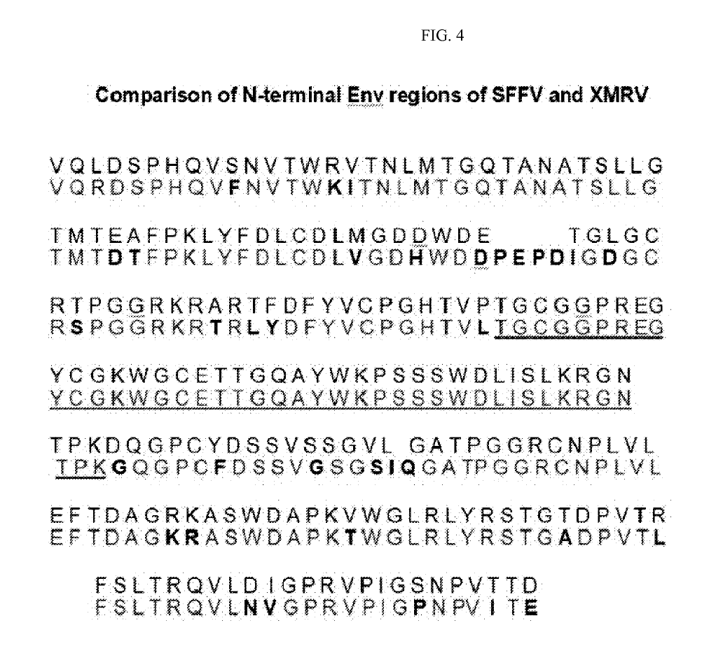

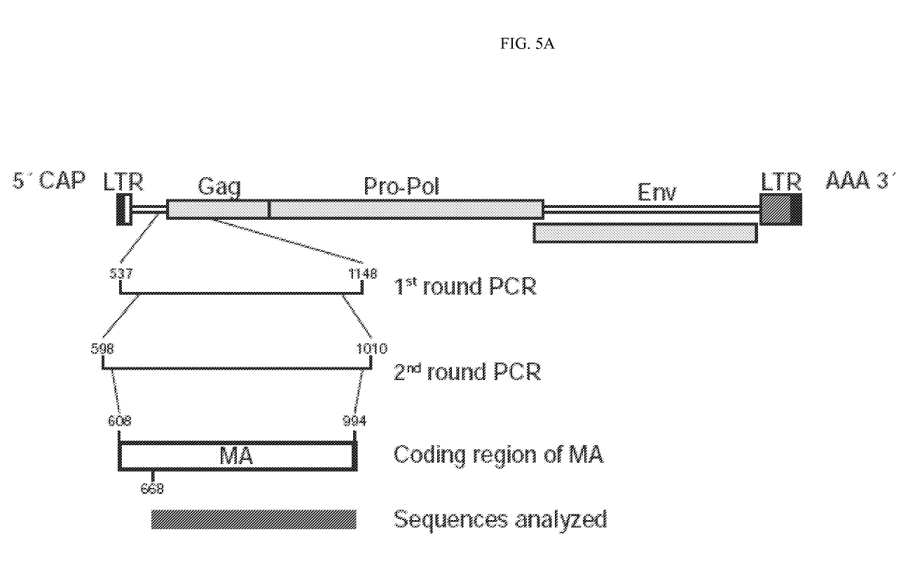

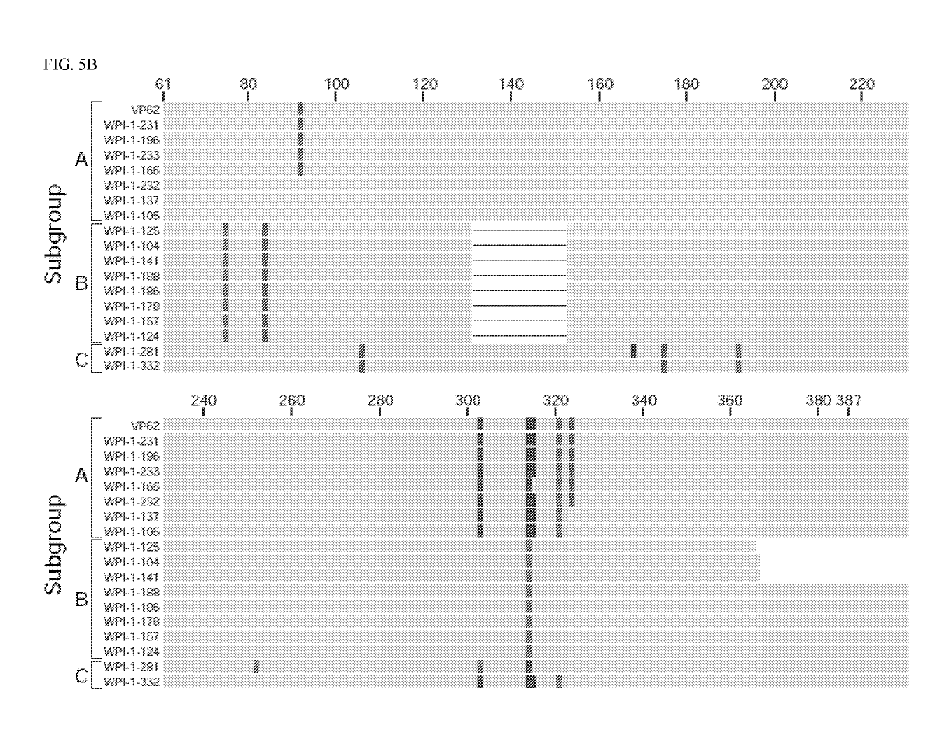

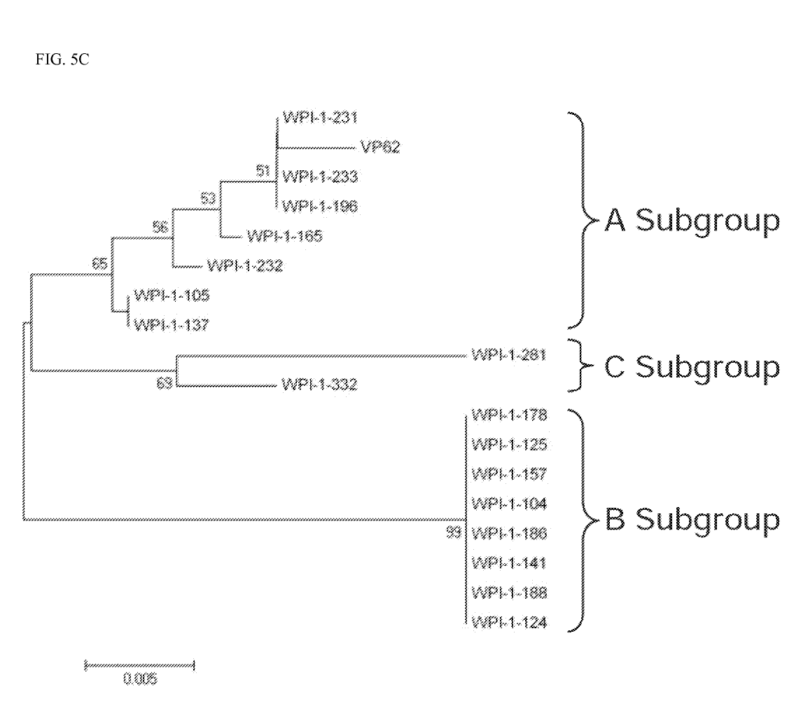

One aspect of the present disclosure provides methods of identifying polynucleotides or polypeptides characteristic of an XMRV strain or group thereof. For example, analysis of nucleotide or amino acid positions/residues that vary between XMRV strains can allow detection of identification of such strains. As another example, methods described herein can be used to detect and distinguish between various groupings of XMRV strains, such as P- and X-XMRV isolates. For example, FIG. 4 shows a comparison of the N-terminal regions of the Env protein of XMRV and SFFV according to the methods described herein. This type of amino acid comparison can be used to assign a sequence from a clinically isolated XMRV as a particular XMRV strain, or group thereof, such as P- or X-XMRV.

Methods described herein can provide for identification of nucleotide or amino acid variation in an XMRV strain. In some cases, variation in XMRV sequence can be clinically relevant, and lead to variation in XMRV pathogenicity, immune response, or disease response. Such variation can be in one or more XMRV polynucleotide sequence, variation in one or more XMRV polypeptide sequence, or variation in one or more of both XMRV polynucleotide and XMRV polypeptide sequences.

Retrovirus detection methods are generally known in the art and, provided with sequence information herein, can be adapted for detection of XMRV strains.

XMRV can be detected by detecting antibodies to XMRV in a subject. To detect anti-XMRV antibodies, a cell line expressing SFFV Env proteins can be incubated with plasma of a subject. The cell line can then be subjected to methods of determining whether an antibody from the subject bound to the SFFV Env protein, such as by flow cytometry. Detecting anti-XMRV antibodies can be done by subjecting subject plasma to ELISA and identifying antibodies. Methods for the detection of XMRV by detecting antibodies are described in PCT/US2010/039208, U.S. patent application Ser. No. 12/818,880 and U.S. patent application Ser. No. 12/818,893, each of which is incorporated herein by reference in its entirety.

XMRV can be detected by detecting XMRV proteins. XMRV proteins can be detected by running a sample suspected of comprising XMRV on an SDS-PAGE gel, performing a Western blot, and detecting XMRV proteins on the blot. Proteins which can be detected include gag or env proteins. Antibodies that can be used to detect XMRV proteins include antibodies against SFFV, and specifically can include the antibody known as 7C10. XMRV proteins can be detected using polyclonal sera against X-MLV (NZB); polyclonal sera aganst E-MLV (R-MLV), SU (gp70), p30 (CA) and p10 (NC); or a monoclonal antibody against MLV p30 (CA). Methods for the detection of XMRV by detecting XMRV proteins are described in PCT/US2010/039208, U.S. patent application Ser. No. 12/818,880 and U.S. patent application Ser. No. 12/818,893, each of which is incorporated herein by reference in its entirety.

XMRV can be detected by detecting proviral polynucleic acids in an infected cell. Detecting proviral polynucleic acids can comprise performing PCR to amplify and visualize or sequence the DNA. Detecting proviral polynucleic acids can comprise performing RT-PCR to amplify and visualize or sequence virion RNA. The PCR or RT-PCR can be conventional PCR or RT-PCR, or can comprise additional amplification, purification or cycling steps. Methods for the detection of XMRV by detecting proviral polynucleic acids are described in PCT/US2010/039208, U.S. patent application Ser. No. 12/818,880 and U.S. patent application Ser. No. 12/818,893, each of which is incorporated herein by reference in its entirety.

XMRV can be detected by infection of cultured or co-cultured cells. To detect XMRV by infecting cultured cells, cell-free samples suspected of comprising XMRV can be exposed to cultured Derse or LNCaP cells, and the infection status of the Derse or LNCaP cells can be monitored. To detect XMRV by infecting co-cultured cells, cells suspected of comprising XMRV, including plasma or activated peripheral blood mononuclear cells (PBMCs), can be co-cultured with Derse cells or LNCaP cells, and then the XMRV status of the Derse or LNCaP cells can be determined Methods for the detection of XMRV by the infection of co-cultured cells are described in PCT/US2010/039208, U.S. patent application Ser. No. 12/818,880 and U.S. patent application Ser. No. 12/818,893, each of which is incorporated herein by reference in its entirety.

XMRV can be detected by direct isolation of XMRV proteins from plasma of subjects by immunoprecipitation of XMRV with antibodies, followed by detection of the proteins by a method described herein. For example, the antibody used for immunoprecipitation of XMRV can be anti-X-MLV (BALB-V2). The proteins can be run on an SDS-PAGE gel, Western blotted, and the blot probed with anti-R-MuLV Gag antibodies.

The foregoing methods, and other methods described herein, can be used to generally detect or discriminate between various strains or XMRV, or groups thereof, such as X-XMRV or P-XMRV.

Identifying Particular XMRV Strains or Groups.

In some aspects, a method of identifying polynucleotides or polypeptides particular to an XMRV strain, or group thereof, such as P- or X-XMRV, can comprise obtaining, amplifying and sequencing viral polynucleotides or polypeptides. For example, based on disclosure of sequences described herein, one of ordinary skill can sequence nucleic acids present in a sample and directly determine whether and what type of XMRV strain, or group thereof such as X-XMRV or P-XMRV, are present, or if more than one are present, distinguish there between.

Similarly, direct sequencing of polypeptides, either present in a sample or translated from a nucleic acid, can directly determine whether X-XMRV or P-XMRV associated proteins are present, or if both are present, distinguish there between. Such methods include, but are not limited to protein (peptide) sequencing (see e.g., Steen and Mann, Nature Reviews Mol. Cell Biol. 5:699, 2004).

Based on disclosure of sequences described herein, one of ordinary skill can design primers specific for an XMRV strain, or a group thereof, such as X-XMRV, P-XMRV, or X-XMRV and P-XMRV, where, for example, such primers can be used to detect one of X-XMRV or P-XMRV, or distinguish between X-XMRV and P-XMRV. Primers can be designed for any region of XMRV that contains a difference in nucleic acid sequence between two or more XMRV strains, or groups such as X-XMRV or P-XMRV. For example, primers can be designed for one of more of an envelope or gag region of XMRV.

For example, primer(s) specific for an XMRV strain, or a group thereof, such as X-XMRV or P-XMRV, can be used, where detection can be based on presence or absence of an amplification product (e.g., presence or absence of a band on gel electrophoresis).

As another example, primer(s) specific for an XMRV strain, or a group thereof, such as X-XMRV or P-XMRV, can be used, where detection can be based on an amplification product size (e.g., band size on gel electrophoresis).

In some embodiments, the primers used to amplify the viral polynucleotides can be primers designed to amplify Env-encoding polynucleotides. Such primers can comprise P5588F (5′-GTGTGGGTACGCCGGCACCAGAC-3′, SEQ ID NO:2) and P6304R (5′-TGCATCGACCCCCCGGTGTGGC-3′, SEQ ID NO:3). In some embodiments, the polynucleotide amplification can comprise two rounds of PCR, wherein the primers for the second round amplify Env-encoding polynucleotides, and comprise P5641F (5′-CTACACCGTCCTGCTGACAACC-3′, SEQ ID NO:4) and P6171R (5′-TGCCTGTCCAGTGGTCTCACATC-3′, SEQ ID NO:5).

Variation between polypeptide sequences can be identified through the use of antibodies that are specific for a particular amino acid motif which is present in a first, but not in a second, polypeptide sequence. Based on disclosure of sequences described herein, one of ordinary skill can generate antibodies useful for detection of XMRV strains, or a group thereof, such as X-XMRV or P-XMRV, or distinguishing there between. Antibodies can be generated to be specific for any region of XMRV that contains a difference in amino acid sequence between XMRV strains, or groups thereof, such as X-XMRV or P-XMRV. For example, antibodies can be designed for one of more of an envelope or gag region of XMRV, or the XMRV virion.

Capture epitopes can be designed that specifically recognize one of an anti-XMRV strain antibody, or a group thereof, such as an anti-X-XMRV antibody or an anti-P-XMRV antibody, in a subject or a sample from the subject. For example, antibodies in a subject can be detected according to a standard protocol, such as ELISA

Antibodies specific for an XMRV strain, or a group thereof, such as X-XMRV or P-XMRV, (see Table 1, e.g., 20 amino acid insert of P-XMRV) can be directly detected in a sample (e.g., a sample from a subject), where presence of such antibodies indicates a humoral immune response to the XMRV strain or group thereof, such as X-XMRV or P-XMRV.

Antibodies can be developed with specific affinity for an XMRV strain associated proteins, or a proteins associated with group thereof, such as X-XMRV or P-XMRV. Such antibodies specific for associated proteins can be used in an antibody-based assay for direct detection of XMRV virions or proteins in a sample (e.g., a sample from a subject).

One aspect provides distinguishing an XMRV strain described herein, for example on the basis of a polynucleotide or polypeptide described herein, from another XMRV virus, such as VP62 (SEQ ID NO: 1, SEQ ID NO: 162), VP35 (SEQ ID NO: 163), or VP42 (SEQ ID NO: 164). For example, detection of any of the amino acid changes or nucleic acid changes described herein not possessed by VP62 (SEQ ID NO: 1, SEQ ID NO: 162), VP35 (SEQ ID NO: 163), or VP42 (SEQ ID NO: 164) can be a determination that the detected XMRV is not VP62 (SEQ ID NO: 1, SEQ ID NO: 162), VP35 (SEQ ID NO: 163), or VP42 (SEQ ID NO: 164), respectively.

Sample and Subject

Methods for the detection or identification of clinically relevant polynucleotides or polypeptides of an XMRV strain described herein are generally performed on a subject or on a sample from a subject. Subject can be infected or suspected of being infected with XMRV. A sample can contain or be suspected of containing XMRV. A sample can be a biological sample from a subject.

The subject can be a subject having, diagnosed with, suspected of having, or at risk for developing a disease or disorder associated with XMRV. An XMRV-associated disease or disorder includes, but is not limited to, prostate cancer (e.g., prostate cancer tumors homozygous for a R462Q mutation), CFS, autism and autism spectrum disorders, gulf war syndrome (GWS), multiple sclerosis (MS), Amyotrophic Lateral Sclerosis (ALS), Parkinson's disease, Niemann-Pick Type C Disease, fibromyalgia, autism, chronic Lyme disease, non-epileptic seizures, Thymoma, myelodysplasia, Immune Thrombocytopenic Purpura (IPT), Mantle Cell Lymphoma (MCL), and Chronic Lymphocytic Leukemia lymphoma (CLL).

An XMRV-associated disease or disorder includes, but is not limited to an XMRV-related lymphoma or an XMRV-related neuroimmune disease. Examples of an XMRV-related lymphoma include, but are not limited to an XMRV-related Mantle Cell Lymphoma (MCL) and a Chronic Lymphocytic Leukemia lymphoma (CLL). Examples of an XMRV-related neuroimmune disease include, but are not limited to Chronic Fatigue Syndrome (CFS), fibromyalgia, Multiple Sclerosis (MS), Parkinson's Disease, Amyotrophic Lateral Sclerosis (ALS), autism spectrum disorder (ASD), and chronic lyme disease.

For example, a subject can be tested for the presence of an XMRV where the subject exhibits signs or symptoms of a disease or disorder associated with XMRV, such as a neuroimmune disease or a lymphoma. As another example, a subject can have been diagnosed with a disease or disorder associated with XMRV, such as a neuroimmune disease or a lymphoma. A subject can be considered at risk of developing a disease or disorder associated with XMRV, such as a neuroimmune disease or a lymphoma, includes, without limitation, an individual with a familial history of such disease or disorder, or an individual residing in a region comprising a cluster of individuals with such disease or disorder.

A determination of the need for detecting, diagnosing, monitoring, or managing an XMRV-related disease or disorder, such as a neuroimmune disease or a lymphoma, will typically be assessed by a history and physical exam consistent with the disease or condition at issue. Such assessment is within the skill of the art. The subject can be an animal subject, preferably a mammal, more preferably horses, cows, dogs, cats, sheep, pigs, mice, rats, monkeys, guinea pigs, and chickens, and most preferably a human.

For example, a subject can be one which fulfills the 1994 CDC Fukuda Criteria for CFS (Fukuda et al., Ann Intern Med 1994;121: 953-9); the 2003 Canadian Consensus Criteria (CCC) for ME/CFS (Carruthers et al, J Chronic Fatigue Syndrome 2003; 11:1-12; Jason et al., J Chronic Fatigue S 2004; 12:37-52), or both the Fukuda and CCC criteria. The CCC requires post-exertional malaise, which many clinicians believe is the sine qua non of ME/CFS. In contrast, the Fukuda and 1991 Oxford Criteria do not require exercise intolerance for a diagnosis of ME/CFS. The CCC further requires that subjects exhibit post-exertional fatigue, unrefreshing sleep, neurological/cognitive manifestations and pain, rather than these being optional symptoms.

As another example, the subject can be an animal, such as a laboratory animal that can serve as a model system for investigating a neuroimmune disease or lymphoma (see e.g., Chen, R. et al., Neurochemical Research 33: 1759-1767, 2008; Kumar, A., et al., Fundam. Clin. Pharmacol. Epub ahead of print, Jan. 10, 2009; Gupta, A., et al., Immunobiology 214: 33-39, 2009; Singh, A., et al., Indian J. Exp. Biol. 40: 1240-1244, 2002; Ford, R. J., et al. Blood 109: 4899-4906, 2007; Smith, M. R., et al., Leukemia 20: 891-893, 2006; Bryant, J., et al., Lab. Invest. 80: 557-573, 2000; M'kacher, R., et al., Cancer Genet Cytogenet. 143: 32-38, 2003).

A sample can be a blood sample, a serum sample, a plasma sample, a cerebrospinal fluid sample, or a solid tissue sample. For example, the sample can be a blood sample, such as a peripheral blood sample. As another example, a sample can be a solid tissue sample, such as a prostate tissue sample.

A sample can include cells of a subject. For example, a sample can include cells such as fibroblasts, endothelial cells, peripheral blood mononuclear cells, haematopoietic cells, or a combination thereof

Correlation of Presence of an XMRV Strain to Disease

Provided herein are methods for detecting, diagnosing, monitoring, or managing an XMRV-related disease or condition, for example, a. neuroimmune disease, an XMRV-related lymphoma, or both.

Detected presence or identification of an XMRV strain described herein in a subject, or a sample therefrom, can be correlated to a disease or condition associated with XMRV. For example, XMRV has been found at high prevalence in subjects diagnosed with CFS (Lombardi et al., 2009) and in certain types of prostate cancer. However, the present inventors postulate that XMRV can be a causal factor in many neurological and neuroimmune diseases, including but not limited to autism and autism spectrum disorders, gulf war syndrome (GWS), Amyotrophic Lateral Sclerosis (ALS), Niemann-Pick Type C Disease, fibromyalgia, autism, chronic Lyme disease, Gulf War Syndrome, and non-epileptic seizures; and that different disease diagnoses or symptoms are caused by various XMRV strains described herein.

Examples of an XMRV-related lymphoma include, but are not limited to an XMRV-related Mantle Cell Lymphoma (MCL) and a Chronic Lymphocytic Leukemia lymphoma (CLL). Examples of an XMRV-related neuroimmune disease include, but are not limited to Chronic Fatigue Syndrome (CFS), fibromyalgia, Multiple Sclerosis (MS), Parkinson's Disease, Amyotrophic Lateral Sclerosis (ALS), autism spectrum disorder (ASD), and chronic lyme disease. For example, CFS can be treated in a subject by administering a therapeutically effective amount of an anti-retroviral compound. As another example, MS, such as Atypical Multiple Sclerosis, can be treated in a subject by administering a therapeutically effective amount of an anti-retroviral compound or pharmaceutical composition including an anti-retroviral compound

In some cases, subjects infected with XMRV exhibit no persistent symptoms; i.e., they are apparently healthy. In other cases, subjects infected with XMRV are diagnosed with CFS. In other cases, subjects infected with XMRV are diagnosed with one or more cancer. In other cases, subjects infected with XMRV exhibit altered immune responses. In some cases, subjects infected with XMRV exhibit digestive-tract symptoms. Some subjects infected with XMRV develop multiple clinical symptoms, for example both CFS and cancer.

Therapeutic Methods

Also provided is a process of treating infection by an XMRV strain disclosed herein in a subject. Treating an XMRV infection can comprise administration of a therapeutically effective amount of an anti-retroviral agent, so as to suppress or prevent XMRV replication. Treating an infection by an XMRV strain disclosed herein can comprise administration of a therapeutically effective amount of a cocktail of anti-retroviral agents, so as to suppress or prevent XMRV replication.

Methods described herein are generally performed on a subject in need thereof. A subject can be according to discussion above. A subject in need of the therapeutic methods described herein can be diagnosed with an XMRV infection, or at risk thereof. A determination of the need for treatment will typically be assessed by a history and physical exam consistent with the disease or condition at issue. Diagnosis of the various conditions treatable by the methods described herein is within the skill of the art. The subject can be an animal subject, preferably a mammal, more preferably horses, cows, dogs, cats, sheep, pigs, mice, rats, monkeys, guinea pigs, and chickens, and most preferably a human.

An effective amount of an anti-retroviral agent described herein is generally that which can suppress or prevent XMRV replication. An effective amount of a cocktail of anti-retroviral agents described herein is generally that which can suppress or prevent XMRV replication. Alternatively, an effective amount of an anti-retroviral agent, or of a cocktail of anti-retroviral agents, is that which can suppress symptoms related to XMRV infection. Symptoms related to XMRV infection can be CFS symptoms, or they can be altered immune profiles as described herein.

Examples of anti-retroviral agents that can be used to manage or treat an XMRV-related neuroimmune disease or an XMRV-related lymphoma include, but are not limited to, acyclovir, penciclovir (famciclovir), gancyclovir (ganciclovir), deoxyguanosine, foscarnet, idoxuridine, trifluorothymidine, vidarabine, sorivudine, zidovudine (AZT, ZVD, azidothyidine, e.g., Retrovir), didanosine (ddl, e.g., Videx and Videx EC), zalcitabine (ddC, dideoxycytidine, e.g., Hivid), lamivudine (3TC, e.g., Epivir), stavudine (d4T, e.g., Zerit and Zerit XR), abacavir (ABC, e.g., Ziagen), emtricitabine (FTC, e.g., Emtriva (formerly Coviracil)), entecavir (INN, e.g., Baraclude), apricitabine (ATC), tenofovir (tenofovir disoproxil fumarate, e.g., Viread), adefovir (bis-POM PMPA, e.g., Preveon and Hepsera), multinucleoside resistance A, multinucleoside resistance B, nevirapine (e.g., Viramune), delavirdine (e.g., Rescriptor), efavirenz (e.g., Sustiva and Stocrin), etravirine (e.g., Intelence), adefovir dipivoxil, indinavir, ritonavir (e.g., Norvir), saquinavir (e.g., Fortovase, Invirase), nelfinavir (e.g., Viracept), agenerase, lopinavir (e.g., Kaletra), atasanavir (e.g., Reyataz), fosamprenavir (e.g., Lexiva, Telzir), tipranavir (e.g., Aptivus), darunavir (e.g., Prezista), amprenavir, deoxycytosine triphosphate, lamivudine triphosphate, emticitabine triphosphate, adefovir diphosphate, penciclovir triphosphate, lobucavir triphosphate, amantadine, rimantadine, zanamivir and oseltamivir, raltegravir (e.g., Isentress), elvitegravir (e.g., GS 9137 or JTK-303), MK-2048, maraviroc (e.g., Celsentri), enfuvirtide (e.g., Fuzeon), TNX-355, PRO 140, BMS-488043, plerixafor, epigallocatechin gallate, vicriviroc, aplaviroc, b12 (an antibody against HIV found in some long-term nonprogressors), griffithsin, DCM205, bevirimat, and vivecon. For example, one or more of AZT and cidofovir can be used to manage or treat an XMRV-related neuroimmune disease or an XMRV-related lymphoma. As another example, an interferon (e.g., interferon-β) can be used to manage or treat an XMRV-related neuroimmune disease or an XMRV-related lymphoma.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of an anti-retroviral agent, or a cocktail of anti-retroviral agents, can be employed in pure form or, where such forms exist, in pharmaceutically acceptable salt form and with or without a pharmaceutically acceptable excipient. For example, the compounds of the invention can be administered, at a reasonable benefit/risk ratio applicable to any medical treatment, in a sufficient amount to suppress or prevent XMRV replication, or to suppress symptoms related to XMRV infection.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where large therapeutic indices are preferred.

The specific therapeutically effective dose level for any particular subject will depend upon a variety of factors including the disorder being treated and the severity of the disorder; activity of the specific compound employed; the specific composition employed; the age, body weight, general health, sex and diet of the patient; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present invention will be decided by an attending physician within the scope of sound medical judgment.

Administration of an anti-retroviral agent, or a cocktail of anti-retroviral agents, can occur as a single event or over a time course of treatment. For example, an anti-retroviral agent, or a cocktail of anti-retroviral agents, can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for any XMRV-associated disease or condition described herein, such as an XMRV-related neuroimmune disease or an XMRV-related lymphoma.

An anti-retroviral agent, or a cocktail of anti-retroviral agents, can be administered simultaneously or sequentially with another agent, such as an antibiotic, an antiinflammatory, or another agent. For example, an anti-retroviral agent, or a cocktail of anti-retroviral agents, can be administered simultaneously with another agent, such as an antibiotic or an antiinflammatory. Simultaneous administration can occur through administration of separate compositions, each containing one or more of an anti-retroviral agent, or a cocktail of anti-retroviral agents, an antibiotic, an antiinflammatory, or another agent. Simultaneous administration can occur through administration of one composition containing two or more of an anti-retroviral agent, or a cocktail of anti-retroviral agents, an antibiotic, an antiinflammatory, or another agent. An anti-retroviral agent, or a cocktail of anti-retroviral agents, can be administered sequentially with an antibiotic, an antiinflammatory, or another agent. For example, an anti-retroviral agent, or a cocktail of anti-retroviral agents, can be administered before or after administration of an antibiotic, an antiinflammatory, or another agent.

Administration

Compositions described herein can be administered in a variety of means known to the art. The agents can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Compositions comprising an agent described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents will be known to the skilled artisan and are within the scope of the invention.

Delivery systems may include, for example, an infusion pump which may be used to administer the agent in a manner similar to that used for delivering insulin or chemotherapy to specific organs or tumors. Typically, using such a system, the agent(s) is administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart ploymeric carriers, and liposomes (see generally, Uchegbu and Schatzlein, eds. (2006) Polymers in Drug Delivery, CRC, ISBN-10: 0849325331). Carrier-based systems for biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Kits

Also provided are kits. Such kits can include the compositions of the present invention and, in certain embodiments, instructions for use. Such kits can facilitate performance of the methods described herein. When supplied as a kit, the different components of the composition can be packaged in separate containers and admixed immediately before use. Components include, but are not limited to probes, antigens, primers, reaction mixture components, anti-retroviral agents, etc., useful for detecting or identifying an XMRV strain described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Definitions and methods described herein are provided to better define the present invention and to guide those of ordinary skill in the art in the practice of the present invention. Unless otherwise noted, terms are to be understood according to conventional usage by those of ordinary skill in the relevant art.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

The methods utilized herein are well known to those of skill in the art. For instance, methods related to detecting XMRV infections can be as in U.S. patent application Ser. Nos. 12/818,880 and 12/818,893, each of which is incorporated herein by reference in its entirety.

### Example 1

This example describes methods that can be used to obtain nucleic acid samples from subjects.

DNA and RNA isolation. Whole blood can be drawn from subjects by venipuncture using standardized phlebotomy procedures into 8-mL greencapped Vacutainers containing the anti-coagulant sodium heparin (Becton Dickinson). Plasma can be collected by centrifugation, aspirated and stored at −80° C. for later use. The plasma can be replaced with PBS and the blood resuspended and further diluted with an equal volume of PBS. PBMCs can be isolated by layering the diluted blood onto Ficoll-Paque PLUS (GE Healthcare), centrifuging for 22 min at 800 g, aspirating the PBMC layer and washing it once in PBS. The PBMCs (approximately 2×10⁷ cells) can be centrifuged at 500 g for 7 min and either stored as frozen unactivated cells in 90% FBS and 10% DMSO at −80° C. for further culture and analysis or resuspended in TRIzol (Invitrogen) and stored at −80° C. for DNA and RNA extraction and analysis. DNA can be isolated from TRIzol according the to manufacturer's protocol and also can be isolated from frozen PBMC pellets using the QIAamp DNA Mini purification kit (QIAGEN) according to the manufacturer's protocol and the final DNA can be resuspended in RNase/DNase free water and quantified using the Quant-iT™ Pico Green dsDNA Kit (Invitrogen). RNA can be isolated from TRIzol according to the manufacturer's protocol and quantified using the Quant-iT Ribo Green RNA kit (Invitrogen). cDNA can be made from RNA using the iScript Select cDNA synthesis kit (Bio-Rad) according to the manufacturer's protocol.

### Example 2

This example describes methods of amplifying, and determining the nucleic acid sequence of, XMRV polynucleotides.

PCR. Nested PCR can be performed with separate reagents in a separate laboratory room designated to be free of high copy amplicon or plasmid DNA. Negative controls in the absence of added DNA can be included in every experiment. Identification of XMRV gag and env genes can be performed by PCR in separate reactions. Reactions can be performed as follows: 100 to 250 ng DNA, 2 μL of 25 mM MgCl2, 25 μL of HotStart-IT FideliTaq Master Mix (USB Corporation), 0.75 μL of each of 20 μM forward and reverse oligonucleotide primers in reaction volumes of 50 μL. For identification of gag, 419F (5′-ATCAGTTAACCTACCCGAGTCGGAC-3′) (SEQ ID NO: 7) and 1154R (5′-GCCGCCTCTTCTTCATTGTTCTC-3′) (SEQ ID NO: 8) can be used as forward and reverse primers. For env, 5922F (5′-GCTAATGCTACCTCCCTCCTGG-3′) (SEQ ID NO: 9) and 6273R (5′-GGAGCCCACTGAGGAATCAAAACAGG-3′) (SEQ ID NO: 10) can be used. For both gag and env PCR, 94° C. for 4 min initial denaturation can be performed for every reaction followed by 94° C. for 30 seconds, 57° C. for 30 seconds and 72° C. for 1 minute The cycle can be repeated 45 times followed by final extension at 72° C. for 2 minutes. Six microliters of each reaction product can be loaded onto 2% agarose gels in TBE buffer with 1 kb+ DNA ladder (Invitrogen) as markers. PCR products can be purified using Wizard SV Gel and PCR Clean-Up kit (Promega) and sequenced. PCR amplification for sequencing full-length XMRV genomes can be performed on DNA amplified by nested or semi-nested PCR from overlapping regions from PBMC DNA. For 5′ end amplification of R-U5 region, 4F (5′-CCAGTCATCCGATAGACTGAGTCGC-3′) (SEQ ID NO: 11) and 1154R can be used for first round and 4F and 770R (5′-TACCATCCTGAGGCCATCCTACATTG-3′) (SEQ ID NO: 12) can be used for second round. For regions including gag-pro and partial pol, 350F(5′-GAGTTCGTATTCCCGGCCGCAGC-3′) (SEQ ID NO: 13) and 5135R (5′- CCTGCGGCATTCCAAATCTCG-3′) (SEQ ID NO: 14) can be used for first round followed by second round with 419F and 4789R (5′-GGGTGAGTCTGTGTAGGGAGTCTAA-3′) (SEQ ID NO: 15). For regions including partial pol and env region, 4166F (5′- CAAGAAGGACAACGGAGAGCTGGAG-3′) (SEQ ID NO: 16) and 7622R (5′- GGCCTGCACTACCGAAAT TCTGTC-3′) (SEQ ID NO: 17) can be used for first round followed by 4672F (5′-GAGCCACCTACAATCAGACAAAAGGAT-3′) (SEQ ID NO: 18) and 7590R (5′-CTGGACCAAGCGGTTGAGAATACAG-3′) (SEQ ID NO: 19) for second round. For the 3′ end including the U3-R region, 7472F (5′-TCAGGACAAGGGTGGTTTGAG-3′) (SEQ ID NO: 20) and 8182R (5′-CAAACAGCAAAAGGCTTTATTGG-3′) (SEQ ID NO: 21) can be used for first round followed by 7472F and 8147R (5′-CCGGGCGACTCAGTCTATC-3′) (SEQ ID NO: 22) for second round. The reaction mixtures and conditions can be as described above except for the following: For larger fragments, extension can be done at 68° C. for 10 min instead of 72° C. All second round PCR products can be column purified as mentioned above and overlapping sequences can be determined with internal primers. Nested RT-PCR for gag sequences can be done as described with modifications. GAG-O-R primer can be used for 1st strand synthesis; cycle conditions can be 52° C. annealing, for 35 cycles. For second round PCR, annealing can be at 54° C. for 35 cycles.

Once nucleic acids have been amplified by PCR, standard sequencing techniques can be used to determine the nucleic acid sequence thereof Standard in silico translation techniques can be used to determine amino acid sequences from nucleic acid sequences.

### Example 3

This example describes the methods used to analyze the relatedness of viral isolates.

Phylogenetic Analysis: Sequences can be aligned using ClustalX Clustal alignments can be imported into MEGA4 to generate neighbor joining trees using the Kimura 2-parameter plus Γ distribution (K80+Γ) distance model. Free parameters can be reduced to the K80 model, and a values can be estimated from the data set using a maximum likelihood approach in PAUP*4.0 (Sinauer Associates, Inc. Publishers, Sunderland, Mass., USA). The bootstrap consensus tree inferred from 1000 replicates is taken to represent the evolutionary history of the taxa analyzed. Accession numbers from GenBank (http://www.ncbi.nlm.nih.gov/Genbank): FLV (NC_{—}001940), MoMLV (NC_{—}001501), XMRV VP35 (DQ241301, SEQ ID NO: 163)), XMRV VP42 (DQ241302, SEQ ID NO: 164), XMRV VP62 (EF185282, SEQ ID NO: 162). Genomic Nonecotropic MLV Provirus Sequences can be downloaded from PLOS Genetics 3(10): e183.

### Example 4

This example describes how sequence variation in the XMRV gag gene allows identification of three distinct XMRV subgroups. Unless otherwise described, the methods used in this example are as in Examples 1-3.

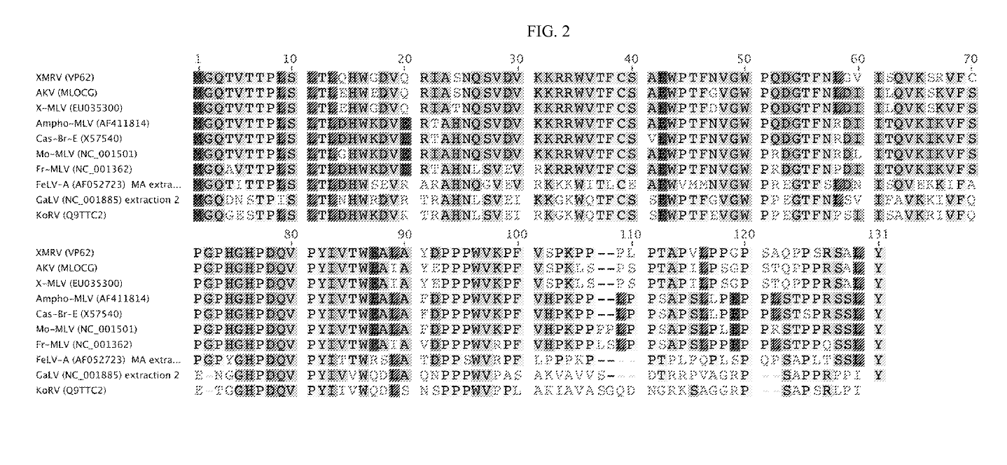

To investigate the diversity of XMRV sequences, peripheral blood mononuclear cells (PBMCs) from XMRV-infected individuals were isolated, and the sequence of the region of gag that encodes the core protein matrix (MA) was determined using nested RT-PCR. This protein is the most diverse of the gag proteins of gammaretroviruses: sequence analysis of several different murine, feline, and primate gammaretroviruses have revealed low sequence and residue identity (see e.g., FIG. 2). In contrast, the MA sequences of XMRV available on GenBank show significant conservation, differing by 0-2 of 387 (<1%) nucleotides. The present inventors found significant variation from the consensus sequence in MA proteins isolated from patients with CFS.

To further investigate the genetic diversity of additional XMRV isolates, RNA was isolated directly from the PBMCs of XMRV-infected individuals and regions encoding the MA protein were amplified by nested RT-PCR (see e.g., FIG. 5A). Comparison of the sequences amplified from 17 individuals revealed a significant amount of variation in this region (see e.g., FIG. 5B). All sequences analyzed directly from XMRV-infected subjects differed by at least one nucleotide from the XMRV reference strain VP62

Nucleotide changes identified in clinical isolates of XMRV, with reference to sequence numbering of VP62, Accession number EF185282.1 (SEQ ID NO: 162) (see e.g., Table 1). Nucleotide substitutions were also reported according to position in VP62, Accession number DQ399707.1 (SEQ ID NO: 1) (see e.g., Table 1). Amino acid changes determined with respect to alignment SEQ ID NO: 162 (see e.g., FIG. 5B, line 1). Overall, 15/327 residues had nucleotide substitutions relative to VP62, and all but two of these changes were observed in two or more of the isolates examined. In addition, 7 of the 17 samples had a 21 by deletion from nt 127- 147.

Analysis of the MA sequences revealed the variants could be classified into three subgroups (see e.g., FIG. 5C). These subgroup delineations are supported by unrooted neighbor-joining analysis of the MA nucleotide sequence fragment. Seven of the sequences, which fell into subgroup A, are closely related to the previously published sequences of XMRV in this region (see e.g., FIG. 5C, lines 2-8, compare with line 1). At most, this group differed by 3 nucleotides from the reference strain VP62; one resulted in a synonymous change (i.e., the same residue was encoded). and two were non-synonymous (see e.g., TABLE 1). The non-synonymous substitution, nt 178: G→A, is present in all of the sequences in subgroup A (G178A), and has also been previously reported to be present in other XMRV sequences (see e.g., TABLE 1).

Eight of the seventeen (8/17) sequences analyzed fell into a second group (subgroup B), all of which had a 21 by deletion, resulting in an in-frame deletion of seven amino acid residues. All sequences in subgroup B also had seven specific nucleotide substitutions relative to the sequence of the XMRV reference strain (nt 75: C→T, nt 85: G→A, nt 91: A→G, nt 92: A→G, nt 304: C→G, nt 315: C→T, and nt 316: C→T) (see e.g., FIG. 1B lines 9-17), of which were four were synonymous and three were non-synonymous changes (see e.g., TABLE 1).

Subgroup C contained two sequences and was characterized by three unique nucleotide substitutions (nt 106: G→A, nt 175: G→A, and nt 192: C→T) (see e.g., FIG. 4, lines 17 and 18; and TABLE 1), of which two were synonymous and one was a non-synonymous change. This group also had three nucleotide substitutions relative to VP62 that were in common with members of groups A and B (nt 92: A→G, nt 178: G→A, nt 325: A→G).

To gain insight into whether the variation observed in the XMRV sequences could be tolerated by the MA protein and persist in nature, MA protein sequences of gammaretroviruses from other mus muculus and other species examined Alignment of MA proteins of other members gammaretrovirus genus revealed that 5 of the 6 amino acid changes in the XMRV variants are present in other infectious gammaretroviruses (see e.g., TABLE 4 and FIG. 2).

### Example 5

This example describes analysis of XMRV MA sequences in lymphocytes following ex vivo XMRV culture. Unless otherwise described, methods are as in Examples, 1-4.

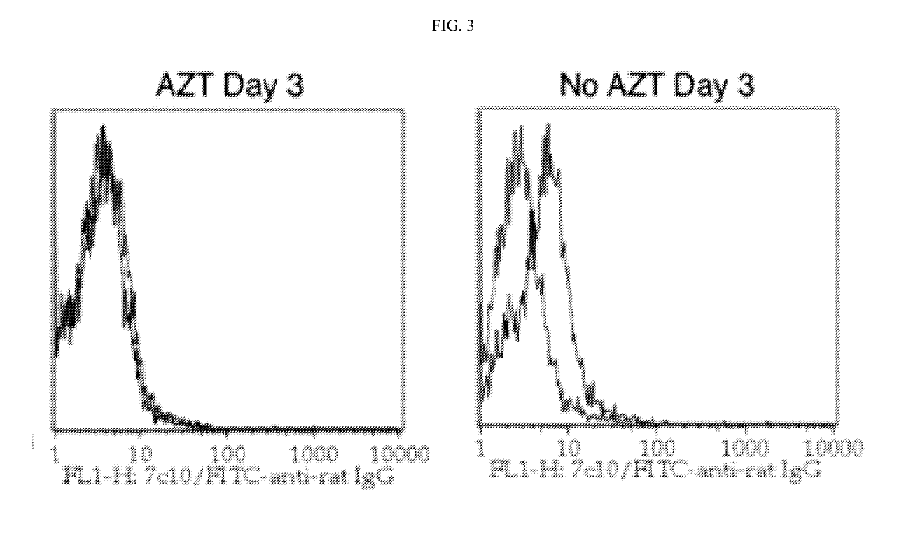

XMRV RNA could not always be detected in the PBMCs of subjects from which infectious virus had been isolated from plasma. This suggests that the virus is expressed at very low frequency in PBMCs isolated directly from infected individuals. We have observed that culturing these PBMCs under conditions that induce activation of T cells increases the frequency of XMRV detected by RT-PCR in the cells maintained in culture. This increase appears to be dependent on the spread of the virus, since the addition of a reverse transcriptase inhibitor to the cultures prior to activation prevents the increase XMRV expression, as measured by cell surface expression of Env (see e.g., FIG. 3). To biologically increase the level of XMRV and increase the probability that XMRV sequences could be detected by PCR, PBMCs were cultured under conditions that activated T cells for 7-10 days, the RNA isolated, and nested RT-PCR analysis performed as described above.

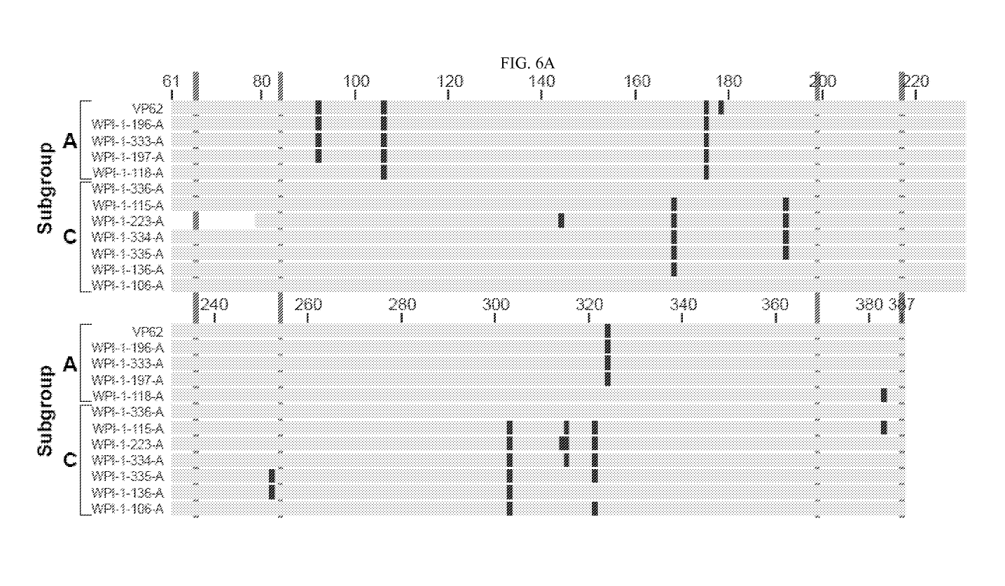

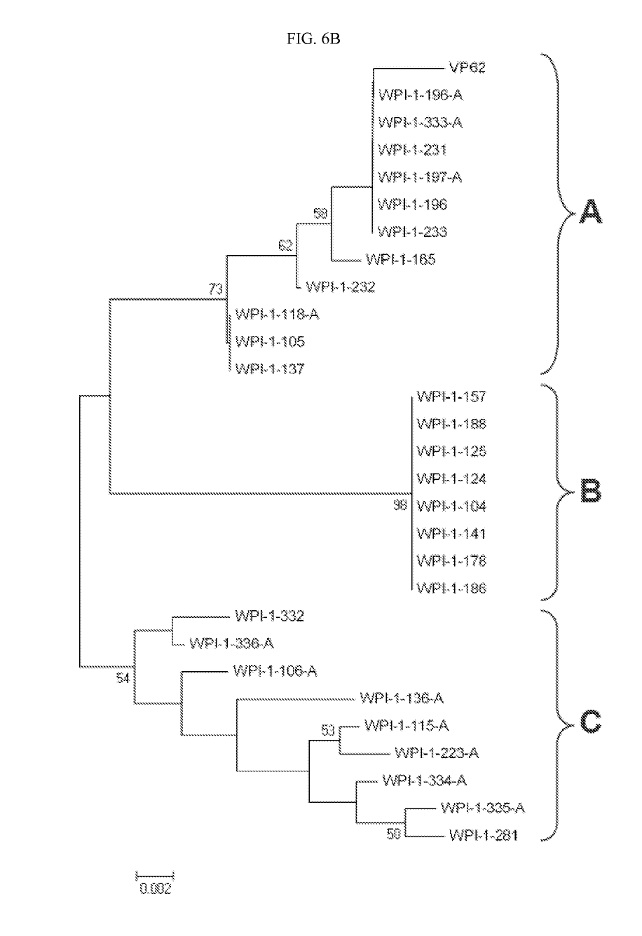

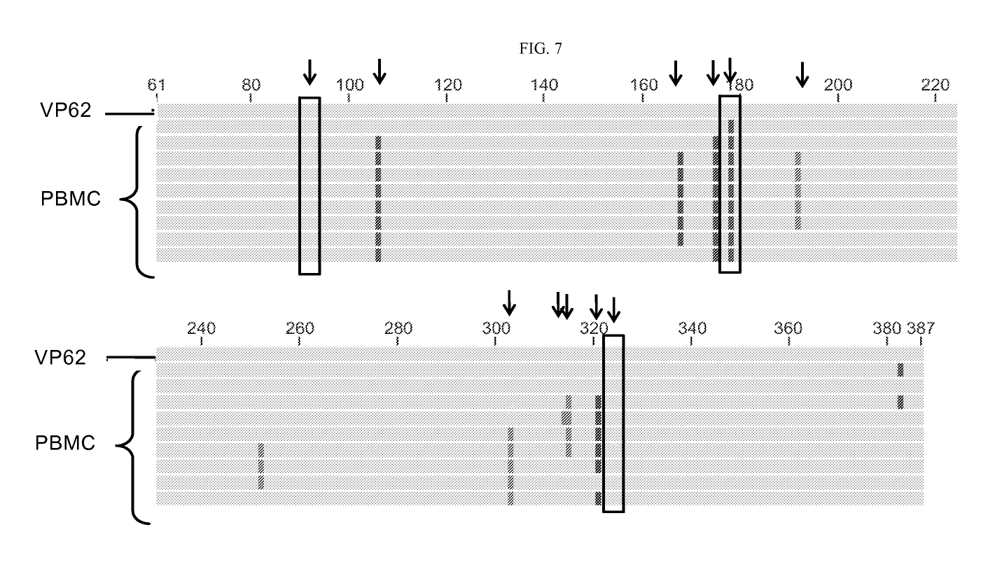

All MA sequences amplified following ex vivo culture could be classified into two out of the three subgroups observed in the analysis of RNA from unactivated PBMCs. Sequences for 4/11 individuals were similar to the previously published sequences (subgroup A) (see e.g., FIG. 6A, lines 2-5). Sequences amplified from another 6 individuals fell into subgroup C(see e.g., FIG. 6A, lines 6-12). Unrooted neighbor-joining analysis of nucleotide sequences direct from subject PBMCs and after ex vivo culture reflected the variability noted in the sequence analysis and confirmed that post-culture, only variants A and C can be detected (see e.g., FIG. 6B).

### Example 6

This example provides evidence of multiple variants in a single XMRV-infected individual. Unless otherwise described, methods are as in Examples 1-5.

None of the sequences amplified following ex vivo culture were similar to the sequences of subgroup B. One explanation for this would be that the PBMCs contained multiple strains of XMRV and, because of differences in replication capacity or tropism, the major variant present following spread in the cultures differed from the major variant present in unstimulated PBMCs of infected individuals. Reexamination of direct sequencing data obtained from unactivated PBMCs suggested that several of the sequence chromatograms might reflect the presence of more than one virus (See e.g., FIG. 10, note the occasional noisiness of the sequence chromatograph, which indicates distinct sequence populations).

Analysis with the Mutation Surveyor software program, which can deconvolute overlapping sequences, showed the presence of a subgroup B sequence and a subgroup C sequence in three isolates. WPI-1-104 had ˜60% subgroup B and ˜40% subgroup C; WPI-1-136 had ˜80% subgroup B and ˜20% subgroup C; and WPI-1-115 had ˜20% subgroup B and ˜80% subgroup C.

For two sample (WPI-1-115, WPI-1-136), sequences were obtained following activation and culture of PBMCs. In both cases, the viral sequences detected from amplification of RNA were subgroup C (see e.g., FIG. 6A, lines 6 and 10) following ex vivo activation and culture of T cells, suggesting that subgroup B variants have a decreased replicative capacity.

### Example 7

This example describes and characterizes the sequence diversity of XMRV isolates. Methods are as in Examples 1-6, unless otherwise described.

Previous comparison of the major coding regions of XMRV with MLV sequences indicated that, while the pol and env sequences of XMRV cluster with X-MLVs, the gag region of XMRV clusters with polytropic (P-MLVs) and modified polytropoic (Pm-MLV) viruses as well as X-MLVs (Urisman et al. 2006 PLoS Pathog 2(3), e25).

As shown herein, comparison of XMRV MA subgroup A sequnces in GenBank indicates that, similar to previously published XMRV sequences, subgroup A is most closely related to a X-MLVs, but also clusters with several P-MLV and Pm-MLV sequences. As seen in previously published sequences, none of the group A variants are identical to any known X-MLV sequence.

In contrast, comparison of XMRV MA sequences from subgroup B with sequences in GenBAnk revealed 100% identity with a P-MLV, mobilized endogenous retrovirus clone 51 (see Evans et al. 2009 J Virol 83(6), 2429-2435). Clone 51 is expressed in certain strains of mice but contains several deletions and is not infectious. But when mice expressing clone 51 are infected with an ecotropic MLV (Fr-MLV), clone 51 genomes can be packaged into the Fr-MLV virion and transferred to rodent cell lines (Evans et al. 2009 J Virol 83(6).

Subgroup C MA sequences are closely related to the MA of both P-MLV and Pm-MLV sequences. One variant in subgroup C (WP-1-281) was identical on a nucleotide level to both an endogenous Pm-MLV on chromosome 7, and to an expressed endogenous P-MLV with large deletions in gag and pol (Rmcf provirus) (see Jorgensen et al. 1992 J Virol 66(7) 4479-4487). Others in this group differed in nucleotide sequence from sequenced variants. But these substitutions were generally synonymous ands resulted in conservation of the MA sequences at the amino acid level. Thus, in this study, MA sequences of XMRV subgroup B and C are more homologous to known endogenous sequences that the XMRV subgroup A viruses.

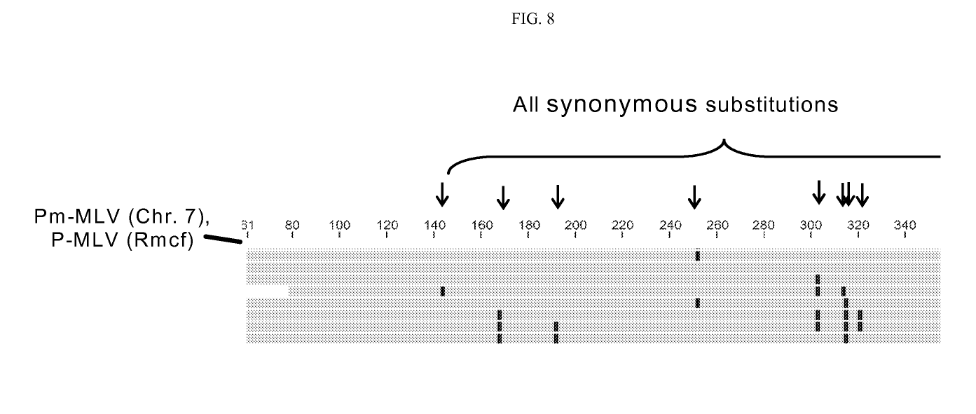

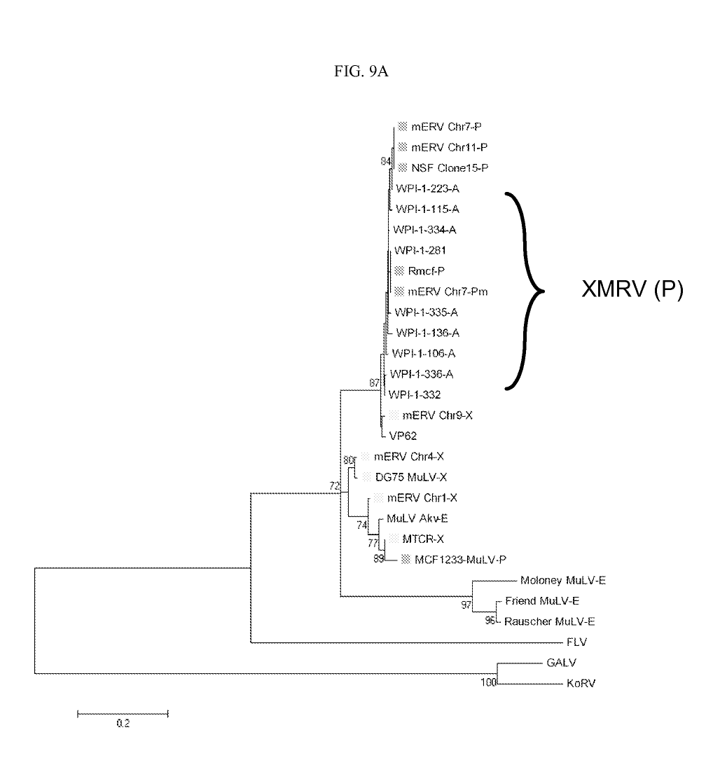

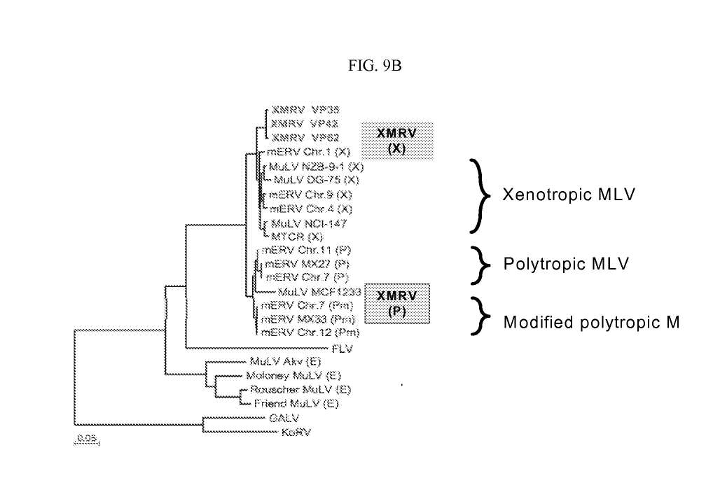

XMRV sequences were also analyzed to determine their relatedness to MLVs generally. The consensus sequence for the N-terminus of the Env protein of XMRV is similar to the Env protein of Spleen Focus Forming Virus (SFFV; see e.g., FIG. 4), consistent with the inventors' previous use of antibodies originally raised against SFFV to recognize XMRV. FIG. 8 shows the nucleotide variation between sequences encoding MA protein in several XMRV isolates, and in two other MLVs. FIGS. 9 is a phylogenetic tree showing the relatedness of a number of separate XMRV isolates to each other and to other gammaretroviruses. FIGS. 11A-B show the sequence variation in clinical isolates of XMRV, the XMRV reference strain VP62, and other MLVs.

### Example 8

This example shows that APOBEC may be responsible for variation in clinically isolated XMRV sequences.

APOBEC3 restriction factors are cellular proteins capable of blocking replication of many retroviruses. Others (Groom et al., PNAS 2010, 107(11): 5166-5171; Stieler and Fischer, PLoS One 2010, el1738; Paprotka et al., J Virology 2010, 84(11):5719-5729) have shown that expression of human APOBEC3G (“hA3G”) in cells infected with XMRV dramatically reduced viral titer and caused G-to-A hypermutation of the viral DNA. However, it is not clear that APOBEC restriction factors would regulate XMRV infection: APOBECs are generally expressed at only low levels even in those cells which do express them; XMRV normally infects a subset of lymphocytes that are known not to express APOBEC proteins; and XMRV has specific countermeasures to evade hA3G. To determine if hA3G is a natural regulator of XMRV infection, then, the present inventors looked for hallmarks of APOBEC activity on XMRV sequences isolated from peripheral blood mononuclear cells (“PBMCs”) from XMRV-infected individuals.

Experiments examined the XMRV derived from PBMCs from infected individuals for evidence of APOBEC-associated hypermutation using methods as described in Examples 1-8, unless otherwise specified. PBMCs were isolated from XMRV-infected individuals, and B “cell lines” were generated from the PBMCs. XMRV was then isolated from the cell lines and the DNA was cloned and sequenced.

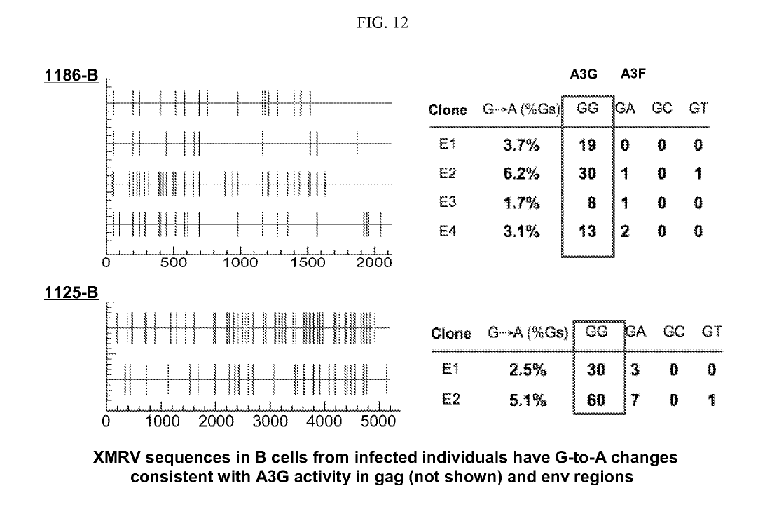

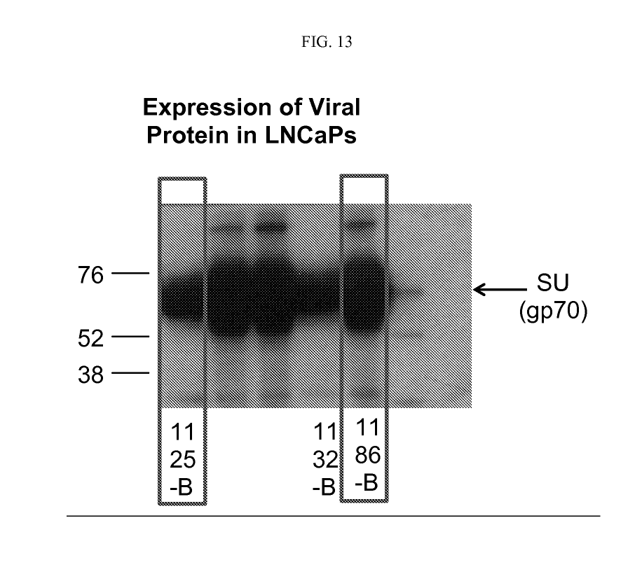

Data not shown and FIGS. 12-13 show that the XMRV sequences from infected individuals have G-to-A changes consistent with hA3G activity in both Gag and Env coding regions. The data shows a clear preference for substitutions at GG dinucleotides, consistent with the A3G form of APOBEC, as opposed to the A3F form, which targets GA dinucleotides. These highly mutated XMRV isolates were nevertheless able to infect LNCaP cells at similar rates as wild-type XMRV (data not shown), and were able to produce translatable XMRV proteins (eg, FIG. 13). The data suggest, therefore, that APOBEC may be responsible for the high amount of sequence diversity between clinically isolated XMRV sequences.

### Example 9

This example shows the variation in clinically isolated XMRV sequences. Methods are as in Examples 1-8 unless otherwise specified.

XMRV was isolated from samples from XMRV-infected subjects and amplified and sequenced according to standard methods. Sample number 1253 was identified as a P-type XMRV.

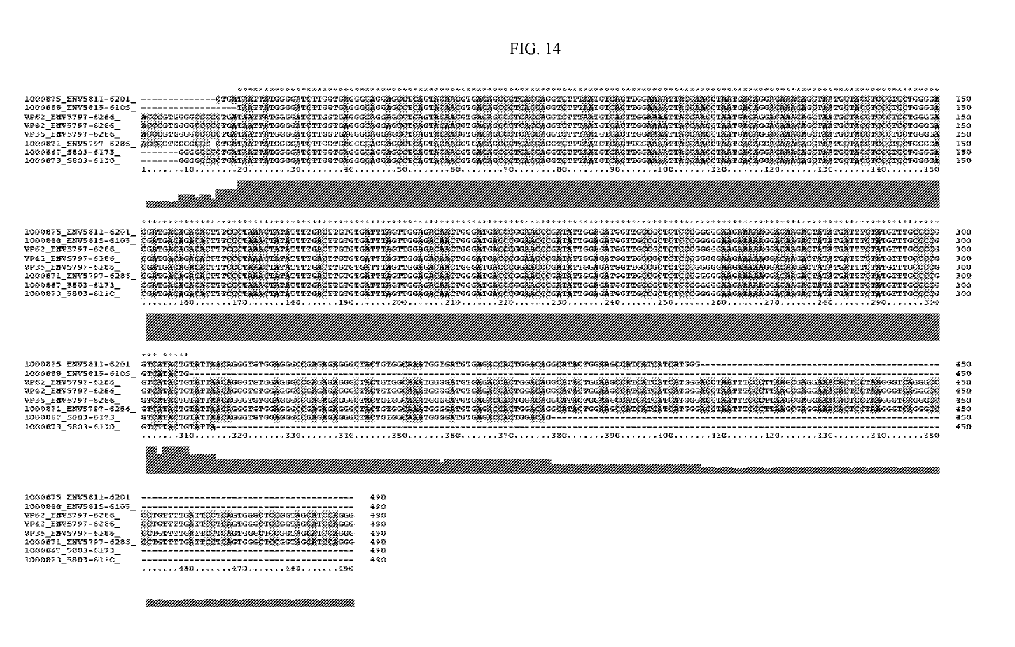

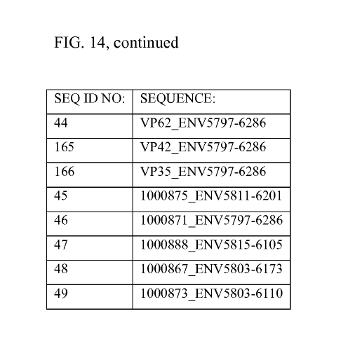

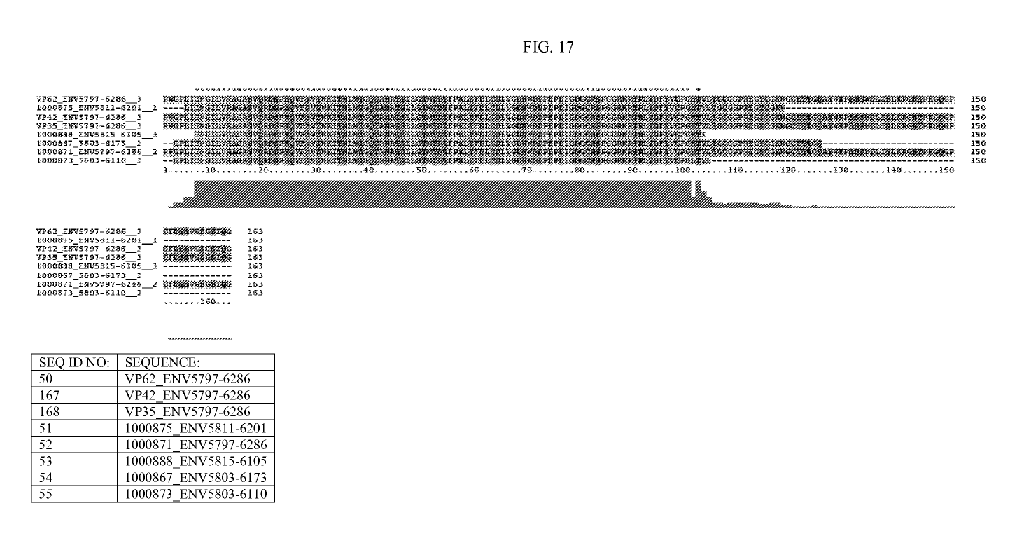

FIGS. 14-19 are sequence alignments of sequences from XMRV clinical isolates. The sequence alignments show variation in polynucleotide sequences (see e.g., FIGS. 14-16) and polypeptide sequences (see e.g., FIGS. 17-19). Numbering of nucleotide or amino acid positions is relative to VP62 (SEQ ID NO: 1). Nucleotide and amino acid changes from reference VP62, SEQ ID NO: 1, are shown in TABLE 2.

### Example 10

This example shows that XMRV isolated from individuals with prostate cancer and CFS form a distinct phylogenetic unit, distinct from all mouse xenotropic viruses. Methods are according to Examples 1-9, unless otherwise specified.

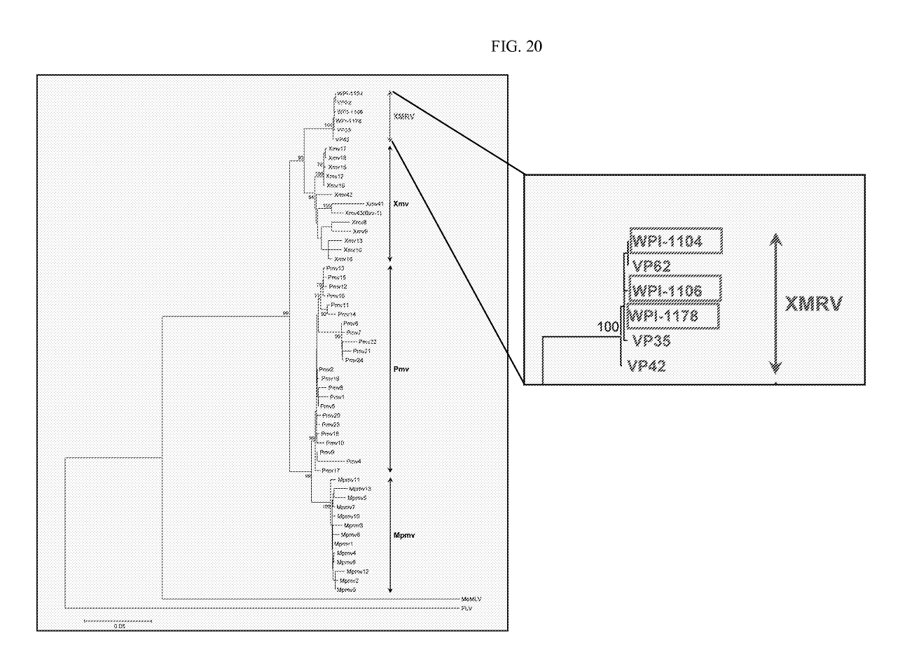

XMRV was isolated from subjects with prostate cancer and from subjects diagnosed with or showing symptoms of CFS. The XMRV from the isolates were amplified and sequenced according to standard methods. A phylogenetic tree was built with the sequencing data (see e.g., FIG. 20).

The clinical XMRV isolates (WPI-1104, WPI-1106, and WPI-1178), as well as three XMRV reference sequences (VP62, SEQ ID NO: 1; VP42, SEQ ID NO: 164; and VP35, SEQ ID NO: 163) all cluster together, and away from all other murine xenotropic viruses.

### Example 11

This example shows that SU sequences of viruses transmitted from the plasma of UK ME/CFS patients to LNCaP cells shares homology with XMRV and not with polytropic MLV. Unless otherwise indicated, methods are as in Examples 1-11.

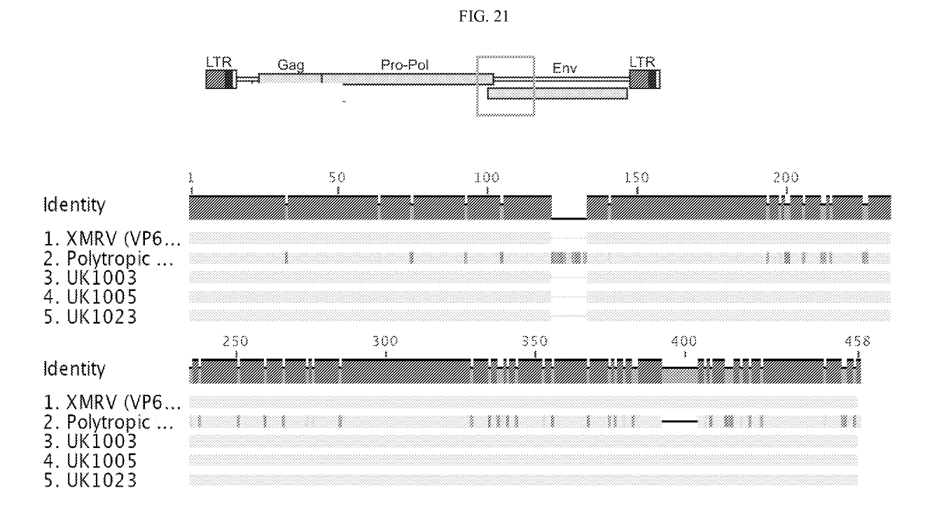

FIG. 21 shows sequence alignments of sequences from viruses from ME/CFS patients from the UK, which were able to co-cultured with LNCaP cells. The sequences are more similar to the VP62 XMRV reference sequence than to the polytropic MLV reference sequence.

### Example 12

This example shows that XMRV clinical isolates from a Norwegian ME/CFS cohort show variation. Unless otherwise indicated, methods are as described in Examples 1-12.

In this study, patients were selected with strict criteria for illness: they were either homebound or bedridden because of ME/CFS. Blood was collected from the patients at home. Thirty-nine samples that were XMRV-positive were sequenced. Most of the samples show a 100% sequence match to VP62. However, twenty-three samples comprised XMRV with different (non-VP62) sequences. One sample comprised a virus with a sequence closely related to Mus musculus mobilized endogenous polytropic provirus clone 15.

### Example 13

This example shows that XMRV in CFS patients in Germany is distinguished from the XMRV produced by the 22Rv1 cell line.

22Rv1 is a human prostate carcinoma epithelial cell line derived from a xenograft that was serially propagated in mice after castration-induced regression and relapse of the parental, androgen-dependent CWR22 xenograft. Recently, it has been shown that 22Rv1 prostate carcinoma cells produce high-titer of XMRV.

In this blinded study, XMRV was detected by: PCR was performed directly on patient plasma; serological assay; and isolation of virus. TABLE 6 shows the results from different types of assays for the presence of XMRV, and the results of experiments to determine the sequences of the isolated viruses.

TABLE 6
Results of assays for XMRV in the study of German CFS patients.

| Sample | Antibody | Plasma PCR | 100% Sequence Homology |
|---|---|---|---|
| 3101 HD6E | − | + | 22Rv1 |
| 3102 HD7E | − | + | 22Rv1 |
| 3103 HD8E | − | + | 22Rv1 |
| 1748 HD9E | − | + | VP62 |
| 1716 HD18E | − | + | VP62 |
| 1723 HD19E | + | + | VP62 |

### Example 14

This example shows that clones of Env sequences amplified from PBMCs from subject WPI-1104 are similar to sequences from polytropic MLVs. Methods are as in Examples 1-14 unless otherwise specified.

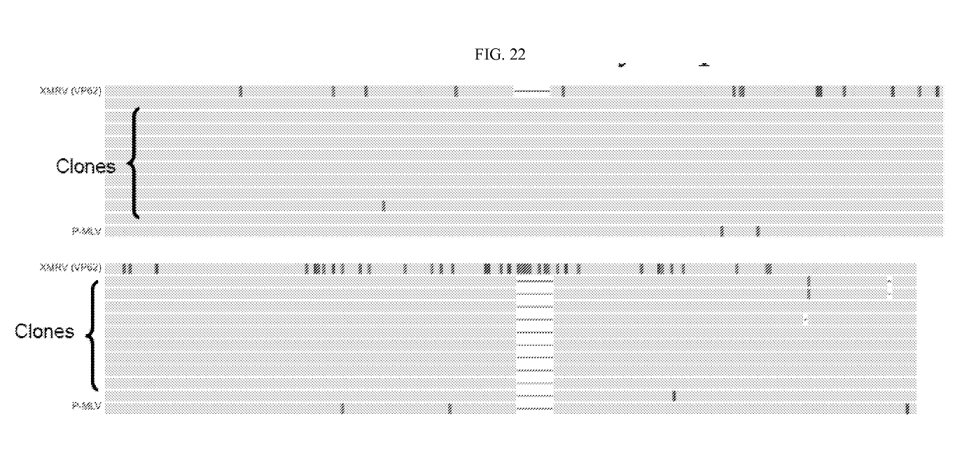

In this example, virus was cultured from PBMCs from subject WPI-1104. The cultured viruses were then used to infect LNCaP cells, and virus was reisolated from those cells and the polynucleic acids were sequenced. Greater than 50 cultures of LNCaP cells have been infected using WPI-1104-derived virus. A representative selection of resulting sequence data is shown in an alignment in FIG. 22. The sequences isolated from this subject are more closely related to polytropic MLVs than to VP62.

This finding suggests that some XMRV-type viruses may replicate more efficiently in LNCaP cells.

What is claimed is:

1. An isolated Xenotropic Murine Leukemia Virus-Related Virus (XMRV) polynucleotide comprising:
(i) a nucleic acid sequence according to SEQ ID NO: 1 and one or more nucleotide sequence changes selected from the group consisting of C80T, G90A, A96G, A97G, G111A, A137-157 deletion, T173C, G180A, G183A, C197T, C247T, C257T, C308T, C308G, C319T, C320T, T326C, A329G, C715T, T791G, A804G, T816Del, A856G, A665Del, T691G, G790A, T791G, T796C, G807Del, A840G, A873G, A875G, C903T, T963G, C5810Del, A6101T, G6154T, G7421A, A7459C, and an insertion at nucleotide position 7322 having a sequence of SEQ ID NO: 179, or a detectable fragment thereof;
(ii) a nucleic acid sequence having at least about 95% sequence identity to a sequence of (i) and having an XMRV associated function or activity; or
(iii) a functional fragment of a sequence of (i) or (ii) and having an XMRV associated function or activity.

2. The isolated XMRV polynucleotide of claim 1, wherein the XMRV associated function or activity is selected from the group consisting of:
(i) encoding of an RNA active gammaretrovirus core encapsidation signal;
(ii) formation of XMRV virion particles;
(iii) stimulation of a cytokine or chemokine signature indicative of an immune response in a subject in vivo;
(iv) formation of anti-XMRV antibodies according to an in vivo humoral immune response in a subject;
(v) similar, same, or greater ex vivo fitness compared to an XMRV control or strain according to a growth competition assay;
(vi) ability to infect a cell in a modified Derse assay;
(vii) reverse transcriptase activity;
(viii) ability to immortalize or modify a phenotype of a primary cell or cell culture;
(ix) ability to induce cell syncytia or cell death on exposure or infection of cultured primary cells or co-cultured indicator cells;
(x) ability to form plaques in cell culture on exposure or infection; and
(xi) similar, same, or lower tissue culture infective dose (TCID₅₀) compared to an XMRV control or strain.

3. An isolated Xenotropic Murine Leukemia Virus-Related Virus (XMRV) Envelope polypeptide comprising:
(i) an amino acid sequence according to SEQ ID NO: 160 and one or more amino acid sequence changes selected from the group consisting of H116L, G134Stop, an insertion between amino acid positions 517-518 having an amino acid sequence of SEQ ID NO: 180, E535K, D549A, and R568G, or a detectable fragment thereof;
(ii) an amino acid sequence having at least about 95% sequence identity to a sequence of (i) and having an XMRV associated function or activity; or
(iii) a functional fragment of a sequence of (i) or (ii) and having an XMRV associated function or activity.

4. The isolated XMRV Envelope polypeptide of claim 3, wherein the XMRV associated function or activity is selected from the group consisting of:
(i) an extracellular topological domain at amino acid positions 34-585; a helical transmembrane region at amino acid positions 586-606; a cytoplasmic topological domain at amino acid positions 607-640; a receptor-binding domain at amino acid positions 32-237; a fusion peptide region at amino acid positions 447-467; an immunosuppression region at amino acid positions 513-529; a coiled coil region at amino acid positions 490-510; a CXXC motif at amino acid positions 311-314; a CX6CC motif at amino acid positions 530-538; a YXXL motif containing an endocytosis signal at amino acid positions 630-633; a Pro-rich region at amino acid positions 234-283; a cleavage site at amino acid position 444-445; and a cleavage site at amino acid position 624-625;
(ii) an ability for the Envelope polypeptide to be cleaved to a surface protein (SU), a transmembrane protein (TM), and an R-protein;
(iii) SU activity, TM activity, or R-peptide activity;
(iv) an association of a trimer of SU-TM heterodimers attached by a labile interchain disulfide bond;
(v) stimulation of a cytokine or chemokine signature indicative of an immune response in a subject in vivo; and
(vi) formation of anti-XMRV antibodies according to an in vivo humoral immune response in a subject.

5. An isolated Xenotropic Murine Leukemia Virus-Related Virus (XMRV) Gag-Pol polypeptide comprising:
(i) an amino acid sequence according to SEQ ID NO: 161 and one or more amino acid sequence changes selected from the group consisting of K31G, K31R, V36I, a 7 amino acid deletion from aa126-146, a 7 amino acid deletion from aa132-152, G59S, V60I, P105L, S27P, K31R, S62P; K65N, K65N and a downstream reading frame change according to SEQ ID NO: 105, and H76R, or a detectable fragment thereof;
(ii) an amino acid sequence having at least about 95% sequence identity to a sequence of (i) and having an XMRV associated function or activity; or
(iii) a functional fragment of a sequence of (i) or (ii) and having an XMRV associated function or activity.

6. The isolated XMRV Gag-Pol polypeptide of claim 5, wherein the XMRV associated function or activity is selected from the group consisting of:
(i) a peptidase A2 domain at amino acid position 559-629, a reverse transcriptase domain at amino acid position 739-930, an RNase H domain at amino acid position 1172-1318, an integrase catalytic domain at amino acid position 1442-1600, a CCHC-type domain at amino acid position 500-517, a coiled coil at amino acid position 436-476, a PTAP/PSAP motif at amino acid position 109-112, a LYPX(n)L motif at amino acid position 128-132, a PPXY motif at amino acid position 161-164, a Pro-rich region at amino acid position 71-191, or Pro-rich region at amino acid position 71-168, a protease active site at amino acid position 564, a magnesium metal binding catalytic site for reverse transcriptase activity at amino acid positions 807, 881, or 882, a magnesium metal binding site for RNase H activity at amino acid positions 1181, 1219, 1240, or 1310, a magnesium metal binding catalytic site for integrase activity at amino acid positions 1453 or 1512, and a cleavage site by viral protease p14 at amino acid positions 129-130, 213-214, 476-477, 532-533, 657-658, or 1328-1329;
(ii) an ability for the Gag-Pol polypeptide to be cleaved to a matrix protein p15, a RNA-binding phosphoprotein p12, a capsid protein p30, a nucleocapsid protein p10, a protease p14, a reverse transcriptase/ribonuclease H, and an integrase p46;
(iii) matrix protein p15 activity, RNA-binding phosphoprotein p12 activity, capsid protein p30 activity, nucleocapsid protein p10 activity, protease p14 activity, reverse transcriptase/ribonuclease H activity, or integrase p46 activity;
(iv) stimulation of a cytokine or chemokine signature indicative of an immune response in a subject in vivo; and
(v) formation of anti-XMRV antibodies according to an in vivo humoral immune response in a subject.

7. A method of detecting a strain of Xenotropic Murine Leukemia Virus-Related Virus (XMRV) in a sample comprising detecting presence, absence, or quantity of the XMRV polynucleotide or polypeptide of any one of claims 1-6, or an immune response of a subject thereto, in the sample.

8. The method of claim 7, wherein:
the sample is selected from the group consisting of a blood sample, a serum sample, a plasma sample, a cerebrospinal fluid sample, and a solid tissue sample; or
the sample comprises cells selected from the group consisting of fibroblasts, endothelial cells, peripheral blood mononuclear cells, and haematopoietic cells, or a combination thereof.

9. The method of any one of claims 7-8, wherein detecting presence, absence, or quantity of an XMRV strain in a sample comprises:
contacting the sample and at least one probe that binds to at least one XMRV strain polypeptide, or detectable fragment thereof, under conditions sufficient for formation of a complex comprising the at least one probe and the least one polypeptide or fragment if present in the sample; and
detecting presence, absence or quantity of the complex comprising the at least one probe and the at least one polypeptide or fragment.

10. The method of claim 9, wherein one or more of the following is satisfied:
(i) the at least one probe is a polyclonal antibody, a monoclonal antibody, an Fab fragment an antibody, an antigen-binding fragment of an antibody, an aptamer, or an avimer, optionally selected from the group consisting of an anti gp 55 Env antibody, monoclonal antibody MAb 7C10, a monclonal antibody against p30 gag, and a polyclonal antibody against mouse xenotropic virus;
(ii) detecting presence, absence or quantity of the complex comprises at least one of an immunoprecipitation assay, an ELISA, a radioimmunoassay, a Western blot assay or a flow cytometry assay;
(iii) contacting the sample and the at least one probe comprises contacting the sample with a solid surface that binds the at least one XMRV polypeptide and subsequently contacting the surface with the at least one probe; or contacting the sample with a solid surface that binds the at least one XMRV polypeptide, subsequently contacting the surface with the at least one probe, and quantifying the at least one probe bound to the surface, wherein the solid surface is selected from the group consisting of a plate, a bead, a dip stick, a test strip, membrane and a microarray;
(iv) the at least one probe comprises a label, detecting presence, absence or quantity of a complex comprises quantifying the label, and the label is selected from the group consisting of a radioisotope, a chromogen, a chromophore, a fluorophore, a fluorogen, an enzyme, a quantum dot and a resonance light scattering particle; or
(v) detecting presence, absence or quantity of a complex comprises contacting the complex and at least one secondary probe and detecting presence, absence or quantity of the at least one secondary probe, wherein at least one secondary probe binds the at least one probe or the at least one XMRV polypeptide.

11. The method of any one of claims 7-8, wherein detecting presence, absence, or quantity of an XMRV strain in a sample is according to a serocoversion assay comprising:
contacting the sample and at least one XMRV antigen under conditions sufficient for formation of a complex between the at least one XMRV antigen and an immunopeptide specific for an XMRV strain if the immunopeptide is present in the sample; and
detecting presence, absence or quantity of the complex comprising the XMRV antigen and the anti-XMRV immunopeptide;
wherein the XMRV antigen comprises the XMRV polynucleotide or polypeptide, or a fragment thereof

12. The method of claim 11, wherein one or more of the following is satisfied:
(i) detecting presence, absence or quantity of the complex comprises contacting the complex comprising the XMRV antigen and the anti-XMRV immunopeptide of the sample with at least one probe directed against a serum retroviral immunopeptide or the XMRV antigen under conditions sufficient for formation of an complex comprising the at least one probe and the XMRV immunopeptide or the XMRV antigen; and detecting presence, absence or quantity of the probe;
(ii) contacting the sample and at least one XMRV antigen comprises contacting the sample with a solid surface comprising a bound at least one XMRV antigen and detecting presence, absence or quantity of the complex comprising the XMRV antigen and the anti-XMRV immunopeptide; or contacting the sample with a solid surface comprising a bound at least one XMRV antigen, contacting the surface with at least one probe directed against a serum retroviral immunopeptide under conditions sufficient for formation of an complex comprising the at least one probe and the XMRV immunopeptide, and detecting presence, absence or quantity of the probe, wherein the solid surface is selected from the group consisting of a plate, a bead, a dip stick, a test strip, membrane and a microarray; or
(iii) the at least one XMRV antigen comprises a contiguous sequence of at least about 4 amino acids of the XMRV polypeptide comprising at least one of the amino acid sequence changes.

13. The method of any one of claims 7-8, wherein detecting presence, absence, or quantity of an XMRV strain in a sample comprises:
contacting the sample and at least one nucleobase polymer under conditions sufficient for hybridization to occur between the at least one nucleobase polymer and a polynucleotide of a XMRV strain, or complement thereof, if present in the sample; and
detecting presence, absence or quantity of a hybridization complex comprising the nucleobase polymer and the XMRV polynucleotide, or complement thereof;
wherein the at least one nucleobase polymer comprises a sequence that hybridizes to a nucleic acid sequence comprising at least about 10 contiguous nucleotides of a polynucleotide of an XMRV strain, or complement thereof.

14. The method of claim 14, wherein one or more of the following is satisfied:
(i) the at least one nucleobase polymer comprises a sequence that hybridizes to a nucleic acid sequence comprising at least about 10 contiguous nucleotides of an XMRV polynucleotide comprising at least one of the nucleic acid sequence changes, or complement thereof;
(ii) the conditions sufficient for hybridization to occur consists of high stringency hybridization conditions;
(iii) the nucleobase polymer comprises DNA, RNA, or a nucleic acid analogue;
(iv) the nucleobase polymer further comprises a label selected from the group consisting of a radioisotope, a chromogen, a chromophore, a fluorophore, a fluorogen, an enzyme, a quantum dot and a resonance light scattering particle, and detecting presence, absence or quantity of the hybridization complex comprises detecting presence, absence or quantity of the label; or
(v) detecting presence, absence or quantity of the hybridization complex comprises a hybridization assay selected from the group consisting of a Southern hybridization assay, a Northern hybridization assay, a dot-blot hybridization assay, a slot-blot hybridization assay, a Polymerase Chain Reaction (PCR) assay and a flow cytometry assay, optionally, the PCR assay comprising a quantitative real time polymerase chain reaction assay.

15. The method of any one of claims 7-14, further comprising:
correlating the presence, absence, or quantity of the XMRV strain with an XMRV-related disease or condition;
wherein the sample is a sample of a subject.

16. The method of claim 15, wherein the subject has, is suspected of having, or is at risk for developing an XMRV-related disease or condition; or
the subject exhibits signs or symptoms of an XMRV-related disease or condition.

17. The method of any one of claims 15-16, wherein the XMRV-related disease or condition is selected from the group consisting of prostate cancer, Chronic Fatigue Syndrome, autism, autism spectrum disorders, Gulf War Syndrome, Multiple Sclerosis, Amyotrophic Lateral Sclerosis (ALS), Parkinson's disease, Niemann-Pick Type C Disease, fibromyalgia, chronic Lyme disease, non-epileptic seizures, thymoma, myelodysplasia, Immune Thrombocytopenic Purpura, Mantle Cell Lymphoma, and Chronic Lymphocytic Leukemia lymphoma.

18. The method of any one of claims 15-17, further comprising
(i) selecting or modifying a treatment on the basis of detection of the presence, absence, or quantity of an XMRV strain in a sample of the subject; or
(ii) administering to the subject a therapeutically effective amount of an anti-viral compound if an XMRV strain is detected.


SEQUENCE LISTING (180 sequences)

SEQ ID NO: 1 - DNA, 8185 bp, Xenotropic murine leukemia virus
gcgccagtcatccgatagactgagtcgcccgggtacccgtgttcccaataaagccttttgctgtttgcatccgaagcgtggcctcgctgttccttgggagggtctcctcagagtgattgactacccagctcgggggtctttcatttgggggctcgtccgggattcggagacccccgcccagggaccaccgacccaccgtcgggaggtaagccggccggcgatcgttttgtctttgtctctgtctttgtgcgtgtgtgtgtgtgccggcatctaatcctcgcgcctgcgtctgaatctgtactagttagctaactagatctgtatctggcggttccgcggaagaactgacgagttcgtattcccggccgcagccctgggagacgtcccagcggcctcgggggcccgttttgtggcccattctgtatcagttaacctacccgagtcggactttttggagtggctttgttgggggacgagagacagagacacttcccgcccccgtctgaatttttgctttcggttttacgccgaaaccgcgccgcgcgtctgatttgttttgttgttcttctgttcttcgttagttttcttctgtctttaagtgttctcgagatcatgggacagaccgtaactacccctctgagtctaaccttgcagcactggggagatgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccccctctataaagtccaaacctcctaagccccaggttctccctgatagcggcggacctctcattgaccttctcacagaggatcccccgccgtacggagcacaaccttcctcctctgccagggagaacaatgaagaagaggcggccaccacctccgaggtttcccccccttctcccatggtgtctcgactgcggggaaggagagaccctcccgcagcggactccaccacctcccaggcattcccactccgcatggggggagatggccagcttcagtactggccgttttcctcctctgatttatataattggaaaaataataacccttccttttctgaagatccaggtaaattgacggccttgattgagtccgtcctcatcacccaccagcccacctgggacgactgtcagcagttgttggggaccctgctgaccggagaagaaaagcagcgggtgctcctagaggctagaaaggcagtccggggcaatgatggacgccccactcagttgcctaatgaagtcaatgctgcttttccccttgagcgccccgattgggattacaccactacagaaggtaggaaccacctagtcctctaccgccagttgctcttagcgggtctccaaaacgcgggcaggagccccaccaatttggccaaggtaaaagggataacccagggacctaatgagtctccctcagcctttttagagagactcaaggaggcctatcgcaggtacactccttatgaccctgaggacccagggcaagaaaccaatgtgtccatgtcattcatctggcagtctgccccggatatcggacgaaagttagagcggttagaagatttaaagagcaagaccttaggagacttagtgagggaagctgaaaagatctttaataagcgagaaaccccggaagaaagagaggaacgtatcaggagagaaatagaggaaaaagaagaacgccgtagggcagaggatgagcagagagagagagaaagggaccgcagaagacatagagagatgagcaagctcttggccactgtagttattggtcagagacaggatagacaggggggagagcggaggaggccccaacttgataaggaccaatgcgcctactgcaaagaaaagggacactgggctaaggactgcccaaagaagccacgagggccccgaggaccgaggccccagacctccctcctgaccttaggtgactagggaggtcagggtcaggagcccccccctgaacccaggataaccctcaaagtcggggggcaacccgtcaccttcctggtagatactggggcccaacactccgtgctgacccaaaatcctggacccctaagtgacaagtctgcctgggtccaaggggctactggaggaaagcggtatcgctggaccacggatcgcaaagtacatctggctaccggtaaggtcacccactctttcctccatgtaccagactgcccctatcctctgctaggaagagacttgctgactaaactaaaagcccaaatccactttgagggatcaggagctcaggttgtgggaccgatgggacagcccctgcaagtgctgacagtaaacatagaagatgagtattggctacatgataccaggaaagagccagatgttcctctagggtccacatggctttctgatttccttcaggcctgggcggaaaccgggggcatgggactggcagttcgccaagctcctctgatcatacctctgaaggcaacctctacccccgtgtccataaaacaataccccatgtcacaagaagccagactggggatcaagccccacatacagaggctgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaactgacctggaccagactcccacagggtttcaaaaacagtcccaccctgtttgatgaggcactgcacagagacctagcagatttccggatccagcacccagacttgatcctgctacagtacgtggatgacttactgctggccgccacttctgagcaagactgccaacgaggtactcgggccctattacaaaccctagggaacctcgggtatcgggcctcggccaagaaagcccaaatttgccagaaacaggtcaagtatctggggtatctcctaaaagagggacagagatggctgactgaggccagaaaagagactgtgatggggcagcccactccgaagacccctcgacaactaagggagttcctagggacggcaggcttctgtcgcctctggatccctgggtttgcagaaatggcagcccccttgtaccctcttaccaaaacggggactctgtttaattggggcccagaccagcaaaaggcctatcaagaaatcaaacaggctcttctaactgcccccgccctgggattgccagatttgactaagccctttgaactctttgtcgacgagaagcagggctacgccaaaggcgtcctaacgcaaaaactgggaccttggcgtcggcctgtggcctacctgtccaaaaagctagacccagtggcagctgggtggcccccttgcctacggatggtagcagccattgccgttctgacaaaaaatgcaggcaagctaactatgggacagccgctagtcattctggccccccatgcggtagaagcactggtcaaacaaccccctgaccgttggctatccaatgcccgcatgacccactatcaggcaatgctcctggatacagaccgggttcagttcggaccggtggtggccctcaacccggccaccctgctccccctaccggaaaaggaagccccccatgactgcctcgagatcttggctgagacgcacggaaccagaccggacctcacggaccagcccatcccagacgctgattacacttggtacacagatggaagcagcttcctacaagaaggacaacggagagctggagcagcggtgactactgagaccgaggtaatctgggcgagggctctgccggctggaacatccgcccaacgagccgaactgatagcactcacccaagccttaaagatggcagaaggtaagaagctaaatgtttacactgatagccgctatgccttcgccacggcccatgtccatggagaaatatataggaggcgagggttgctgacctcagaaggcagagaaattaaaaacaagaacgagatcttggccttgctaaaagctctctttctgcccaaacgacttagtataattcactgtccaggacatcaaaaaggaaacagtgctgaggccagaggcaaccgtatggcagatcaagcagcccgagaggcagccatgaaggcagttctagaaacctctacactcctcatagaggactcaaccccgtatacgcctccccatttccattacaccgaaacagatctcaaaagactacgggaactgggagccacctacaatcagacaaaaggatattgggtcctacaaggcaaacctgtgatgcccgatcagtccgtgtttgaactgttagactccctacacagactcacccatctgagccctcaaaagatgaaggcactcctcgacagagaagaaagcccctactacatgttaaaccgggacagaactatccagtatgtgactgagacctgcaccgcctgtgcccaagtaaatgccagcaaagccaaaattggggcaggggtgcgagtacgcggacatcggccaggcacccattgggaagttgatttcacggaagtaaagccaggactgtatgggtacaagtacctcctagtgtttgtagacaccttctctggctgggtagaggcattcccgaccaagcgggaaactgccaaggtcgtgtccaaaaagctgttagaagacatttttccgagatttggaatgccgcaggtattgggatctgataacgggcctgccttcgcctcccaggtaagtcagtcagtggccgatttactggggatcgattggaagttacattgtgcttatagaccccagagttcaggacaggtagaaagaatgaatagaacaattaaggagactttgaccaaattaacgcttgcatctggcactagagactgggtactcctactccccttagccctctaccgagcccggaatactccgggcccccacggactgactccgtatgaaattctgtatggggcacccccgccccttgtcaattttcatgatcctgaaatgtcaaagttaactaatagtccctctctccaagctcacttacaggccctccaagcagtacaacaagaggtctggaagccgctggccgctgcttatcaggaccagctagatcagccagtgataccacaccccttccgtgtcggtgacgccgtgtgggtacgccggcaccagactaagaacttagaacctcgctggaaaggaccctacaccgtcctgctgacaacccccaccgctctcaaagtagacggcatctctgcgtggatacacgccgctcacgtaaaggcggcgacaactcctccggccggaacagcatggaaagtccagcgttctcaaaaccccttaaagataagattaacccgtggggccccctgataattatggggatcttggtgagggcaggagcctcagtacaacgtgacagccctcaccaggtctttaatgtcacttggaaaattaccaacctaatgacaggacaaacagctaatgctacctccctcctggggacgatgacagacactttccctaaactatattttgacttgtgtgatttagttggagacaactgggatgacccggaacccgatattggagatggttgccgctctcccgggggaagaaaaaggacaagactatatgatttctatgtttgccccggtcatactgtattaacagggtgtggagggccgagagagggctactgtggcaaatggggatgtgagaccactggacaggcatactggaagccatcatcatcatgggacctaatttcccttaagcgaggaaacactcctaagggtcagggcccctgttttgattcctcagtgggctccggtagcatccagggtgccacaccggggggtcgatgcaaccccctagtcctagaattcactgacgcgggtaaaagggccagctgggatgcccccaaaacatggggactaagactgtatcgatccactggggccgacccggtgaccctgttctctctgacccgccaggtcctcaatgtagggccccgcgtccccattgggcctaatcccgtgatcactgaacagctacccccctcccaacccgtgcagatcatgctccccaggactcctcgtcctcctccttcaggcgcggcctctatggtgcctggggctcccccgccttctcaacaacctgggacgggagacaggctgctaaacctggtagaaggagcctacctagccctcaacctcaccagtcccgacaaaacccaagagtgctggctgtgtctagtatcgggacccccctactacgaaggggtggccgtcctaggtacttactccaaccatacctctgccccggctaactgctccgtgacctcccaacacaagctgaccctgtccgaagtgaccgggcagggactctgcataggagcagttcccaaaacccatcaggccctgtgtaataccacccagaagacgagcgacgggtcctactatttggcctctcccgccgggaccatttgggcttgcagcaccgggctcactccctgtctatctactactgtgcttaacttaaccactgattactgtgtcctggttgaactctggccaaaggtaacctaccactcccctaattatgtttatggccagtttgaaaagaaaactaaatataaaagagagccggtgtcattaactctggccctgctgttgggaggacttactatgggcggcatagctgcaggagttggaacagggactacagccctagtggccaccaaacaattcgagcagctccaggcagccatacatacagaccttggggccttagaaaaatcagtcagtgccctagaaaagtctctgacctcgttgtctgaggtggtcctacagaaccggaggggattagatctactgttcctaaaagaaggaggattatgtgctgccctaaaagaagaatgctgtttttacgcggaccacactggcgtagtaagagatagcatggcaaagctaagagaaaggttaaaccagagacaaaaattgttcgaatcaggacaagggtggtttgagggactgtttaacaggtccccatggttcacgaccctgatatccaccattatgggccctctgatagtacttttattaatcctactcttcggaccctgtattctcaaccgcttggtccagtttgtaaaagacagaatttcggtagtgcaggccctggttctgacccaacagtatcaccaactcaaatcaatagatccagaagaagtggaatcacgtgaataaaagattttattcagtttccagaaagaggggggaatgaaagaccccaccataaggcttagcacgctagctacagtaacgccattttgcaaggcatggaaaagtaccagagctgagttctcaaaagttacaaggaagtttaattaaagaataaggctgaataacactgggacaggggccaaacaggatatctgtagtcaggcacctgggccccggctcagggccaagaacagatggtcctcagataaagcgaaactaacaacagtttctggaaagtcccacctcagtttcaagttccccaaaagaccgggaaataccccaagccttatttaaactaaccaatcagctcgcttctcgcttctgtacccgcgctttttgctccccagtcctagccctataaaaaaggggtaagaactccacactcggcgcgccagtcatccgatagactgagtcgcccgggtacccgtgttcccaataaagccttttgctgtttgcaa

SEQ ID NO: 2 - DNA, 23 bp, Artificial Sequence
  PCR primer P5588F
gtgtgggtacgccggcaccagac

SEQ ID NO: 3 - DNA, 22 bp, Artificial Sequence
  PCR primer P6304R
tgcatcgaccccccggtgtggc

SEQ ID NO: 4 - DNA, 22 bp, Artificial Sequence
  PCR primer P5641F
ctacaccgtcctgctgacaacc

SEQ ID NO: 5 - DNA, 23 bp, Artificial Sequence
  PCR primer P6171R
tgcctgtccagtggtctcacatc

SEQ ID NO: 6 - protein, 14 aa, Artificial Sequence
  aa 420-433 of XMRV env protein
AALKEECCFYADHT

SEQ ID NO: 7 - DNA, 25 bp, Artificial Sequence
  PCR primer 419F
atcagttaacctacccgagtcggac

SEQ ID NO: 8 - DNA, 23 bp, Artificial Sequence
  PCR primer 1154R
gccgcctcttcttcattgttctc

SEQ ID NO: 9 - DNA, 22 bp, Artificial Sequence
  PCR primer 5922F
gctaatgctacctccctcctgg

SEQ ID NO: 10 - DNA, 26 bp, Artificial Sequence
  PCR primer 6273R
ggagcccactgaggaatcaaaacagg

SEQ ID NO: 11 - DNA, 25 bp, Artificial Sequence
  PCR primer 4F
ccagtcatccgatagactgagtcgc

SEQ ID NO: 12 - DNA, 26 bp, Artificial Sequence
  PCR primer 770R
taccatcctgaggccatcctacattg

SEQ ID NO: 13 - DNA, 23 bp, Artificial Sequence
  PCR primer 350F
gagttcgtattcccggccgcagc

SEQ ID NO: 14 - DNA, 21 bp, Artificial Sequence
  PCR primer 5135R
cctgcggcattccaaatctcg

SEQ ID NO: 15 - DNA, 25 bp, Artificial Sequence
  PCR primer 4789R
gggtgagtctgtgtagggagtctaa

SEQ ID NO: 16 - DNA, 25 bp, Artificial Sequence
  PCR primer 4166F
caagaaggacaacggagagctggag

SEQ ID NO: 17 - DNA, 24 bp, Artificial Sequence
  PCR primer 7622R
ggcctgcactaccgaaattctgtc

SEQ ID NO: 18 - DNA, 27 bp, Artificial Sequence
  PCR primer 4672F
gagccacctacaatcagacaaaaggat

SEQ ID NO: 19 - DNA, 25 bp, Artificial Sequence
  PCR primer 7590R
ctggaccaagcggttgagaatacag

SEQ ID NO: 20 - DNA, 21 bp, Artificial Sequence
  PCR primer 7472F
tcaggacaagggtggtttgag

SEQ ID NO: 21 - DNA, 23 bp, Artificial Sequence
  PCR primer 8182R
caaacagcaaaaggctttattgg

SEQ ID NO: 22 - DNA, 19 bp, Artificial Sequence
  PCR primer 8147R
ccgggcgactcagtctatc

SEQ ID NO: 23 - DNA, 86 bp, Xenotropic murine leukemia virus
ccctgataattatggggatcttggtgagggcaggagcctcagtacaacgtgacagccctcaccaggtctttaatgtcacttggaaa

SEQ ID NO: 24 - DNA, 86 bp, Xenotropic murine leukemia virus; identical to SEQ ID NO: 23

SEQ ID NO: 25 - DNA, 308 bp, Xenotropic murine leukemia virus
ggggccccctgataattatggggatcttggtgagggcaggagcctcagtacaacgtgacagccctcaccaggtctttaatgtcacttggaaaattaccaacctaatgacaggacaaacagctaatgctacctccctcctggggacgatgacagacactttccctaaactatattttgacttgtgtgatttagttggagacaactgggatgacccggaacccgatattggagatggttgccgctctcccgggggaagaaaaaggacaagactatatgatttctatgtttgccccggtcatactgtatta

SEQ ID NO: 26 - DNA, 127 bp, Xenotropic murine leukemia virus
tatggggatcttggtgagggcaggagcctcagtacaacgtgacagccctcaccaggtctttaatgtcacttggaagattaccaacctaatgacaggacaaacagctaatgctacctccctcctgggg

SEQ ID NO: 27 - DNA, 127 bp, Xenotropic murine leukemia virus
tatggggatcttggtgagggcaggagcctcagtacaacgtgacagccctcaccaggtctttaatgtcacttggaaaattaccaacctaatgacaggacaaacagctaatgctacctccctcctgggg

SEQ ID NO: 28 - DNA, 84 bp, Xenotropic murine leukemia virus
tatggggatcttggtgagggcaggagcctcagtacaacgtgacagccctcaccaggtctttaatgtcacttggaaaattaccaa

SEQ ID NO: 29 - DNA, 84 bp, Xenotropic murine leukemia virus; identical to SEQ ID NO: 28

SEQ ID NO: 30 - DNA, 349 bp, Xenotropic murine leukemia virus
gtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgcccaataccctgcccttaccctct

SEQ ID NO: 31 - DNA, 349 bp, Xenotropic murine leukemia virus
gtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccctct

SEQ ID NO: 32 - DNA, 371 bp, Xenotropic murine leukemia virus
ggggccccctgataattatggggatcttggtgagggcaggagcctcagtacaacgtgacagccctcaccaggtctttaatgtcacttggaaaattaccaacctaatgacaggacaaacagctaatgctacctccctcctggggacgatgacagacactttccctaaactatattttgacttgtgtgatttagttggagacaactgggatgacccggaacccgatattggagatggttgccgctctcccgggggaagaaaaaggacaagactatatgatttctatgtttgccccggtcatactgtattaacagggtgtggagggccgagagagggctactgtggcaaatggggatgtgagaccactggacag

SEQ ID NO: 33 - DNA, 140 bp, Xenotropic murine leukemia virus
attcgagcagctccaggcagccatacatacagaccttggggccttagaaaaatcagtcagtgccctagaaaagtctctgacctcgttgtctgaggtggtcctacagaaccggaggggattagatctactgttcctaaaag

SEQ ID NO: 34 - DNA, 140 bp, Xenotropic murine leukemia virus; identical to SEQ ID NO: 33

SEQ ID NO: 35 - DNA, 337 bp, Xenotropic murine leukemia virus
tccagcgcattgcatccaaccagtctgtggatgtcaggaagaggcgctggattaccttctgttccgccgaatggccaactttcaatgtgggatggcctcaggatggtacttttaatttaagtgttatctctcaggtcaagtctagagtgttttgtcctggtccccacggacacccggatcaggtcccatatatcgtcacctgggaggcacttgcctatgacccccctccgtgggtcaaaccgtttgtgtctcctaaacttcctcccttgccgacagctcccgtcctcccgcccggtccttctgcgcaacctccgtcccgatctgccctttaccctgc

SEQ ID NO: 36 - DNA, 337 bp, Xenotropic murine leukemia virus
tccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgc

SEQ ID NO: 37 - DNA, 391 bp, Xenotropic murine leukemia virus
ctgataattatggggatcttggtgagggcaggagcctcagtacaacgtgacagccctcaccaggtctttaatgtcacttggaaaattaccaacctaatgacaggacaaacagctaatgctacctccctcctggggacgatgacagacactttccctaaactatattttgacttgtgtgatttagttggagacaactgggatgacccggaacccgatattggagatggttgccgctctcccgggggaagaaaaaggacaagactatatgatttctatgtttgccccggtcatactgtattaacagggtgtggagggccgagagagggctactgtggcaaatggggatgtgagaccactggacaggcatactggaagccatcatcatcatggg

SEQ ID NO: 38 - DNA, 205 bp, Xenotropic murine leukemia virus
ggatggtacttttaatttaggtgttatctctcaggtcaagtctagagtgttttgtcctggtccccatcgacacccggatcaggtcccatatatcgtcacctgggaggcacttgcctatgacccccctccgtgggtcaaaccgtttgtctctcctaaaccccctcctttaccgacagctcccgtcctcccgcccggtccttctgcg

SEQ ID NO: 39 - DNA, 205 bp, Xenotropic murine leukemia virus
ggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcg

SEQ ID NO: 40 - DNA, 334 bp, Xenotropic murine leukemia virus
gcatccaaccagtctgtggatgtcaagaagagggctgggttaccttctgttccgccgaatggccaactttcaatgtaggatggcctcaggatggtacttttaatttaggtgttatctctcaggtcaagtctagagtgttttgtcctggtccccactgacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgaaacctccgtcccgatctgccctttaccctgcccttaccct

SEQ ID NO: 41 - DNA, 335 bp, Xenotropic murine leukemia virus
gcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccct

SEQ ID NO: 42 - protein, 208 aa, Spleen focus-forming virus
VQLDSPHQVSNVTWRVTNLMTGQTANATSLLGTMTEAFPKLYFDLCDLMGDDWDETGLGCRTPGGRKRARTFDFYVCPGHTVPTGCGGPREGYCGKWGCETTGQAYWKPSSSWDLISLKRGNTPKDQGPCYDSSVSSGVLGATPGGRCNPLVLEFTDAGRKASWDAPKVWGRLYRSTGTDPVTRFSLTRQVLDIGPRVPIGSNPVTTD

SEQ ID NO: 43 - protein, 214 aa, Xenotropic murine leukemia virus
VQRDSPHQVFNVTWKITNLMTGQTANATSLLGTMTDTFPKLYFDLCDLVGDHWDDPEPDIGDGCRSPGGRKRTRLYDFYVCPGHTVLTGCGGPREGYCGKWGCETTGQAYWKPSSSWDLISLKRGNTPKGQGPCFDSSVGSGSIQGATPGGRCNPLVLEFTDAGKRASWDAPKTWGLRLYRSTGADPVTLFSLTRQVLNVGPRVPIGPNPVITE

SEQ ID NO: 44 - DNA, 490 bp, Xenotropic murine leukemia virus
acccgtggggccccctgataattatggggatcttggtgagggcaggagcctcagtacaacgtgacagccctcaccaggtctttaatgtcacttggaaaattaccaacctaatgacaggacaaacagctaatgctacctccctcctggggacgatgacagacactttccctaaactatattttgacttgtgtgatttagttggagacaactgggatgacccggaacccgatattggagatggttgccgctctcccgggggaagaaaaaggacaagactatatgatttctatgtttgccccggtcatactgtattaacagggtgtggagggccgagagagggctactgtggcaaatggggatgtgagaccactggacaggcatactggaagccatcatcatcatgggacctaatttcccttaagcgaggaaacactcctaagggtcagggcccctgttttgattcctcagtgggctccggtagcatccaggg

SEQ ID NO: 45 - DNA, 391 bp, Xenotropic murine leukemia virus
ctgataattatggggatcttggtgagggcaggagcctcagtacaacgtgacagccctcaccaggtctttaatgtcacttggaaaattaccaacctaatgacaggacaaacagctaatgctacctccctcctggggacgatgacagacactttccctaaactatattttgacttgtgtgatttagttggagacaactgggatgacccggaacccgatattggagatggttgccgctctcccgggggaagaaaaaggacaagactatatgatttctatgtttgccccggtcatactgtattaacagggtgtggagggccgagagagggctactgtggcaaatggtgatgtgagaccactggacaggcatactggaagccatcatcatcatggg

SEQ ID NO: 46 - DNA, 489 bp, Xenotropic murine leukemia virus
acccgtggggcccctgataattatggggatcttggtgagggcaggagcctcagtacaacgtgacagccctcaccaggtctttaatgtcacttggaaaattaccaacctaatgacaggacaaacagctaatgctacctccctcctggggacgatgacagacactttccctaaactatattttgacttgtgtgatttagttggagacaactgggatgacccggaacccgatattggagatggttgccgctctcccgggggaagaaaaaggacaagactatatgatttctatgtttgccccggtcatactgtattaacagggtgtggagggccgagagagggctactgtggcaaatggggatgtgagaccactggacaggcatactggaagccatcatcatcatgggacctaatttcccttaagcgaggaaacactcctaagggtcagggcccctgttttgattcctcagtgggctccggtagcatccaggg

SEQ ID NO: 47 - DNA, 291 bp, Xenotropic murine leukemia virus
taattatggggatcttggtgagggcaggagcctcagtacaacgtgacagccctcaccaggtctttaatgtcacttggaaaattaccaacctaatgacaggacaaacagctaatgctacctccctcctggggacgatgacagacactttccctaaactatattttgacttgtgtgatttagttggagacaactgggatgacccggaacccgatattggagatggttgccgctctcccgggggaagaaaaaggacaagactatatgatttctatgtttgccccggtcatactg

SEQ ID NO: 48 - DNA, 370 bp, Xenotropic murine leukemia virus
ggggcccctgataattatggggatcttggtgagggcaggagcctcagtacaacgtgacagccctcaccaggtctttaatgtcacttggaaaattaccaacctaatgacaggacaaacagctaatgctacctccctcctggggacgatgacagacactttccctaaactatattttgacttgtgtgatttagttggagacaactgggatgacccggaacccgatattggagatggttgccgctctcccgggggaagaaaaaggacaagactatatgatttctatgtttgccccggtcatactgtattaacagggtgtggagggccgagagagggctactgtggcaaatggggatgtgagaccactggacag

SEQ ID NO: 49 - DNA, 307 bp, Xenotropic murine leukemia virus
ggggcccctgataattatggggatcttggtgagggcaggagcctcagtacaacgtgacagccctcaccaggtctttaatgtcacttggaaaattaccaacctaatgacaggacaaacagctaatgctacctccctcctggggacgatgacagacactttccctaaactatattttgacttgtgtgatttagttggagacaactgggatgacccggaacccgatattggagatggttgccgctctcccgggggaagaaaaaggacaagactatatgatttctatgtttgccccggtcttactgtatta

SEQ ID NO: 50 - protein, 161 aa, Xenotropic murine leukemia virus
PWGPLIIMGILVRAGASVQRDSPHQVFNVTWKITNLMTGQTANATSLLGTMTDTFPKLYFDLCDLVGDNWDDPEPDIGDGCRSPGGRKRTRLYDFYVCPGHTVLTGCGGPREGYGKWGCETTGQAYWKPSSSWDLISLKRGNTPKGQGPCFDSSVGSGSIQ

SEQ ID NO: 51 - protein, 130 aa, Xenotropic murine leukemia virus
  MISC_FEATURE (115)..(115): stop
LIIMGILVRAGASVQRDSPHQVFNVTWKITNLMTGQTANATSLLGTMTDTFPKLYFDLCDLVGDNWDDPEPDIGDGCRSPGGRKRTRLYDFYVCPGHTVLTGCGGPREGYCGKWXCETTGQAYWKPSSSW

SEQ ID NO: 52 - protein, 162 aa, Xenotropic murine leukemia virus
PVGPLIIMGILVRAGASVQRDSPHQVFNVTWKITNLMTGQTANATSLLGTMTDTFPKLYFDLCDLVGDNWDDPEPDIGDGCRSPGGRKRTRLYDFYVCPGHTVLTGCGGPREGYCGKWGCETTGQAYWKPSSSWDLISLKRGNTPKGQGPCFDSSVGSGSIQ

SEQ ID NO: 53 - protein, 96 aa, Xenotropic murine leukemia virus
IMGILVRAGASVQRDSPHQVFNVTWKITNLMTGQTANATSLLGTMTDTFPKLYFDLCDLVGDNWDDPEPDIGDGCRSPGGRKRTRLYDFYVCPGHT

SEQ ID NO: 54 - protein, 123 aa, Xenotropic murine leukemia virus
GPLIIMGILVRAGASVQRDSPHQVFNVTWKITNLMTGQTANATSLLGTMTDTFPKLYFDLCDLVGDNWDDPEPDIGDGCRSPGGRKRTRLYDFYVCPGHTVLTGCGGPREGYCGKWGCETTGQ

SEQ ID NO: 55 - protein, 102 aa, Xenotropic murine leukemia virus
GPLIIMGILVRAGASVQRDSPHQVFNVTWKITNLMTGQTANATSLLGTMTDTFPKLYFDLCDLVGDNWDDPEPDIGDGCRSPGGRKRTRLYDFYVCPGLTVL

SEQ ID NO: 56 - DNA, 322 bp, Xenotropic murine leukemia virus
caattcgagcagctccaggcagccatacatacagaccttggggccttagaaaaatcagtcagtgccctagaaaagtctctgacctcgttgtctgaggtggtcctacagaaccggaggggattagatctactgttcctaaaagaaggaggattatgtgctgccctaaaagaagaatgctgtttttacgcggaccacactggcgtagtaagagatagcatggcaaagctaagagaaaggttaaaccagagacaaaaattgttcgaatcacgacaagggtggtttgagggactgtttaacaggtccccatggttcacgaccctga

SEQ ID NO: 57 - DNA, 143 bp, Xenotropic murine leukemia virus
ttcgagcagctccaggcagccatacatacagaccttggggccttagaaaaatcagtcagtgccctagaaaagtctctgacctcgttgtctgaggtggtcctacagaaccggaggggattagatctactgttcctaaaagaagg

SEQ ID NO: 58 - DNA, 314 bp, Xenotropic murine leukemia virus
ttcgagcagctccaggcagccatacatacagaccttggggccttagaaaaatcagtcagtgccctagaaaagtctctgacctcgttgtctgaggtggtcctacagaaccggaggggattagatctactgttcctaaaagaaggaggattatgtgctgccctaaaagaagaatgctgtttttacgcggaccacactggcgtagtaagagctagcatggcaaagctaagagaaaggttaaaccagagacaaaaattgttcgaatcaggacaagggtggtttgagggactgtttaacaggtccccatggttcacgac

SEQ ID NO: 59 - DNA, 310 bp, Xenotropic murine leukemia virus
agcagctccaggcagccatacatacagaccttggggccttagaaaaatcagtcagtgccctagaaaagtctctgacctcgttgtctgaggtggtcctacagaaccggaggggattagatctactgttcctaaaagaaggaggattatgtgctgccctaaaagaagaatgctgtttttacgcggaccacactggcgtagtaagagatagcatggcaaagctaagagaaaggttaaaccagagacaaaaattgttcgaatcaggacaagggtggtttgagggactgtttaacaggtccccatggttcacgac

SEQ ID NO: 60 - DNA, 370 bp, Xenotropic murine leukemia virus
agcagctccaggcagccatacatacagaccttggggccttagaaaaatcagtcagtgccctagaaaagtctctgacctcgttgtctgaggtggtcctacagaaccggaggggattagatctagaaaagtctctgacctcgttgtctgaggtggtcctacagaaccggaggggattagatctactgttcctaaaagaaggaggattatgtgctgccctaaaagaagaatgctgtttttacgcggaccacactggcgtagtaagagatagcatggcaaagctaagagaaaggttaaaccagagacaaaaattgttcgaatcaggacaagggtggtttgagggactgtttaacaggtccccatggttcacgac

SEQ ID NO: 61 - DNA, 314 bp, Xenotropic murine leukemia virus
caattcgagcagctccaggcagccatacatacagaccttggggccttagaaaaatcagtcagtgccctagaaaagtctctgacctcgttgtctgaggtggtcctacagaaccggaggggattagatctactgttcctaaaagaaggaggattatgtgctgccctaaaagaagaatgctgtttttacgcggaccacactggcgtagtaagagatagcatggcaaagctaagagaaaggttaaaccagagacaaaaattgttcgaatcaggacaagggtggtttgagggactgtttaacaggtccccatggttcac

SEQ ID NO: 62 - DNA, 309 bp, Xenotropic murine leukemia virus
attcgagcagctccaggcagccatacatacagaccttggggccttagaaaaatcagtcagtgccctagaaaagtctctgacctcgttgtctgaggtggtcctacagaaccggaggggattagatctactgttcctaaaagaaggaggattatgtgctgccctaaaagaagaatgctgtttttacgcggaccacactggcgtagtaagagatagcatggcaaagctaagagaaaggttaaaccagagacaaaaattgttcgaatcaggacaagggtggtttgagggactgtttaacaggtccccatggtt

SEQ ID NO: 63 - DNA, 322 bp, Xenotropic murine leukemia virus
ccaaacaattcgagcagctccaggcagccatacatacagaccttggggccttagaaaaatcagtcagtgccctagaaaagtctctgacctcgttgtctgaggtggtcctacagaaccggaggggattagatctactgttcctaaaagaaggaggattatgtgctgccctaaaagaagaatgctgtttttacgcggaccacactggcgtagtaagagatagcatggcaaagctaagagaaaggttaaaccagagacaaaaattgttcgaatcaggacaagggtggtttgagggactgtttaacaggtccccatggttcacgac

SEQ ID NO: 64 - DNA, 319 bp, Xenotropic murine leukemia virus
acaattcgagcagctccaggcagccatacatacagaccttggggccttagaaaaatcagtcagtgccctagaaaagtctctgacctcgttgtctgaggtggtcctacagaaccggaggggattagatctactgttcctaaaagaaggaggattatgtgctgccctaaaagaagaatgctgtttttacgcggaccacactggcgtagtaagagatagcatggcaaagctaagagaaaggttaaaccagagacaaaaattgttcgaatcaggacaagggtggtttgagggactgtttaacaggtccccatggttcacgacc

SEQ ID NO: 65 - protein, 107 aa, Xenotropic murine leukemia virus
QFEQLQAAIHTDLGALEKSVSALEKSLTSLSEVVLQNRRGLDLLFLKEGGLCAALKEECCFYADHTGVVRDSMAKLRERLNQRQKLFESRQGWFEGLFNRSPWFTTL

SEQ ID NO: 66 - protein, 47 aa, Xenotropic murine leukemia virus
FEQLQAAIHTDLGALEKSVSALEKSLTSLSEVVLQNRRGLDLLFLKE

SEQ ID NO: 67 - protein, 104 aa, Xenotropic murine leukemia virus
FEQLQAAIHTDLGALEKSVSALEKSLTSLSEVVLQNRRGLDLLFLKEGGLCAALKEECCFYADHTGVVRASMAKLRERLNQRQKLFESGQGWFEGLFNRSPWFT

SEQ ID NO: 68 - protein, 102 aa, Xenotropic murine leukemia virus
QLQAAIHTDLGALEKSVSALEKSLTSLSEVVLQNRRGLDLLFLKEGGLCAALKEECCFYADHTGVVRDSMAKLRERLNQRQKLFESGQGWFEGLFNRSPWFT

SEQ ID NO: 69 - protein, 122 aa, Xenotropic murine leukemia virus
QLQAAIHTDLGALEKSVSALEKSLTSLSEVVLQNRRGLDLEKSLTSLSEVVLQNRRGLDLLFLKEGGLCAALKEECCFYADHTGVVRDSMAKLRERLNQRQKLFESGQGWFEGLFNRSPWFT

SEQ ID NO: 70 - protein, 104 aa, Xenotropic murine leukemia virus
QFEQLQAAIHTDLGALEKSVSALEKSLTSLSEVVLQNRRGLDLLFLKEGGLCAALKEECCFYADHTGVVRDSMAKLRERLNQRQKLFESGQGWFEGLFNRSPWF

SEQ ID NO: 71 - protein, 102 aa, Xenotropic murine leukemia virus
FEQLQAAIHTDLGALEKSVSALEKSLTSLSEVVLQNRRGLDLLFLKEGGLCAALKEECCFYADHTGVVRDSMAKLRERLNQRQKLFESGQGWFEGLFNRSPW

SEQ ID NO: 72 - protein, 106 aa, Xenotropic murine leukemia virus
KQFEQLQAAIHTDLGALEKSVSALEKSLTSLSEVVLQNRRGLDLLFLKEGGLCAALKEECCFYADHTGVVRDSMAKLRERLNQRQKLFESGQGWFEGLFNRSPWFT

SEQ ID NO: 73 - protein, 106 aa, Xenotropic murine leukemia virus
QFEQLQAAIHTDLGALEKSVSALEKSLTSLSEVVLQNRRGLDLLFLKEGGLCAALKEECCFYADHTGVVRDSMAKLRERLNQRQKLFESGQGWFEGLFNRSPWFTT

SEQ ID NO: 74 - DNA, 354 bp, Xenotropic murine leukemia virus
atgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccccctcta

SEQ ID NO: 75 - DNA, 334 bp, Xenotropic murine leukemia virus
gtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttacc

SEQ ID NO: 76 - DNA, 333 bp, Xenotropic murine leukemia virus
gcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctga

SEQ ID NO: 77 - DNA, 338 bp, Xenotropic murine leukemia virus
gtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctga

SEQ ID NO: 78 - DNA, 339 bp, Xenotropic murine leukemia virus
tgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgc

SEQ ID NO: 79 - DNA, 331 bp, Xenotropic murine leukemia virus
gtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgcccttt

SEQ ID NO: 80 - DNA, 325 bp, Xenotropic murine leukemia virus
gtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccgggccttctgcgcaacctccgtcccgatctg

SEQ ID NO: 81 - DNA, 336 bp, Xenotropic murine leukemia virus
gtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccct

SEQ ID NO: 82 - DNA, 346 bp, Xenotropic murine leukemia virus
atgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttac

SEQ ID NO: 83 - DNA, 335 bp, Xenotropic murine leukemia virus
tgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaca

SEQ ID NO: 84 - DNA, 340 bp, Xenotropic murine leukemia virus
tgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcc

SEQ ID NO: 85 - DNA, 344 bp, Xenotropic murine leukemia virus
tccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttacc

SEQ ID NO: 86 - DNA, 349 bp, Xenotropic murine leukemia virus
tgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcctttaccctc

SEQ ID NO: 87 - DNA, 348 bp, Xenotropic murine leukemia virus
atgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccc

SEQ ID NO: 88 - DNA, 343 bp, Xenotropic murine leukemia virus
tgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcctat

SEQ ID NO: 89 - DNA, 350 bp, Xenotropic murine leukemia virus
tgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccctca

SEQ ID NO: 90 - DNA, 343 bp, Xenotropic murine leukemia virus
ccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttacc

SEQ ID NO: 91 - DNA, 325 bp, Xenotropic murine leukemia virus
ttgcatccaaccagtctgtggatgtcaagaagaggcgctgggttaccttctgttccgccgaatggccaactttcaatgtaggatggcctcaggatggtacttttaatttaggtattatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccc

SEQ ID NO: 92 - DNA, 345 bp, Xenotropic murine leukemia virus
ccagcgcattgcatccaaccagtctgtggatgtcaagaagaggcgctgggtaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccctc

SEQ ID NO: 93 - DNA, 329 bp, Xenotropic murine leukemia virus
gtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccca

SEQ ID NO: 94 - DNA, 340 bp, Xenotropic murine leukemia virus
tgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacgggcacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcc

SEQ ID NO: 95 - DNA, 350 bp, Xenotropic murine leukemia virus
tgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcctttaccctct

SEQ ID NO: 96 - DNA, 346 bp, Xenotropic murine leukemia virus
cagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccctct

SEQ ID NO: 97 - DNA, 340 bp, Xenotropic murine leukemia virus
tccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgccta

SEQ ID NO: 98 - DNA, 329 bp, Xenotropic murine leukemia virus
gtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccct

SEQ ID NO: 99 - DNA, 349 bp, Xenotropic murine leukemia virus
tgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccctc

SEQ ID NO: 100 - DNA, 329 bp, Xenotropic murine leukemia virus; identical to SEQ ID NO: 98

SEQ ID NO: 101 - DNA, 351 bp, Xenotropic murine leukemia virus
atgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccctct

SEQ ID NO: 102 - DNA, 350 bp, Xenotropic murine leukemia virus
tgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccctct

SEQ ID NO: 103 - DNA, 347 bp, Xenotropic murine leukemia virus
gtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccct

SEQ ID NO: 104 - DNA, 332 bp, Xenotropic murine leukemia virus
atgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctt

SEQ ID NO: 105 - DNA, 329 bp, Xenotropic murine leukemia virus
tgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaatctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccca

SEQ ID NO: 106 - DNA, 316 bp, Xenotropic murine leukemia virus
tggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttac

SEQ ID NO: 107 - DNA, 334 bp, Xenotropic murine leukemia virus
gcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccc

SEQ ID NO: 108 - DNA, 340 bp, Xenotropic murine leukemia virus
tccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgccct

SEQ ID NO: 109 - DNA, 343 bp, Xenotropic murine leukemia virus
tccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttac

SEQ ID NO: 110 - DNA, 344 bp, Xenotropic murine leukemia virus
tgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgccctta

SEQ ID NO: 111 - DNA, 346 bp, Xenotropic murine leukemia virus
tccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccct

SEQ ID NO: 112 - DNA, 348 bp, Xenotropic murine leukemia virus; identical to SEQ ID NO: 87

SEQ ID NO: 113 - DNA, 346 bp, Xenotropic murine leukemia virus
gtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccc

SEQ ID NO: 114 - DNA, 343 bp, Xenotropic murine leukemia virus
gtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgccctta

SEQ ID NO: 115 - protein, 117 aa, Xenotropic murine leukemia virus
VQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALTPS

SEQ ID NO: 116 - protein, 111 aa, Xenotropic murine leukemia virus
VQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALY

SEQ ID NO: 117 - protein, 110 aa, Xenotropic murine leukemia virus
RIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYP

SEQ ID NO: 118 - protein, 112 aa, Xenotropic murine leukemia virus
VQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYP

SEQ ID NO: 119 - protein, 112 aa, Xenotropic murine leukemia virus; identical to SEQ ID NO: 118

SEQ ID NO: 120 - protein, 110 aa, Xenotropic murine leukemia virus
VQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSAL

SEQ ID NO: 121 - protein, 108 aa, Xenotropic murine leukemia virus
VQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRS

SEQ ID NO: 122 - protein, 112 aa, Xenotropic murine leukemia virus; identical to SEQ ID NO: 118

SEQ ID NO: 123 - protein, 114 aa, Xenotropic murine leukemia virus
VQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPAL

SEQ ID NO: 124 - protein, 111 aa, Xenotropic murine leukemia virus; identical to SEQ ID NO: 116

SEQ ID NO: 125 - protein, 113 aa, Xenotropic murine leukemia virus
VQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPA

SEQ ID NO: 126 - protein, 114 aa, Xenotropic murine leukemia virus
QRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALT

SEQ ID NO: 127 - protein, 116 aa, Xenotropic murine leukemia virus
VQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPAFTL

SEQ ID NO: 128 - protein, 115 aa, Xenotropic murine leukemia virus
VQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALT

SEQ ID NO: 129 - protein, 114 aa, Xenotropic murine leukemia virus
VQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPAY

SEQ ID NO: 130 - protein, 116 aa, Xenotropic murine leukemia virus
VQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALTL

SEQ ID NO: 131 - protein, 114 aa, Xenotropic murine leukemia virus; identical to SEQ ID NO: 126

SEQ ID NO: 132 - protein, 107 aa, Xenotropic murine leukemia virus
ASNQSVDVKKRRWVTFCSAEWPTFNVGWPQDGTFNLGIISQVKSRVFCPGPHGHPDQVPYIVTWEALAYDPPPWVKPFVSPKPPPLPTAPVLPPGPSAQPPSRSALY

SEQ ID NO: 133 - protein, 112 aa, Xenotropic murine leukemia virus
QRIASNQSVDVKKRRWVPSVPPNGQLSMDGLRMVLLIVLSLRSSLECFVLVPTDTRIRSHISSPGRHLPMTPLRGSNRLSLLNPLLYRQLPSSRPVLLRNLRPDLPFTLPLP

SEQ ID NO: 134 - protein, 109 aa, Xenotropic murine leukemia virus
VQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSA

SEQ ID NO: 135 - protein, 113 aa, Xenotropic murine leukemia virus; identical to SEQ ID NO: 125

SEQ ID NO: 136 - protein, 116 aa, Xenotropic murine leukemia virus; identical to SEQ ID NO: 127

SEQ ID NO: 137 - protein, 115 aa, Xenotropic murine leukemia virus
QRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALTL

SEQ ID NO: 138 - protein, 112 aa, Xenotropic murine leukemia virus
QRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPA

SEQ ID NO: 139 - protein, 109 aa, Xenotropic murine leukemia virus; identical to SEQ ID NO: 134

SEQ ID NO: 140 - protein, 116 aa, Xenotropic murine leukemia virus; identical to SEQ ID NO: 130

SEQ ID NO: 141 - protein, 109 aa, Xenotropic murine leukemia virus; identical to SEQ ID NO: 134

SEQ ID NO: 142 - protein, 116 aa, Xenotropic murine leukemia virus; identical to SEQ ID NO: 130

SEQ ID NO: 143 - protein, 116 aa, Xenotropic murine leukemia virus; identical to SEQ ID NO: 130

SEQ ID NO: 144 - protein, 115 aa, Xenotropic murine leukemia virus; identical to SEQ ID NO: 128

SEQ ID NO: 145 - protein, 110 aa, Xenotropic murine leukemia virus; identical to SEQ ID NO: 120

SEQ ID NO: 146 - protein, 109 aa, Xenotropic murine leukemia virus
VQRIASNQSVDVKKRRWVTFCSAEWPTFNVGWPQDGTFNLGVISQVNLECFVLVPTDTRIRSHISSPGRHLPMTPLRGSNRLSLLNPLLYRQLPSSRPVLLRNLRPDLP

SEQ ID NO: 147 - protein, 104 aa, Xenotropic murine leukemia virus
DVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPAL

SEQ ID NO: 148 - protein, 111 aa, Xenotropic murine leukemia virus
ASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALT

SEQ ID NO: 149 - protein, 112 aa, Xenotropic murine leukemia virus; identical to SEQ ID NO: 138

SEQ ID NO: 150 - protein, 113 aa, Xenotropic murine leukemia virus
QRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPAL

SEQ ID NO: 151 - protein, 114 aa, Xenotropic murine leukemia virus; identical to SEQ ID NO: 123

SEQ ID NO: 152 - protein, 114 aa, Xenotropic murine leukemia virus; identical to SEQ ID NO: 126

SEQ ID NO: 153 - protein, 115 aa, Xenotropic murine leukemia virus; identical to SEQ ID NO: 128

SEQ ID NO: 154 - protein, 115 aa, Xenotropic murine leukemia virus; identical to SEQ ID NO: 128

SEQ ID NO: 155 - protein, 114 aa, Xenotropic murine leukemia virus; identical to SEQ ID NO: 123

SEQ ID NO: 156 - DNA, 20 bp, Artificial Sequence
  PCR primer
gcagggtccccaacaactgc

SEQ ID NO: 157 - DNA, 20 bp, Artificial Sequence
  PCR primer
gggagacgtccagcgcattg

SEQ ID NO: 158 - DNA, 20 bp, Artificial Sequence
  PCR primer
ctttcgatgtggggtggccg

SEQ ID NO: 159 - DNA, 20 bp, Artificial Sequence
  PCR primer
cccatgcggagtgggaatgc

SEQ ID NO: 160 - protein, 645 aa, Xenotropic murine leukemia virus
MESPAFSKPLKDKINPWGPLIIMGILVRAGASVQRDSPHQVFNVTWKITNLMTGQTANATSLLGTMTDTFPKLYFDLCDLVGDNWDDPEPDIGDGCRSPGGRKRTRLYDFYVCPGHTVLTGCGGPREGYCGKWGCETTGQAYWKPSSSWDLISLKRGNTPKGQGPCFDSSVGSGSIQGATPGGRCNPLVLEFTDAGKRASWDAPKTWGLRLYRSTGADPVTLFSLTRQVLNVGPRVPIGPNPVITEQLPPSQPVQIMLPRTPRPPPSGAASMVPGAPPPSQQPGTGDRLLNLVEGAYLALNLTSPDKTQECWLCLVSGPPYYEGVAVLGTYSNHTSAPANCSVTSQHKLTLSEVTGQGLCIGAVPKTHQALCNTTQKTSDGSYYLASPAGTIWACSTGLTPCLSTTVLNLTTDYCVLVELWPKVTYHSPNYVYGQFEKKTKYKREPVSLTLALLLGGLTMGGIAAGVGTGTTALVATKQFEQLQAAIHTDLGALEKSVSALEKSLTSLSEVVLQNRRGLDLLFLKEGGLCAALKEECCFYADHTGVVRDSMAKLRERLNQRQKLFESGQGWFEGLFNRSPWFTTLISTIMGPLIVLLLILLFGPCILNRLVQFVKDRISVVQALVLTQQYHQLKSIDPEEVESRE

SEQ ID NO: 161 - protein, 1733 aa, Xenotropic murine leukemia virus
  misc_feature (537)..(537): Xaa can be any naturally occurring amino acid
MGQTVTTPLSLTLQHWGDVQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALTPSIKSKPPKPQVLPDSGGPLIDLLTEDPPPYGAQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEARKAVRGNDGRPTQLPNEVNAAFPLERPDWDYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGDXGGQGQEPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVVGPMGQPLQVLTLNIEDEYRLHETSKEPDVPLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSEQDCQRGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQAMLLDTDRVQFGPVVALNPATLLPLPEKEAPHDCLEILAETHGTRPDLTDQPIPDADYTWYTDGSSFLQEGQRRAGAAVTTETEVIWARALPAGTSAQRAELIALTQALKMAEGKKLNVYTDSRYAFATAHVHGEIYRRRGLLTSEGREIKNKNEILALLKALFLPKRLSIIHCPGHQKGNSAEARGNRMADQAAREAAMKAVLETSTLLIEDSTPYTPPHFHYTETDLKRLRELGATYNQTKGYWVLQGKPVMPDQSVFELLDSLHRLTHLSPQKMKALLDREESPYYMLNRDRTIQYVTETCTACAQVNASKAKIGAGVRVRGHRPGTHWEVDFTEVKPGLYGYKYLLVFVDTFSGWVEAFPTKRETAKVVSKKLLEDIFPRFGMPQVLGSDNGPAFASQVSQSVADLLGIDWKLHCAYRPQSSGQVERMNRTIKETLTKLTLASGTRDWVLLLPLALYRARNTPGPHGLTPYEILYGAPPPLVNFHDPEMSKLTNSPSLQAHLQALQAVQQEVWKPLAAAYQDQLDQPVIPHPFRVGDAVWVRRHQTKNLEPRWKGPYTVLLTTPTALKVDGISAWIHAAHVKAATTPPAGTAWKVQRSQNPLKIRLTRGAP

SEQ ID NO: 162 - DNA, 8165 bp, Xenotropic murine leukemia virus
agtcatccgatagactgagtcgcccgggtacccgtgttcccaataaagccttttgctgtttgcatccgaagcgtggcctcgctgttccttgggagggtctcctcagagtgattgactacccagctcgggggtctttcatttgggggctcgtccgggattcggagacccccgcccagggaccaccgacccaccgtcgggaggtaagccggccggcgatcgttttgtctttgtctctgtctttgtgcgtgtgtgtgtgtgccggcatctaatcctcgcgcctgcgtctgaatctgtactagttagctaactagatctgtatctggcggttccgcggaagaactgacgagttcgtattcccggccgcagccctgggagacgtcccagcggcctcgggggcccgttttgtggcccattctgtatcagttaacctacccgagtcggactttttggagtggctttgttgggggacgagagacagagacacttcccgcccccgtctgaatttttgctttcggttttacgccgaaaccgcgccgcgcgtctgatttgttttgttgttcttctgttcttcgttagttttcttctgtctttaagtgttctcgagatcatgggacagaccgtaactacccctctgagtctaaccttgcagcactggggagatgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccccctctataaagtccaaacctcctaagccccaggttctccctgatagcggcggacctctcattgaccttctcacagaggatcccccgccgtacggagcacaaccttcctcctctgccagggagaacaatgaagaagaggcggccaccacctccgaggtttcccccccttctcccatggtgtctcgactgcggggaaggagagaccctcccgcagcggactccaccacctcccaggcattcccactccgcatggggggagatggccagcttcagtactggccgttttcctcctctgatttatataattggaaaaataataacccttccttttctgaagatccaggtaaattgacggccttgattgagtccgtcctcatcacccaccagcccacctgggacgactgtcagcagttgttggggaccctgctgaccggagaagaaaagcagcgggtgctcctagaggctagaaaggcagtccggggcaatgatggacgccccactcagttgcctaatgaagtcaatgctgcttttccccttgagcgccccgattgggattacaccactacagaaggtaggaaccacctagtcctctaccgccagttgctcttagcgggtctccaaaacgcgggcaggagccccaccaatttggccaaggtaaaagggataacccagggacctaatgagtctccctcagcctttttagagagactcaaggaggcctatcgcaggtacactccttatgaccctgaggacccagggcaagaaaccaatgtgtccatgtcattcatctggcagtctgccccggatatcggacgaaagttagagcggttagaagatttaaagagcaagaccttaggagacttagtgagggaagctgaaaagatctttaataagcgagaaaccccggaagaaagagaggaacgtatcaggagagaaatagaggaaaaagaagaacgccgtagggcagaggatgagcagagagagagagaaagggaccgcagaagacatagagagatgagcaagctcttggccactgtagttattggtcagagacaggatagacaggggggagagcggaggaggccccaacttgataaggaccaatgcgcctactgcaaagaaaagggacactgggctaaggactgcccaaagaagccacgagggccccgaggaccgaggccccagacctccctcctgaccttaggtgactagggaggtcagggtcaggagcccccccctgaacccaggataaccctcaaagtcggggggcaacccgtcaccttcctggtagatactggggcccaacactccgtgctgacccaaaatcctggacccctaagtgacaagtctgcctgggtccaaggggctactggaggaaagcggtatcgctggaccacggatcgcaaagtacatctggctaccggtaaggtcacccactctttcctccatgtaccagactgcccctatcctctgctaggaagagacttgctgactaaactaaaagcccaaatccacttcgagggatcaggagctcaggttgtgggaccgatgggacagcccctgcaagtgctgaccctaaacatagaagatgagtatcggctacatgagacctcaaaagagccagatgttcctctagggtccacatggctttctgattttccccaggcctgggcggaaaccgggggcatgggactggcagttcgccaagctcctctgatcatacctctgaaggcaacctctacccccgtgtccataaaacaataccccatgtcacaagaagccagactggggatcaagccccacatacagaggctgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaactgacctggaccagactcccacagggtttcaaaaacagtcccaccctgtttgatgaggcactgcacagagacctagcagatttccggatccagcacccagacttgatcctgctacagtacgtggatgacttactgctggccgccacttctgagcaagactgccaacgaggtactcgggccctattacaaaccctagggaacctcgggtatcgggcctcggccaagaaagcccaaatttgccagaaacaggtcaagtatctggggtatctcctaaaagagggacagagatggctgactgaggccagaaaagagactgtgatggggcagcccactccgaagacccctcgacaactaagggagttcctagggacggcaggcttctgtcgcctctggatccctgggtttgcagaaatggcagcccccttgtaccctcttaccaaaacggggactctgtttaattggggcccagaccagcaaaaggcctatcaagaaatcaaacaggctcttctaactgcccccgccctgggattgccagatttgactaagccctttgaactctttgtcgacgagaagcagggctacgccaaaggcgtcctaacgcaaaaactgggaccttggcgtcggcctgtggcctacctgtccaaaaagctagacccagtggcagctgggtggcccccttgcctacggatggtagcagccattgccgttctgacaaaggatgcaggcaagctaactatgggacagccgctagtcattctggccccccatgcggtagaagcactggtcaaacaaccccctgaccgttggctatccaatgcccgcatgacccactatcaggcaatgctcctggatacagaccgggttcagttcggaccggtggtggccctcaacccggccaccctgctccccctaccggaaaaggaagccccccatgactgcctcgagatcttggctgagacgcacggaaccagaccggacctcacggaccagcccatcccagacgctgattacacttggtacacagatggaagcagcttcctacaagaaggacaacggagagctggagcagcggtgactactgagaccgaggtaatctgggcgagggctctgccggctggaacatccgcccaacgagccgaactgatagcactcacccaagccttaaagatggcagaaggtaagaagctaaatgtttacactgatagccgctatgccttcgccacggcccatgtccatggagaaatatataggaggcgagggttgctgacctcagaaggcagagaaattaaaaacaagaacgagatcttggccttgctaaaagctctctttctgcccaaacgacttagtataattcactgtccaggacatcaaaaaggaaacagtgctgaggccagaggcaaccgtatggcagatcaagcagcccgagaggcagccatgaaggcagttctagaaacctctacactcctcatagaggactcaaccccgtatacgcctccccatttccattacaccgaaacagatctcaaaagactacgggaactgggagccacctacaatcagacaaaaggatattgggtcctacaaggcaaacctgtgatgcccgatcagtccgtgtttgaactgttagactccctacacagactcacccatctgagccctcaaaagatgaaggcactcctcgacagagaagaaagcccctactacatgttaaaccgggacagaactatccagtatgtgactgagacctgcaccgcctgtgcccaagtaaatgccagcaaagccaaaattggggcaggggtgcgagtacgcggacatcggccaggcacccattgggaagttgatttcacggaagtaaagccaggactgtatgggtacaagtacctcctagtgtttgtagacaccttctctggctgggtagaggcattcccgaccaagcgggaaactgccaaggtcgtgtccaaaaagctgttagaagacatttttccgagatttggaatgccgcaggtattgggatctgataacgggcctgccttcgcctcccaggtaagtcagtcagtggccgatttactggggatcgattggaagttacattgtgcttatagaccccagagttcaggacaggtagaaagaatgaatagaacaattaaggagactttgaccaaattaacgcttgcatctggcactagagactgggtactcctactccccttagccctctaccgagcccggaatactccgggcccccacggactgactccgtatgaaattctgtatggggcacccccgccccttgtcaattttcatgatcctgaaatgtcaaagttaactaatagtccctctctccaagctcacttacaggccctccaagcagtacaacaagaggtctggaagccgctggccgctgcttatcaggaccagctagatcagccagtgataccacaccccttccgtgtcggtgacgccgtgtgggtacgccggcaccagactaagaacttagaacctcgctggaaaggaccctacaccgtcctgctgacaacccccaccgctctcaaagtagacggcatctctgcgtggatacacgccgctcacgtaaaggcggcgacaactcctccggccggaacagcatggaaagtccagcgttctcaaaaccccttaaagataagattaacccgtggggccccctgataattatggggatcttggtgagggcaggagcctcagtacaacgtgacagccctcaccaggtctttaatgtcacttggaaaattaccaacctaatgacaggacaaacagctaatgctacctccctcctggggacgatgacagacactttccctaaactatattttgacttgtgtgatttagttggagacaactgggatgacccggaacccgatattggagatggttgccgctctcccgggggaagaaaaaggacaagactatatgatttctatgtttgccccggtcatactgtattaacagggtgtggagggccgagagagggctactgtggcaaatggggatgtgagaccactggacaggcatactggaagccatcatcatcatgggacctaatttcccttaagcgaggaaacactcctaagggtcagggcccctgttttgattcctcagtgggctccggtagcatccagggtgccacaccggggggtcgatgcaaccccctagtcctagaattcactgacgcgggtaaaagggccagctgggatgcccccaaaacatggggactaagactgtatcgatccactggggccgacccggtgaccctgttctctctgacccgccaggtcctcaatgtagggccccgcgtccccattgggcctaatcccgtgatcactgaacagctacccccctcccaacccgtgcagatcatgctccccaggcctcctcgtcctcctccttcaggcgcggcctctatggtgcctggggctcccccgccttctcaacaacctgggacgggagacaggctgctaaacctggtagaaggagcctaccaagccctcaacctcaccagtcccgacaaaacccaagagtgctggctgtgtctagtatcgggacccccctactacgaaggggtggccgtcctaggtacttactccaaccatacctctgccccggctaactgctccgtgacctcccaacacaagctgaccctgtccgaagtgaccgggcagggactctgcataggagcagttcccaaaacccatcaggccctgtgtaataccacccagaagacgagcgacgggtcctactatttggcctctcccgccgggaccatttgggcttgcagcaccgggctcactccctgtctatctactactgtgcttaacttaaccactgattactgtgtcctggttgaactctggccaaaggtaacctaccactcccctaattatgtttatggccagtttgaaaagaaaactaaatataaaagagagccggtgtcattaactctggccctgctgttgggaggacttactatgggcggcatagctgcaggagttggaacagggactacagccctagtggccaccaaacaattcgagcagctccaggcagccatacatacagaccttggggccttagaaaaatcagtcagtgccctagaaaagtctctgacctcgttgtctgaggtggtcctacagaaccggaggggattagatctactgttcctaaaagaaggaggattatgtgctgccctaaaagaagaatgctgtttttacgcggaccacactggcgtagtaagagatagcatggcaaagctaagagaaaggttaaaccagagacaaaaattgttcgaatcacgacaagggtggtttgagggactgtttaacaggtccccatggttcacgaccctgatatccaccattatgggccctctgatagtacttttattaatcctactcttcggaccctgtattctcaaccgcttggtccagtttgtaaaagacagaatttcggtagtgcaggccctggttctgacccaacagtatcaccaactcaaatcaatagatccagaagaagtggaatcacgtgaataaaagatttattcagtttccagaaagaggggggaatgaaagaccccaccataaggcttagcacgctagctacagtaacgccattttgcaagcatggaaaagtaccagagctgagttctcaaaagttacaaggaagtttaattaaagaataaggctgaataacactgggacaggggccaaacaggatatctgtagtcaggcacctgggccccggctcagggccaagaacagatggtcctcagataaagcgaaactaacaacagtttctggaaagtcccacctcagtttcaagttccccaaaagaccgggaaataccccaagccttatttaaactaaccaatcagctcgcttctcgcttctgtacccgcgctttttgctccccagtcctagccctataaaaaaggggtaagaactccacactcggcgcgccagtcatccgatagactgagtcgcccgggtacccgtgttcccaataaagcctt

SEQ ID NO: 163 - DNA, 8185 bp, Xenotropic murine leukemia virus
gcgccagtcatccgatagactgagtcgcccgggtacccgtgttcccaataaagccttttgctgtttgcatccgaagcgtggcctcgctgttccttgggagggtctcctcagagtgattgactacccagctcgggggtctttcatttgggggctcgtccgggattcggagacccccgcccagggaccaccgacccaccgtcgggaggtaagccggccggcgatcgttttgtctttgtctctgtctttgtgcgtgtgtgtgtgtgccggcatctaatcctcgcgcctgcgtctgaatctgtactagttagctaactagatctgtatctggcggttccgcggaagaactgacgagttcgtattcccggccgcagcccagggagacgtcccagcggcctcgggggcccgttttgtggcccattctgtatcagttaacctacccgagtcggactctttggagtggctttgttgggggacgagagacagagacacttcccgcccccgtctgaatttttgctttcggttttacgccgaaaccgcgccgcgcgtctgatttgttttgttgttcttctgttcttcgttagttttcttctgtctttaagtgttctcgagatcatgggacagaccgtaactacccctctgagtctaaccttgcagcactggggagatgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccctctctataaagtccaaacctcctaagccccaggttctccctgatagcggcggacctctcattgaccttctcacagaggatcccccgccgtacggagtacaaccttcctcctctgccagggagaacaatgaagaagaggcggccaccacctccgaggtttcccccccttctcccatggtgtctcgactgcggggaaggagagaccctcccgcagcggactccaccacctcccaggcattcccactccgcatggggggagatggccagcttcagtactggccgttttcctcctctgatttatataattggaaaaataataacccttccttttctgaagatccaggtaaattgacggccttgattgagtccgtcctcatcacccaccagcccacctgggacgactgtcagcagttgttggggaccctgctgaccggagaagaaaagcagcgggtgctcctagaggctggaaaggcagtccggggcaatgatggacgccccactcagttgcctaatgaagtcaatgctgcttttccccttgagcgccccgattgggattacaccactacagaaggtaggaaccacctagtcctctaccgccagttgctcttagcgggtctccaaaacgcgggcaggagccccaccaatttggccaaggtaaaagggataacccagggacctaatgagtctccctcagcctttttagagagactcaaggaggcctatcgcaggtacactccttatgaccctgaggacccagggcaagaaaccaatgtgtccatgtcattcatctggcagtctgccccggatatcgggcgaaagttagagcggttagaagatttaaagagcaagaccttaggagacttagtgagggaagctgaaaagatctttaataagcgagaaaccccggaagaaagagaggaacgtatcaggagagaaatagaggaaaaagaagaacgccgtagggcagaggatgagcagagagagagagaaagggaccgcagaagacatagagagatgagcaagctcttggccactgtagttattggtcagagacaggatagacaggggggagagcggaggaggccccaacttgataaggaccaatgcgcctactgcaaagaaaagggacactgggctaaggactgcccaaagaagccacgagggccccgaggaccgaggccccagacctccctcctgaccttaggtgactagggaggtcagggtcaggagcccccccctgaacccaggataaccctcaaagtcggggggcaacccgtcaccttcctggtagatactggggcccaacactccgtgctgacccaaaatcctggacccctaagtgacaagtctgcctgggtccaaggggctactggaggaaagcggtatcgctggaccacggatcgcaaagtacatctggctaccggtaaggtcacccactctttcctccatgtaccagactgcccctatcctctgctaggaagagacttgctgactaaactaaaagcccaaatccacttcgagggatcaggagctcaggttgtgggaccgatgggacagcccctgcaagtgctgaccctaaacatagaaaataagtatcggctacatgagacctcaaaagagccagatgttcctctagggtccacatggctttctgattttccccaggcctgggcggaaaccgggggcatgggactggcagttcgccaagctcctctgatcatacctctgaaggcaacctctacccccgtgtccataaaacaataccccatgtcacaagaagccagactggggatcaagccccacatacagaggctgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaactgacctggaccagactcccacagggtttcaaaaacagtcccaccctgtttgatgaggcactgcacagagacctagcagatttccggatccagcacccagacttgatcctgctacagtacgtggatgacttactgctggccgccacttctgagcaagactgccaacgaggtactcgggccctattacaaaccctagggaacctcgggtatcgggcctcggccaagaaagcccaaatttgccagaaacaggtcaagtatctggggtatctcctaaaagagggacagagatggctgactgaggccagaaaagagactgtgatggggcagcccactccgaagacccctcgacaactaagggagttcctagggacggcaggcttctgtcgcctctggatccctgggtttgcagaaatggcagcccccttgtaccctcttaccaaaacggggactctgtttaattggggcccagaccagcaaaaggcctatcaagaaatcaaacaggctcttctaactgcccccgccctgggattgccagatttgactaagccctttgaactctttgtcgacgagaagcagggctacgccaaaggcgtcctaacgcaaaaactgggaccttggcgtcggcctgtggcctacctgtccaaaaagctagacccagtggcagctgggtggcccccttgcctacggatggtagcagccattgccgttctgacaaaggatgcaggcaagctaactatgggacagccgctagtcattctggccccccatgcggtagaagcactggtcaaacaaccccctgaccgttggctatccaatgcccgcatgacccactatcaggcaatgctcctggatacagaccgggttcagttcggaccggtggtggccctcaacccggccaccctgctccccctaccggaaaaggaagccccccatgactgcctcgagatcttggctgagacgcacggaaccagaccggacctcacggaccagcccatcccagacgctgattacacttggtacacagatggaagcagcttcctacaagaaggacaacggagagctggagcagcggtgactactgagaccgaggtaatctgggcgagggctctgccggctggaacatccgcccaacgagccgaactgatagcactcacccaagccttaaagatggcagaaggtaagaagctaaatgtttacactgatagccgctatgccttcgccacggcccatgtccatggagaaatatataggaggcgagggttgctgacctcagaaggcagagaaattaaaaacaagaacgagatcttggccttgctaaaagctctctttctgcccaaacgacttagtataattcactgtccaggacatcaaaaaggaaacagtgctgaggccagaggcaaccgtatggcagatcaagcagcccgagaggcagccatgaaggcagttctagaaacctctacactcctcatagaggactcaaccccgtatacgcctccccatttccattacaccgaaacagatctcaaaagactacgggaactgggagccacctacaatcagacaaaaggatattgggtcctacaaggcaaacctgtgatgcccgatcagtccgtgtttgaactgttagactccctacacagactcacccatccgagccctcaaaagatgaaggcactcctcgacagagaagaaagcccctactacatgttaaaccgggacagaactatccagtatgtgactgagacctgcaccgcctgtgcccaagtaaatgccagcaaagccaaaattggggcaggggtgcgagtacgcggacatcggccaggcacccattgggaagttgatttcacggaagtaaagccaggactgtatgggtacaagtacctcctagtgtttgtagacaccttctctggctgggtagaggcattcccgaccaagcgggaaactgccaaggttgtgaccaaaaagctgttagaagacatttttccgagatttggaatgccgcaggtattgggatctgataacgggcctgccttcgcctcccaggtaagtcagtcagtggccgatttactggggatcgattggaagttacattgtgcttatagaccccagagttcaggacaggtagaaagaatgaatagaacaattaaggagactttgaccaaattaacgcttgcatctggcactagagactgggtactcctactccccttagccctctaccgagcccggaatactccgggcccccacggactgactccgtatgaaattctgtatggggcacccccgccccttgtcaattttcatgatcctgaaatgtcaaagttaactaatagtccctctctccaagctcacttacaggccctccaagcagtacaacaagaggtctggaagccgctggccgctgcttatcaggaccagctagatcagccagtgataccacaccccttccgtgtcggtgacgccgtgtgggtacgccggcaccagactaagaacttagaacctcgctggaaaggaccctacaccgtcctgctgacaacccccaccgctctcaaagtagacggcatctctgcgtggatacacgccgctcacgtaaaggcggcgacaactcctccggccggaacagcatggaaagtccagcgttctcaaaaccccttaaagataagattaacccgtggggccccctgataattatggggatcttggtgagggcaggagcctcagtacaacgtgacagccctcaccaggtctttaatgtcacttggaaaattaccaacctaatgacaggacaaacagctaatgctacctccctcctggggacgatgacagacactttccctaaactatattttgacttgtgtgatttagttggagacaactgggatgacccggaacccgatattggagatggttgccgctctcccgggggaagaaaaaggacaagactatatgatttctatgtttgccccggtcatactgtattaacagggtgtggagggccgagagagggctactgtggcaaatggggatgtgagaccactggacaggcatactggaagccatcatcatcatgggacctaatttcccttaagcgaggaaacactcctaagggtcagggcccctgttttgattcctcagtgggctccggtagcatccagggtgccacaccggggggtcgatgcaaccccctagtcctagaattcactgacgcgggtaaaagggccagctgggatgcccccaaaacatggggactaagactgtatcgatccactggggccgacccggtgaccctgttctctctgacccgccaggtcctcaatgtagggccccgcgtccccattgggcctaatcccgtgatcactgaacagctacccccctcccaacccgtgcagatcatgctccccaggcctcctcgtcctcctccttcaggcgcggcctctatggtgcctggggctcccccgccttctcaacaacctgggacgggagacaggctgctaaacctggtagaaggagcctaccaagccctcaacctcaccagtcccgacaaaacccaagagtgctggctgtgtctagtatcgggacccccctactacgaaggggtggccgtcctaggtacttactccaaccatacctctgccccggctaactgctccgtgacctcccaacacaagctgaccctgtccgaagtgaccgggcagggactctgcataggagcagttcccaaaacccatcaggccctgtgtaataccacccagaagacgagcgacgggtcctactatttggcctctcccgccgggaccatttgggcttgcagcaccgggctcactccctgtctatctactactgtgcttaacttaaccactgattactgtgtcctggttgaactctggccaaaggtaacctaccactcccctaattatgtttatggccagtttggaaagaaaactaaatataaaagagagccggtgtcattaactctggccctgctgttgggaggacttactatgggcggcatagctgcaggagttggaacagggactacagccctagtggccaccaaacaattcgagcagctccaggcagccatacatacagaccttggggccttagaaaaatcagtcagtgccctagaaaagtctctgacctcgttgtctgaggtggtcctacagaaccggaggggattagatctactgttcctaaaagaaggaggattatgtgctgccctaaaaaaagaatgctgtttttacgcggaccacactggcgtagtaagagatagcatggcaaagctaagagaaaggttaaaccagagacaaaaattgttcgaatcaggacaagggtggtttgagggactgtttaacaggtccccatggttcacgaccctgatatccaccattatgggccctctgatagtacttttattaatcctactcttcggaccctgtattctcaaccgcttggtccagtttgtaaaagacagaatttcggtagtgcaggccctggttctgacccaacagtatcaccaactcaaatcaatagatccagaagaagtggaatcacgtgaataaaagattttattcagtttccagaaagaggggggaatgaaagaccccaccataaggcttagcacgctagctacagtaacgccattttgcaaggcatggaaaagtaccagagctgagttctcaaaagttacaaggaagtttaattaaagaataaggctgaataacactgggacaggggccaaacaggatatctgtagtcaggcacctgggccccggctcagggccaagaacagatggtcctcagataaagcgaaactaacaacagtttctggaaagtcccacctcagtttcaagttccccaaaagaccgggaaataccccaagccttatttaaactaaccaatcagctcgcttctcgcttctgtacccgcgctttttgctccccagtcctagccctataaaaaaggggtaagaactccacactcggcgcgccagtcatccgatagactgagtcgcccgggtacccgtgttcccaataaagccttttgctgtttgcaa

SEQ ID NO: 164 - DNA, 8185 bp, Xenotropic murine leukemia virus
gcgccagtcatccgatagactgagtcgcccgggtacccgtgttcccaataaagccttttgctgtttgcatccgaagcgtggcctcgctgttccttgggagggtctcctcagagtgattgactacccagctcgggggtctttcatttgggggctcgtccgggattcggagacccccgcccagggaccaccgacccaccgtcgggaggtaagccggccggcgatcgttttgtctttgtctctgtctttgtgcgtgtgtgtgtgtgccggcatctaatcctcgcgcctgcgtctgaatctgtactagttagctaactagatctgtatctggcggttccgcggaagaactgacgagttcgtattcccggccgcagccctgggagacgtcccagcggcctcgggggcccgttttgtggcccattctgtatcagttaacctacccgagtcggactttttggagtggctttgttgggggacgagagacagagacacttcccgcccccgtctgaatttttgctttcggttttacgccgaaaccgcgccgcgcgtctgatttgttttgttgttcttctgttcttcgttagttttcttctgtctttaagtgttctcgagatcatgggacagaccgtaactacccctctgagtctaaccttgcagcactggggagatgtccagcgcattgcatccaaccagtctgtggatgtcaagaagaggcgctgggttaccttctgttccgccgaatggccaactttcaatgtaggatggcctcaggatggtacttttaatttaggtattatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccccctctataaagtccaaacctcctaagccccaggttctccctgatagcggcggacctctcattgaccttctcacagaggatcccccgccgtacggagcacaaccttcctcctctgccagggagaacaatgaagaagaggcggccaccacctccgaggtttcccccccttctcccatggtgtctcgactgcggggaaggagagaccctcccgcagcggactccaccacctcccaggcattcccactccgcatggggggagatggccagcttcagtactggccgttttcctcctctgatttatataattggaaaaataataacccttccttttctgaagatccaggtaaattgacggccttgattgagtccgtcctcatcacccaccagcccacctgggacgactgtcagcagttgttggggaccctgctgaccggagaagaaaagcagcgggtgctcctagaggctagaaaggcagtccggggcaatgatggacgccccactcagttgcctaatgaagtcaatgctgcttttccccttgagcgccccgattggggttacaccactacagaaggtaggaaccacctagtcctctaccgccagttgctcttagcgggtctccaaaacgcgggcaggagccccaccaatttggccaaggtaaaagggataacccagggacctaatgagtctccctcagcctttttagagagactcaaggaggcctatcgcaggtacactccttatgaccctgaggacccagggcaagaaaccaatgtgtccatgtcattcatctggcagtctgccccggatatcgggcgaaagttagagcggttagaagatttaaagagcaagaccttaggagacttagtgagggaagctgaaaagatctttaataagcgagaaaccccggaagaaagagaggaacgtatcaggagagaaatagaggaaaaagaagaacgccgtagggcagaggatgagcagagagagagagaaagggaccgcagaagacatagagagatgagcaagctcttggccactgtagttattggtcagagacaggatagacaggggggagagcggaggaggccccaacttgataaggaccaatgcgcctactgcaaagaaaagggacactgggctaaggactgcccaaagaagccacgagggccccgaggaccgaggccccagacctccctcctgaccttaggtgactagggaggtcagggtcaggagcccccccctgaacccaggataaccctcaaagtcggggggcaacccgtcaccttcctggtagatactggggcccaacactccgtgctgacccaaaatcctggacccctaagtgacaagtctgcctgggtccaaggggctactggaggaaagcggtatcgctggaccacggatcgcaaagtacatctggctaccggtaaggtcacccactctttcctccatgtaccagactgcccctatcctctgctaggaagagacttgctgactaaactaaaagcccaaatccacttcgagggatcaggagctcaggttgtgggaccgatgggacagcccctgcaagtgctgaccctaaacatagaagatgagtatcggctacatgagacgtcaaaagagccagatgttcctctagggtccacatggctttctgattttccccaggcctgggcggaaaccgggggcatgggactggcagttcgccaagctcctctgatcatacctctgaaggcaacctctacccccgtgtccataaaacaataccccatgtcacaagaagccagactggggatcaagccccacatacagaggctgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaactgacctggaccagactcccacagggtttcaaaaacagtcccaccctgtttgatgaggcactgcacagagacctagcagatttccggatccagcacccagacttgatcctgctacagtacgtggatgacttactgctggccgccacttctgagcaagactgccaacgaggtactcgggccctattacaaaccctagggaacctcgggtatcgggcctcggccaagaaagcccaaatttgccagaaacaggtcaagtatctggggtatctcctaaaagagggacagagatggctgactgaggccagaaaagagactgtgatggggcagcccactccgaagacccctcgacaactaagggagttcctagggacggcaggcttctgtcgcctctggatccctgggtttgcagaaatggcagcccccttgtaccctcttaccaaaacggggactctgtttaattggggcccagaccagcaaaaggcctatcaagaaatcaaacaggctcttctaactgcccccgccctgggattgccagatttgactaagccctttgaactctttgtcgacgagaagcagggctacgccaaaggcgtcctaacgcaaaaactgggaccttggcgtcggcctgtggcctacctgtccaaaaagctagacccagtggcagccgggtggcccccttgcctacggatggtagcagccattgccgttctgacaaaggatgcaggcaagctaactatgggacagccgctagtcattctggccccccatgcggtagaagcactggtcaaacaaccccctgaccgttggctatccaatgcccgcatgacccactatcaggcaatgctcctggatacagaccgggttcagttcggaccggtggtggccctcaacccggccaccctgctccccctaccggaaaaggaagccccccatgactgcctcgagatcttggctgagacgcacggaaccagaccggacctcacggaccagcccatcccagacgctgattacacttggtacacagatggaggcagcttcctacaagaaggacaacggagagctggagcagcggtgactactgagaccgaggtaatctggggaggggttctgccggctggaacatccgcccaacgagccgaactgatagcactcacccaagccttaaagatggcagaaggtaagaagctaaatgtttacactgatagccgctatgccttcgccacggcccatgtccatggagaaatatataggaggcgagggttgctgacctcagaaggcagagaaattaaaaacaagaacgagatcttggccttgctaaaagctctctttctgcccaaacgacttagtataattcactgtccaggacatcaaaaaggaaacagtgctgaggccagaggcaaccgtatggcagatcaagcagcccgagaggcagccatgaaggcagttctagaaacctctacactcctcatagaggactcaaccccgtatacgcctccccatttccattacaccgaaacagatctcaaaagactacgggaactgggagccacctacaatcagacaaaaggatattgggtcctacaaggcaaacctgtgatgcccgatcagtccgtgtttgaactgttagactccctacacagactcacccatctgagccctcaaaagatgaaggcactcctcgacagagaagaaagcccctactacatgttaaaccgggacagaactatccagtatgtgactgagacctgcaccgcctgtgcccaagtaaatgccagcaaagccaaaattggggcaggggtgcgagtacgcggacatcggccaggcacccattgggaagttgatttcacggaagtaaagccaggactgtatgggtacaagtacctcctagtgtttgtagacaccttctctggctgggtagaggcattcccgaccaagcgggaaactgccaaggtcgtgtccaaaaagctgttagaagacatttttccgagatttggaatgccgcaggtattgggatctgataacgggcctgccttcgcctcccaggtaagtcagtcagtggccgatttactggggatcgattggaagttacattgtgcttatagaccccagagttcaggacaggtagaaagaatgaatagaacaattaaggagactttgaccaaattaacgcttgcatctggcactagagactgggtactcctactccccttagccctctaccgagcccggaatactccgggcccccacggactgactccgtatgaaattctgtatggggcacccccgccccttgtcaattttcatgatcctgaaatgtcaaagttaactaatagtccctctctccaagctcacttacaggccctccaagcagtacaacaagaggtctggaagccgctggccgctgcttatcaggaccagctagatcagccagtgataccacaccccttccgtgtcggtgacgccgtgtgggtacgccggcaccagactaagaacttagaacctcgctggaaaggaccctacaccgtcctgctgacaacccccaccgctctcaaagtagacggcatctctgcgtggatacacgccgctcacgtaaaggcggcgacaactcctccggccggaacagcatggaaagtccagcgttctcaaaaccccttaaagataagattaacccgtggggccccctgataattatggggatcttggtgagggcaggagcctcagtacaacgtgacagccctcaccaggtctttaatgtcacttggaaaattaccaacctaatgacaggacaaacagctaatgctacctccctcctggggacgatgacagacactttccctaaactatattttgacttgtgtgatttagttggagacaactgggatgacccggaacccgatattggagatggttgccgctctcccgggggaagaaaaaggacaagactatatgatttctatgtttgccccggtcatactgtattaacagggtgtggagggccgagagagggctactgtggcaaatggggatgtgagaccactggacaggcatactggaagccatcatcatcatgggacctaatttcccttaagcgaggaaacactcctaagggtcagggcccctgttttgattcctcagtgggctccggtagcatccagggtgccacaccggggggtcgatgcaaccccctagtcctagaattcactgacgcgggtaaaagggccagctgggatgcccccaaaacatggggactaagactgtatcgatccactggggccgacccggtgaccctgttctctctgacccgccaggtcctcaatgtagggccccgcgtccccattgggcctaatcccgtgatcactgaacagctacccccctcccaacccgtgcagatcatgctccccaggcctcctcgtcctcctccttcaggcgcggcctctatggtgcctggggctcccccgccttctcaacaacctgggacgggagacaggctgctaaacctggtagaaggagcctaccaagcactcaacctcaccagtcccgacaaaacccaagagtgctggctgtgtctagtatcgggacccccctactacgaaggggtggccgtcctaggtacttactccaaccatacctctgccccggctaactgctccgtgacctcccaacacaagctgaccctgtccgaagtgaccgggcagggactctgcataggagcagttcccaaaacccatcaggccctgtgtaataccacccagaagacgagcgacgggtcctactatttggcctctcccgccgggaccatttgggcttgcagcaccgggctcactccctgtctatctactactgtgcttaacttaaccactgattactgtgtcctggttgaactctggccaaaggtaacctaccactcccctaattatgtttatggccagtttgaaaagaaaactaaatataaaagagagccggtgtcattaactctggccctgctgttgggaggacttactatgggcggcatagctgcaggagttggaacagggactacagccctagtggccaccaaacaattcgagcagctccaggcagccatacatacagaccttggggccttagaaaaatcagtcagtgccctagaaaagtctctgacctcgttgtctgaggtggtcctacagaaccggaggggattagatctactgttcctaaaagaaggaggattatgtgctgccctaaaagaagaatgctgtttttacgcggaccacactggcgtagtaagagatagcatggcaaagctaagagaaaggttaaaccagagacaaaaattgttcgaatcaggacaagggtggtttgagggactgtttaacaggtccccatggttcacgaccctgatatccaccattatgggccctctgatagtacttttattaatcctactcttcggaccctgtattctcaaccgcttggtccagtttgtaaaagacagaatttcggtagtgcaggccctggttctgacccaacagtatcaccaactcaaatcaatagatccagaagaagtggaatcacgtgaataaaagattttattcagtttccagaaagaggggggaatgaaagaccccaccataaggcttagcacgctagctacagtaacgccattttgcaaggcatggaaaagtaccagagctgagttctcaaaagttacaaggaagtttaattaaagaataaggctgaataacactgggacaggggccaaacaggatatctgtagtcaggcacctgggccccggctcagggccaagaacagatggtcctcagataaagcgaaactaacaacagtttctggaaagtcccacctcagtttcaagttccccaaaagaccgggaaataccccaagccttatttaaactaaccaatcagctcgcttctcgcttctgtacccgcgctttttgctccccagtcctagccctataaaaaaggggtaagaactccacactcggcgcgccagtcatccgatagactgagtcgcccgggtacccgtgttcccaataaagccttttgctgtttgcaa

SEQ ID NO: 165 - DNA, 490 bp, Xenotropic murine leukemia virus; identical to SEQ ID NO: 44

SEQ ID NO: 166 - DNA, 490 bp, Xenotropic murine leukemia virus; identical to SEQ ID NO: 44

SEQ ID NO: 167 - protein, 162 aa, Xenotropic murine leukemia virus
PWGPLIIMGILVRAGASVQRDSPHQVFNVTWKITNLMTGQTANATSLLGTMTDTFPKLYFDLCDLVGDNWDDPEPDIGDGCRSPGGRKRTRLYDFYVCPGHTVLTGCGGPREGYCGKWGCETTGQAYKPSSSWDLISLKRGNTPKGQGPCFDSSVGSGSIQG

SEQ ID NO: 168 - protein, 162 aa, Xenotropic murine leukemia virus; identical to SEQ ID NO: 167

SEQ ID NO: 169 - DNA, 321 bp, Xenotropic murine leukemia virus
caattcgagcagctccaggcagccatacatacagaccttggggccttagaaaaatcagtcagtgccctagaaaagtctctgacctcgttgtctgaggtggtcctacagaaccggaggggattagatcactgttcctaaaagaaggaggattatgtgctgccctaaaagaagaatgctgtttttacgcggaccacactggcgtagtaagagatagcatggcaaagctaagagaaaggttaaaccagagacaaaaattgttcgaatcaggacaagggtggtttgagggactgtttaacaggtccccatggttcacgaccctga

SEQ ID NO: 170 - DNA, 321 bp, Xenotropic murine leukemia virus
caattcgagcagctccaggcagccatacatacagaccttggggccttagaaaaatcagtcagtgccctagaaaagtctctgacctcgttgtctgaggtggtcctacagaaccggaggggattagatcactgttcctaaaagaaggaggattatgtgctgccctaaaaaaagaatgctgtttttacgcggaccacactggcgtagtaagagatagcatggcaaagctaagagaaaggttaaaccagagacaaaaattgttcgaatcaggacaagggtggtttgagggactgtttaacaggtccccatggttcacgaccctga

SEQ ID NO: 171 - protein, 108 aa, Xenotropic murine leukemia virus
  misc_feature (108)..(108): Xaa can be any naturally occurring amino acid
QFEQLQAAIHTDLGALEKSVSALEKSLTSLSEVVLQNRRGLDLLFLKEGGLCAALKEECCFYADHTGVVRDSMAKLRERLNQRQKLFESGQGWFEGLFNRSPWFTTLX

SEQ ID NO: 172 - protein, 108 aa, Xenotropic murine leukemia virus
  misc_feature (108)..(108): Xaa can be any naturally occurring amino acid
QFEQLQAAIHTDLGALEKSVSALEKSLTSLSEVVLQNRRGLDLLFLKEGGLCAALKKECCFYADHTGVVRDSMAKLRERLNQRQKLFESGQGWFEGLFNRSPWFTTLX

SEQ ID NO: 173 - DNA, 370 bp, Xenotropic murine leukemia virus
ctacccctctgagtctaaccttgcagcactggggagatgtccagcgcattgcatccaaccagtctgtggatgtcaagaagaggcgctgggttaccttctgttccgccgaatggccaactttcaatgtggatggcctcaggatggtacttttaatttaggtattatctctcaggtcaagtctagagtgttttgtcctggtccccacggacacccggatcaggtcccatatatcgtcacctgggaggcacttgcctatgacccccctccgtgggtcaaccgtttgtctctcctaaaccccctcctttaccgacagctcccgtcctcccgcccggtccttctgcgcaacctccgtcccgatctgccctttacc

SEQ ID NO: 174 - DNA, 370 bp, Xenotropic murine leukemia virus
ctacccctctgagtctaaccttgcagcactggggagatgtccagcgcattgcatccaaccagtctgtggatgtcaagaagaggcgctgggttaccttctgttccgccgaatggccaactttcaatgtggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaccgtttgtctctcctaaaccccctcctttaccgacagctcccgtcctcccgcccggtccttctgcgcaacctccgtcccgatctgccctttacc

SEQ ID NO: 175 - protein, 123 aa, Xenotropic murine leukemia virus
TPLSLTLQHWGDVQRIASNQSVDVKKRRWVTFCSAEWPTFNVGWPQDGTFNLGIISQVKSRVFCPGPHGHPDQVPYIVTWEALAYDPPPWVKPFVSPKPPPLPTAPVLPPGPSAQPPSRSALY

SEQ ID NO: 176 - protein, 123 aa, Xenotropic murine leukemia virus
TPLSLTLQHWGDVQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALY

SEQ ID NO: 177 - DNA, 374 bp, Xenotropic murine leukemia virus
tctaccttgcagcactggggagtgtccagcgcattgcatccaaccagtctgtggatgtcaagaagaggcgctgggttaccttctgttccgccgaatggccaactttcaatgtaggatggcctcaggaggtacttttaatttaggtgttatctctcaggtcaagtctagagtgttttgtcctggtccccacggacacccggatcaggtcccatatatcgtcacctgggaggcacttgcctatgacccccctccgtgggtcaaccgtttgtctctcctaaaccccctcctttaccgacagctcccgtcctcccgcccggtccttctgcgcaacctccgtcccgatctgccctttaccctgccatttaccctctgaa

SEQ ID NO: 178 - protein, 124 aa, Xenotropic murine leukemia virus
YLAALGSVQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPAIYPL

SEQ ID NO: 179 - DNA, 59 bp, Xenotropic murine leukemia virus
gaaaagtctctgacctcgttgtctgaggtggtcctacagaaccggaggggattagtcta

SEQ ID NO: 180 - protein, 20 aa, Xenotropic murine leukemia virus
GLDLEKSLTSLSHVVLQNRR